(19) United States
(12) Patent Application Publication
(10) Pub. No.: US 2024/0299569 A1
(43) Pub. Date: Sep. 12, 2024

(54) 2-AMINO-4-CARBOXAMIDE-BENZAZEPINE IMMUNOCONJUGATES, AND USES THEREOF

(71) Applicant: BOLT BIOTHERAPEUTICS, INC., Redwood City, CA (US)

(72) Inventors: Gary BRANDT, Redwood City, CA (US); Romas KUDIRKA, Redwood City, CA (US); Brian SAFINA, Redwood City, CA (US)

(73) Assignee: BOLT BIOTHERAPEUTICS, INC., Redwood City, CA (US)

(21) Appl. No.: 18/283,361

(22) PCT Filed: Mar. 25, 2022
(86) PCT No.: PCT/US2022/021985

§ 371 Date: Sep. 21, 2023

Related U.S. Application Data
(60) Provisional application No. 63/166,710, filed on Mar. 26, 2021.

(51) Int. Cl. A61K 47/68; A61K 47/54; A61K 47/60

(52) U.S. Cl. CPC A61K 47/6803; A61K 47/545; A61K 47/60; A61K 47/6849; A61K 47/6851

(57) ABSTRACT

The invention provides immunoconjugates of Formula I comprising an antibody linked by conjugation to one or more 2-amino-4-carboxamide-benzazepine derivatives. The invention also provides 2-amino-4-carboxamide-benzazepine derivative intermediate compositions comprising a reactive functional group. Such intermediate compositions are suitable substrates for formation of the immunoconjugates through a linker or linking moiety. The invention further provides methods of treating cancer with the immunoconjugates.

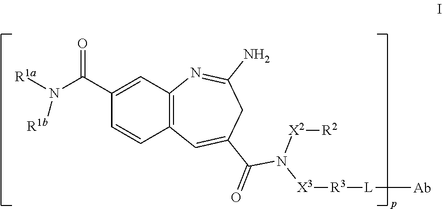

### CROSS REFERENCE TO RELATED APPLICATIONS

This non-provisional application claims the benefit of priority to U.S. Provisional Application No. 63/166,710, filed 26 Mar. 2021, which is incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 25, 2022, is named 17019_015WO1_SL.txt and is 85,369 bytes in size.

### FIELD OF THE INVENTION

The invention relates generally to an immunoconjugate comprising an antibody conjugated to one or more 2-amino-4-carboxamide-benzazepine molecules.

### BACKGROUND OF THE INVENTION

New compositions and methods for the delivery of antibodies and dendritic cell/myeloid cell adjuvants are needed in order to reach inaccessible tumors and/or to expand treatment options for cancer patients and other subjects. The invention provides such compositions and methods.

### SUMMARY OF THE INVENTION

The invention is generally directed to immunoconjugates comprising an antibody linked by conjugation to one or more 2-amino-4-carboxamide-benzazepine derivatives. The invention is further directed to 2-amino-4-carboxamide-benzazepine derivative intermediate compositions comprising a reactive functional group. Such intermediate compositions are suitable substrates for formation of immunoconjugates wherein an antibody may be covalently bound by a linker L to the 4-position of an 2-amino-4-carboxamide-benzazepine moiety having the formula:

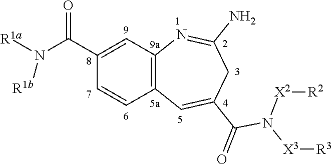

where R³ is attached to the linker L. The positions of the 3H-benzo[b]azepine structure are numbered according to IUPAC conventions. The X²⁻³ and R¹⁻³ substituents are defined herein.

The antibody binds to a target selected from the group consisting of PD-L1, HER2, TROP2, and CEA.

The invention is further directed to use of such an immunoconjugates in the treatment of an illness, in particular cancer.

An aspect of the invention is an immunoconjugate comprising an antibody covalently attached to a linker which is covalently attached to one or more 2-amino-4-carboxamide-benzazepine moieties.

Another aspect of the invention is a 2-amino-4-carboxamide-benzazepine-linker compound.

Another aspect of the invention is a method for treating cancer comprising administering a therapeutically effective amount of an immunoconjugate comprising an antibody linked by conjugation to one or more 2-amino-4-carboxamide-benzazepine moieties.

Another aspect of the invention is a use of an immunoconjugate comprising an antibody linked by conjugation to one or more 2-amino-4-carboxamide-benzazepine moieties for treating cancer.

Another aspect of the invention is a method of preparing an immunoconjugate by conjugation of one or more 2-amino-4-carboxamide-benzazepine moieties with an antibody.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively.

IgG antibodies are large molecules of about 150 kDa composed of four peptide chains. IgG antibodies contain two identical class 7 heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Bispecific” antibodies (bsAbs) are antibodies that bind two distinct epitopes to cancer (Suurs F. V. et al (2019) Pharmacology & Therapeutics 201: 103-119). Bispecific antibodies may engage immune cells to destroy tumor cells, deliver payloads to tumors, and/or block tumor signaling pathways. An antibody that targets a particular antigen includes a bispecific or multispecific antibody with at least one antigen binding region that targets the particular antigen.

In some embodiments, the targeted monoclonal antibody is a bispecific antibody with at least one antigen binding region that targets tumor cells. Such antigens include but are not limited to: mesothelin, prostate specific membrane antigen (PSMA), HER2, TROP2, CEA, EGFR, 5T4, Nectin4, CD19, CD20, CD22, CD30, CD70, B7H3, B7H4 (also known as 08E), protein tyrosine kinase 7 (PTK7), glypican-3, RG1, fucosyl-GMl, CTLA-4, and CD44 (WO 2017/196598).

An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells. Such antigens include CD47, SIRPalpha, Dectin-2, PD-1, and PD-L1.

“Antibody construct” refers to an antibody or a fusion protein comprising (i) an antigen binding domain and (ii) an Fc domain.

The term “immunoconjugate” refers to an antibody construct that is covalently bonded to an adjuvant moiety via a linker. Immunoconjugates allow targeted delivery of an active adjuvant moiety while the target antigen is bound.

“Adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

“Cysteine-mutant antibody” is an antibody in which one or more amino acid residues of an antibody are substituted with cysteine residues. A cysteine-mutant antibody may be prepared from the parent antibody by antibody engineering methods (Junutula, et al., (2008b) Nature Biotech., 26(8):925-932; Doman et al. (2009) Blood 114(13):2721-2729; U.S. Pat. Nos. 7,521,541; 7,723,485; US 2012/0121615; WO 2009/052249). Cysteine residues provide for site-specific conjugation of a adjuvant such as a TLR agonist to the antibody through the reactive cysteine thiol groups at the engineered cysteine sites but do not perturb immunoglobulin folding and assembly or alter antigen binding and effector functions. Cysteine-mutant antibodies can be conjugated to the TLR agonist-linker compound with uniform stoichiometry of the immunoconjugate (e.g., up to two TLR agonist moieties per antibody in an antibody that has a single engineered, mutant cysteine site). The TLR agonist-linker compound has a reactive electrophilic group to react specifically with the free cysteine thiol groups of the cysteine-mutant antibody.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

As used herein, the phrase “immune checkpoint inhibitor” refers to any modulator that inhibits the activity of the immune checkpoint molecule. Immune checkpoint inhibitors can include, but are not limited to, immune checkpoint molecule binding proteins, small molecule inhibitors, antibodies (including bispecific and multispecific antibodies with at least one antigen binding region that targets an immune checkpoint protein, e.g., bispecific or multispecific antibodies that do not exclusively target immune checkpoint proteins, as well as antibodies that are dual immunomodulators (simultaneous targeting two immunomodulating targets), which result in blockade of inhibitory targets, depletion of suppressive cells, and/or activation of effector cells; tumor-targeted immunomodulators (directs potent costimulation to the tumor-infiltrating immune cells by targeting a tumor antigen and costimulatory molecules such as CD40 or 4-1BB); NK-cell redirectors (redirects NK cells to malignant cells by targeting a tumor antigen and CD16A); or T-cell redirectors (redirects T cells to malignant cells by targeting a tumor antigen and CD3)), antibody-derivatives (including Fc fusions, Fab fragments, and scFvs), antibody-drug conjugates, antisense oligonucleotides, siRNA, aptamers, peptides and peptide mimetics.

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100× [(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The binding agent comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The binding agent can be any of a variety of types of binding agents known in the art that comprise Ig heavy and light chains. For instance, the binding agent can be an antibody, an antigen-binding antibody “fragment,” or a T-cell receptor.

“Biosimilar” refers to an approved antibody construct that has active properties similar to, for example, a PD-L1-targeting antibody construct previously approved such as atezolizumab (TECENTRIQ™, Genentech, Inc.), durvalumab (IMFINZI™, AstraZeneca), and avelumab (BAVENCIO™, EMD Serono, Pfizer); a HER2-targeting antibody construct previously approved such as trastuzumab (HERCEPTIN™, Genentech, Inc.), and pertuzumab (PERJETA™, Genentech, Inc.); or a CEA-targeting antibody such as labetuzumab (CEA-CIDE™, MN-14, hMN14, Immunomedics) CAS Reg. No. 219649-07-7).

“Biobetter” refers to an approved antibody construct that is an improvement of a previously approved antibody construct, such as atezolizumab, durvalumab, avelumab, trastuzumab, pertuzumab, and labetuzumab. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in a compound or material. For example, the linking moiety can serve to covalently bond an adjuvant moiety to an antibody construct in an immunoconjugate.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line [symbol] represents a point of attachment of the specified chemical moiety. If the specified chemical moiety has two wavy lines [symbol] present, it will be understood that the chemical moiety can be used bilaterally, i.e., as read from left to right or from right to left. In some embodiments, a specified moiety having two wavy lines [symbol] present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—C_{H}═CH₂), allyl (—CH₂C_{H}═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—C_{H}═C_{H}—), allyl (—CH₂C_{H}═C_{H}—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡C_{H}), propynyl (propargyl, —CH₂C≡C_{H}), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbomadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6]system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur.

Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

“PD-L1 expression” refers to a cell that has a PD-L1 receptor on the cell's surface. As used herein “PD-L1 overexpression” refers to a cell that has more PD-L1 receptors as compared to corresponding non-cancer cell.

“HER2” refers to the protein human epidermal growth factor receptor 2.

“HER2 expression” refers to a cell that has a HER2 receptor on the cell's surface. For example, a cell may have from about 20,000 to about 50,000 HER2 receptors on the cell's surface. As used herein “HER2 overexpression” refers to a cell that has more than about 50,000 HER2 receptors. For example, a cell 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed in about 25% to about 30% of breast cancers.

“TROP2 expression” refers to a cell that has a TROP2 receptor on the cell's surface. As used herein “TROP2 expression” refers to a cell that has more TROP2 receptors as compared to a corresponding normal, non-cancer cell. It is estimated that TROP2 is overexpressed in about 74% breast cancers, 72% colorectal cancers, and 64% lung cancers, and other organ types of cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety.

Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to L1X. A reference to “about X” or “around X” specifically indicates at least the values X, 0.95X, 0.96X, 0.97X, 0.98X, 0.99X, L1X, 1.02X, 1.03X, 1.04X, and 1.05X. Accordingly, “about X” and “around X” are intended to teach and provide written description support for a claim limitation of, e.g., “0.98X.”

### Antibodies

The immunoconjugate of the invention comprises an antibody. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing PD-L1, HER2, CEA, or TROP2 to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

In some embodiments, the antibodies in the immunoconjugates contain a modified Fc region, wherein the modification modulates the binding of the Fc region to one or more Fc receptors.

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FeyRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (Jefferis et al., (2009) mAbs, 1(4):332-338).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/1332E), GASDALIE (G236A/S239D/A330L/I332E), V9 (G237D/P238D/P271G/A330R), and V11 (G237D/P238D/H268D/P271G/A330R), and/or one or more mutations at the following amino acids: E233, G237, P238, H268, P271, L328 and A330. Additional Fc region modifications for modulating Fc receptor binding are described in, for example, US 2016/0145350 and U.S. Pat. Nos. 7,416,726 and 5,624,821, which are hereby incorporated by reference in their entireties.

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while defucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726 and U.S. Patent Application Publications 2007/0014795 and 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

In an exemplary embodiment, the immunoconjugates of the invention comprise an antibody construct that comprises an antigen binding domain that specifically recognizes and binds PD-L1.

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers, or infections. PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (EVIFINZI™), and avelumab (BAVENCIO™). Nevertheless, there continues to be a need for new PD-L1-binding agents, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

A method is provided of delivering a TLR agonist, 2-amino-4-carboxamide-benzazepine payload to a cell expressing PD-L1 comprising administering to the cell, or mammal comprising the cell, an immunoconjugate comprising an anti-PD-L1 antibody covalently attached to a linker which is covalently attached to one or more 2-amino-4-carboxamide-benzazepinemoieties.

Also provided is a method for enhancing or reducing or inhibiting an immune response in a mammal, and a method for treating a disease, disorder, or condition in a mammal that is responsive to PD-L1 inhibition, which methods comprise administering a PD-L1 immunoconjugate thereof, to the mammal.

The invention provides a PD-L1 antibody comprising an immunoglobulin heavy chain variable region polypeptide and an immunoglobulin light chain variable region polypeptide. The PD-L1 antibody specifically binds PD-L1. The binding specificity of the antibody allows for targeting PD-L1 expressing cells, for instance, to deliver therapeutic payloads to such cells. In some embodiments, the PD-L1 antibody binds to human PD-L1. However, antibodies that bind to any PD-L1 fragment, homolog or paralog also are encompassed.

In some embodiments, the PD-L1 antibody binds PD-L1 without substantially inhibiting or preventing PD-L1 from binding to its receptor, PD-1. However, in other embodiments, the PD-L1 antibody can completely or partially block (inhibit or prevent) binding of PD-L1 to its receptor, PD-1, such that the antibody can be used to inhibit PD-L1/PD-1 signaling (e.g., for therapeutic purposes). The antibody or antigen-binding antibody fragment can be monospecific for PD-L1, or can be bispecific or multi-specific. For instance, in bivalent or multivalent antibodies or antibody fragments, the binding domains can be different targeting different epitopes of the same antigen or targeting different antigens. Methods of constructing multivalent binding constructs are known in the art. Bispecific and multispecific antibodies are known in the art. Furthermore, a diabody, triabody, or tetrabody can be provided, which is a dimer, trimer, or tetramer of polypeptide chains each comprising a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a multimeric molecule having two, three, or four functional antigen binding sites. Also, bis-scFv fragments, which are small scFv fragments with two different variable domains can be generated to produce bispecific bis-scFv fragments capable of binding two different epitopes. Fab dimers (Fab2) and Fab trimers (Fab3) can be produced using genetic engineering methods to create multispecific constructs based on Fab fragments.

The PD-L1 antibody can be, or can be obtained from, a human antibody, a non-human antibody, a humanized antibody, or a chimeric antibody, or corresponding antibody fragments. A “chimeric” antibody is an antibody or fragment thereof typically comprising human constant regions and non-human variable regions. A “humanized” antibody is a monoclonal antibody typically comprising a human antibody scaffold but with non-human origin amino acids or sequences in at least one CDR (e.g., 1, 2, 3, 4, 5, or all six CDRs).

The PD-L1 antibody can be internalizing, as described in WO 2021/150701 and incorporated by reference herein, or the PD-L1 antibody can be non-internalizing, as described in WO 2021/150702 and incorporated by reference herein.

In an exemplary embodiment, the immunoconjugates of the invention comprise an antibody construct that comprises an antigen binding domain that specifically recognizes and binds HER2.

A number of anti-HER2 monoclonal antibodies are approved and under clinical development (Costa, R L B et al (2020) Breast Cancer 6(10):1-11.

In certain embodiments, immunoconjugates of the invention comprise an anti-HER2 antibody such as those prepared by the methods of Example 201. In one embodiment of the invention, an anti-HER2 antibody of an immunoconjugate of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J. Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an immunoconjugate of the invention comprises a humanized anti-HER2 antibody, e.g., humanized 2C4, as described in U.S. Pat. No. 7,862,817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

Margetuximab (MGAH22, MARGENZA™, MacroGenics, Inc.), CAS Reg. No. 1350624-75-7, is an FDA-approved anti-HER2 monoclonal antibody. The Fc region of margetuximab is optimized for increased binding to the activating Fe gamma Rs but decreased binding to the inhibitory Fc.gamma.Rs on immune effector cells (Nordstrom, J L, et al (2011) Breast CancerRes. 13(6):R123; Rugo, H S, et al (2021) JAMA Oncol.; 7(4):573-584; Markham, A. (2021) Drugs 81:599-604). Margetuximab is approved by the FDA for treatment of patients with relapsed or refractory advanced breast cancer whose tumors express HER2 at the 2+ level by immunohistochemistry and lack evidence of HER2 gene amplification by FISH.

HT-19 is another anti-HER2 monoclonal antibody that binds to an epitope in human HER2 distinct from the epitope of trastuzumab or pertuzumab. HT-19 was shown to inhibit HER2 signaling comparable to trastuzumab and enhance HER2 degradation in combination with trastuzumab and pertuzumab. XMT-1522 is an antibody-drug conjugate comprising the HT-19 antibody (Bergstrom D. A. et al., (2015) Cancer Res.; 75:LB-231).

In an exemplary embodiment, the immunoconjugates of the invention comprise an antibody construct that comprises an antigen binding domain that specifically recognizes and binds CEA. Carcinoembryonic antigen-related cell adhesion molecule 5 (CEACAM5) also known as CD66e (Cluster of Differentiation 66e), is a member of the carcinoembryonic antigen (CEA) gene family.

Elevated expression of carcinoembryonic antigen (CEA, CD66e, CEACAM5) has been implicated in various biological aspects of neoplasia, especially tumor cell adhesion, metastasis, the blocking of cellular immune mechanisms, and having anti-apoptosis functions. CEA is a cell-surface antigen and also is used as a blood marker for many carcinomas. Labetuzumab (CEA-CIDE™, Immunomedics, CAS Reg. No. 219649-07-7), also known as MN-14 and hMN14, is a humanized IgG1 monoclonal antibody and has been studied for the treatment of colorectal cancer (Blumenthal, R. et al (2005) Cancer Immunology Immunotherapy 54(4):315-327). Labetuzumab conjugated to a camptothecin analog (labetuzumab govitecan, IMMU-130) targets CEA and is being studied in patients with relapsed or refractory metastatic colorectal cancer (Sharkey, R. et al (2018), Molecular Cancer Therapeutics 17(1):196-203; Dotan, E. et al (2017), Journal of Clinical Oncology 35(9):3338-3346). Also, labetuzumab conjugated to¹³¹I has been evaluated in clinical trials for the treatment of colon cancer and other solid malignancies (Sharkey, R. et al (1995), Cancer Research (Suppl.) 55(23):5935s-5945s; Liersch, T. et al (2005), Journal of Clinical Oncology 23(27):6763-6770; Sahlmann, C.-O. et al (2017), Cancer 123(4):638-649).

In an embodiment of the invention, the CEA-targeting antibody construct or antigen binding domain comprises the Variable light chain (VL kappa) of hMN-14/labetuzumab SEQ ID NO. 1 as disclosed in U.S. Pat. No. 6,676,924, which is incorporated by reference herein for this purpose.

SEQ ID NO. 1
DIQLTQSPSSLSASVGDRVTITCKASQDVGTSVAWYQQKPGKAPKLLIY
WTSTRHTGVPSRFSGSGSGTDFTFTISSLQPEDIATYYCQQYSLYRSFG
QGTKVEIK

In an embodiment of the invention, the CEA-targeting antibody construct or antigen binding domain comprises the light chain CDR (complementarity determining region) or light chain framework (LFR) sequences of hMN-14/labetuzumab SEQ ID NO. 2-8 (U.S. Pat. No. 6,676,924) (full length sequence disclosed as SEQ ID NO: 1).

| | | Resi- | | SEQ |
|---|---|---|---|---|
| Region | Sequence Fragment | dues | Length | ID NO. |
| LFR1 | DIQLTQSPSSLSASVGD | 1-23 | 23 | 2 |
| | RVTITC | | | |
| CDR-L1 | KASQDVGTSVA | 24-34 | 11 | 3 |
| LFR2 | WYQQKPGKAPKLLIY | 35-49 | 15 | 4 |
| CDR-L2 | WTSTRHT | 50-56 | 7 | 5 |
| LFR3 | GVPSRFSGSGSGTDFTF | 57-88 | 32 | 6 |
| | TISSLQPEDIATYYC | | | |
| CDR-L3 | QQYSLYRS | 89-96 | 8 | 7 |
| LFR4 | FGQGTKVEIK | 97-106 | 10 | 8 |

In an embodiment of the invention, the CEA-targeting antibody construct or antigen binding domain comprises the Variable heavy chain (VH) of hMN-14/labetuzumab SEQ ID NO. 9 as disclosed in U.S. Pat. No. 6,676,924, which is incorporated by reference herein for this purpose.

SEQ ID NO. 9
EVQLVESGGGVVQPGRSLRLSCSSSGFDFTTYWMSWVRQAPGKGLEWVA
EIHPDSSTINYAPSLKDRFTISRDNSKNTLFLQMDSLRPEDTGVYFCAS
LYFGFPWFAYWGQGTPVTVSS

In an embodiment of the invention, the CEA-targeting antibody construct or antigen binding domain comprises the heavy chain CDR (complementarity determining region) or heavy chain framework (HFR) sequences of hMN-14/labetuzumab SEQ ID NO. 10-16 (U.S. Pat. No. 6,676,924) (full length sequence disclosed as SEQ ID NO: 9).

| | | Resi- | | SEQ |
|---|---|---|---|---|
| Region | Sequence Fragment | dues | Length | ID NO. |
| HFR1 | EVQLVESGGGVVQPGRS | 1-30 | 30 | 10 |
| | LRLSCSSSGFDFT | | | |
| CDR-H1 | TYWMS | 31-35 | 5 | 11 |
| HFR2 | WVRQAPGKGLEWVA | 36-49 | 14 | 12 |
| CDR-H2 | EIHPDSSTINYAPSLKD | 50-66 | 17 | 13 |
| HFR3 | RFTISRDNSKNTLFLQM | 67-98 | 32 | 14 |
| | DSLRPEDTGVYFCAS | | | |
| CDR-H3 | LYFGFPWFAY | 99-108 | 10 | 15 |
| HFR4 | WGQGTPVTVSS | 109-119 | 11 | 16 |

In an embodiment of the invention, the CEA-targeting antibody construct or antigen binding domain comprises the Variable light chain (VL kappa) of hPR1A3 SEQ ID NO. 17 as disclosed in U.S. Pat. No. 8,642,742, which is incorporated by reference herein for this purpose.

SEQ ID NO. 17
DIQMTQSPSSLSASVGDRVTITCKASAAVGTYVAWYQQKPGKAPKLLIY
SASYRKRGVPSRFSGSGSGTDFTLTISSLQPEDFATYYCHQYYTYPLFT
FGQGTKLEIK

In an embodiment of the invention, the CEA-targeting antibody construct or antigen binding domain comprises the light chain CDR (complementarity determining region) or light chain framework (LFR) sequences of hPR1A3 SEQ ID NO. 18-24 (U.S. Pat. No. 8,642,742) (full length sequence disclosed as SEQ ID NO: 17).

| Region | Sequence Fragment | Residues | Length | SEQ ID NO. |
|---|---|---|---|---|
| LFR1 | DIQMTQSPSSLSASVGDRVTITC | 1-23 | 23 | 18 |
| CDR-L1 | KASAAVGTYVA | 24-34 | 11 | 19 |
| LFR2 | WYQQKPGKAPKLLIY | 35-49 | 15 | 20 |
| CDR-L2 | SASYRKR | 50-56 | 7 | 21 |
| LFR3 | GVPSRFSGSGSGTDFTLTISSLQPEDFATYYC | 57-88 | 32 | 22 |
| CDR-L3 | HQYYTYPLFT | 89-98 | 10 | 23 |
| LFR4 | FGQGTKLEIK | 99-108 | 10 | 24 |

In an embodiment of the invention, the CEA-targeting antibody construct or antigen binding domain comprises the heavy chain CDR (complementarity determining region) or heavy chain framework (HFR) sequences of hPR1A3 SEQ ID NO. 25-31 (U.S. Pat. No. 8,642,742) (full length sequence disclosed as SEQ ID NO: 130).

| Region | Sequence Fragment | Residues | Length | SEQ ID NO. |
|---|---|---|---|---|
| HFR1 | QVQLVQSGAEVKKPGASVKVSCKASGYTFT | 1-30 | 30 | 25 |
| CDR-H1 | EFGMN | 31-35 | 5 | 26 |
| HFR2 | WVRQAPGQGLEWMG | 36-49 | 14 | 27 |
| CDR-H2 | WINTKTGEATYVEEFKG | 50-66 | 17 | 28 |
| HFR3 | RVTFTTDTSTSTAYMELRSLRSDDTAVYYCAR | 67-98 | 32 | 29 |
| CDR-H3 | WDFAYYVEAMDY | 99-110 | 12 | 30 |
| HFR4 | WGQGTTVTVSS | 111-121 | 11 | 31 |

In an embodiment of the invention, the CEA-targeting antibody construct or antigen binding domain comprises the Variable light chain (VL kappa) of hMFE-23 SEQ ID NO. 32 as disclosed in U.S. Pat. No. 7,232,888, which is incorporated by reference herein for this purpose.

SEQ ID NO. 32
ENVLTQSPSSMSASVGDRVNIACSASSSVSYMHWFQQKPGKSPKLWIYS
TSNLASGVPSRFSGSGSGTDYSLTISSMQPEDAATYYCQQRSSYPLTFG
GGTKLEIK

In an embodiment of the invention, the CEA-targeting antibody construct or antigen binding domain comprises the light chain CDR (complementarity determining region) or light chain framework (LFR) sequences of hMFE-23 SEQ ID NO. 33-40 (U.S. Pat. No. 7,232,888). The embodiment includes two variants of LFR1, SEQ ID NO.:33 and SEQ ID NO.:34 (full length sequences disclosed as SEQ ID NOS 32 and 172, respectively, in order of appearance).

| Region | Sequence Fragment | Residues | Length | SEQ ID NO. |
|---|---|---|---|---|
| LFR1 | ENVLTQSPSSMSASVGDRVNIAC | 1-23 | 23 | 33 |
| LFR1 | EIVLTQSPSSMSASVGDRVNIAC | 1-23 | 23 | 34 |
| CDR-L1 | SASSSVSYMH | 24-33 | 10 | 35 |
| LFR2 | WFQQKPGKSPKLWIY | 34-48 | 15 | 36 |
| CDR-L2 | STSNLAS | 49-55 | 7 | 37 |
| LFR3 | GVPSRFSGSGSGTDYSLTISSMQPEDAATYYC | 56-87 | 32 | 38 |
| CDR-L3 | QQRSSYPLT | 88-96 | 9 | 39 |
| LFR4 | FGGGTKLEIK | 97-106 | 10 | 40 |

In an embodiment of the invention, the CEA-targeting antibody construct or antigen binding domain comprises the Variable heavy chain (VH) of hMFE-23 SEQ ID NO. 41 (U.S. Pat. No. 7,232,888).

SEQ ID NO. 41
QVKLEQSGAEVVKPGASVKLSCKASGFNIKDSYMHWLRQGPGQRLEWIG
WIDPENGDTEYAPKFQGKATFTTDTSANTAYLGLSSLRPEDTAVYYCNE
GTPTGPYYFDYWGQGTLVTVSS

In an embodiment of the invention, the CEA-targeting antibody construct or antigen binding domain comprises the heavy chain CDR (complementarity determining region) or heavy chain framework (HFR) sequences of hMFE-23 SEQ ID NO. 42-49 (U.S. Pat. No. 7,232,888). The embodiment includes two variants of HFR1, SEQ ID NO.:42 and SEQ ID NO.:43 (full length sequences disclosed as SEQ ID NOS 41 and 173, respectively, in order of appearance).

| Region | Sequence Fragment | Residues | Length | SEQ ID NO. |
|---|---|---|---|---|
| HFR1 | QVKLEQSGAEVVKPGASVKLSCKASGFNIK | 1-30 | 30 | 42 |
| HFR1 | QVQLVQSGAEVVKPGASVKLSCKASGFNIK | 1-30 | 30 | 43 |
| CDR-H1 | DSYMH | 31-35 | 5 | 44 |
| HFR2 | WLRQGPGQRLEWIG | 36-49 | 14 | 45 |
| CDR-H2 | WIDPENGDTEYAPKFQG | 50-66 | 17 | 46 |
| HFR3 | KATFTTDTSANTAYLGLSSLRPEDTAVYYCNE | 67-98 | 32 | 47 |
| CDR-H3 | GTPTGPYYFDY | 99-109 | 11 | 48 |
| HFR4 | WGQGTLVTVSS | 110-120 | 11 | 49 |

In an embodiment of the invention, the CEA-targeting antibody construct or antigen binding domain comprises the Variable light chain (VL kappa) of SM3E SEQ ID NO. 50 (U.S. Pat. No. 7,232,888).

SEQ ID NO. 50
ENVLTQSPSSMSVSVGDRVTIACSASSSVPYMHWLQQKPGKSPKLLIYL
TSNLASGVPSRFSGSGSGTDYSLTISSVQPEDAATYYCQQRSSYPLTFG
GGTKLEIK

In an embodiment of the invention, the CEA-targeting antibody construct or antigen binding domain comprises the light chain CDR (complementarity determining region) or light chain framework (LFR) sequences of SM3E SEQ ID NO 51-56 and 38-39 (U.S. Pat. No. 7,232,888). The embodiment includes two variants of LFR1, SEQ ID NO.:51 and SEQ ID NO.:52 (full length sequences disclosed as SEQ ID NOS 50 and 174, respectively, in order of appearance).

| Region | Sequence Fragment | Residues | Length | SEQ ID NO. |
|---|---|---|---|---|
| LFR1 | ENVLTQSPSSMSVSVGDRVTIAC | 1-23 | 23 | 51 |
| LFR1 | EIVLTQSPSSMSVSVGDRVTIAC | 1-23 | 23 | 52 |
| CDR-L1 | SASSSVPYMH | 24-33 | 10 | 53 |
| LFR2 | WLQQKPGKSPKLLIY | 34-48 | 15 | 54 |
| CDR-L2 | LTSNLAS | 49-55 | 7 | 55 |
| LFR3 | GVPSRFSGSGSGTDYSLTISSVQPEDAATYYC | 56-87 | 32 | 56 |
| CDR-L3 | QQRSSYPLT | 88-96 | 9 | 39 |
| LFR4 | FGGGTKLEIK | 97-106 | 10 | 40 |

In an embodiment of the invention, the CEA-targeting antibody construct or antigen binding domain comprises the Variable light chain of NP-4/arcitumomab SEQ ID NO. 57

SEQ ID NO. 57
QTVLSQSPAILSASPGEKVTMTCRASSSVTYIHWYQQKPGSSPKSWIYA
TSNLASGVPARFSGSGSGTSYSLTISRVEAEDAATYYCQHWSSKPPTFG
GGTKLEIK

In an embodiment of the invention, the CEA-targeting antibody construct or antigen binding domain comprises the light chain CDR (complementarity determining region) or light chain framework (LFR) sequences of NP-4/arcitumomab SEQ ID NO. 58-64 (full length sequence disclosed as SEQ ID NO: 57).

| Region | Sequence Fragment | Residues | Length | SEQ ID NO. |
|---|---|---|---|---|
| LFR1 | QTVLSQSPAILSASPGEKVTMTC | 1-23 | 23 | 58 |
| CDR-L1 | RASSSVTYIH | 24-33 | 10 | 59 |
| LFR2 | WYQQKPGSSPKSWIY | 34-48 | 15 | 60 |
| CDR-L2 | ATSNLAS | 49-55 | 7 | 61 |
| LFR3 | GVPARFSGSGSGTSYSLTISRVEAEDAATYYC | 56-87 | 32 | 62 |
| CDR-L3 | QHWSSKPPT | 88-96 | 9 | 63 |
| LFR4 | FGGGTKLEIK | 97-106 | 10 | 64 |

In an embodiment of the invention, the CEA-targeting antibody construct or antigen binding domain comprises the Variable heavy chain (VH) of NP-4/arcitumomab SEQ ID NO. 65.

SEQ ID NO. 65
EVKLVESGGGLVQPGGSLRLSCATSGFTFTDYYMNWVRQPPGKALEWLG
FIGNKANGYTTEYSASVKGRFTISRDKSQSILYLQMNTLRAEDSATYYC
TRDRGLRFYFDYWGQGTTLTVSS.

In an embodiment of the invention, the CEA-targeting antibody construct or antigen binding domain comprises the heavy chain CDR (complementarity determining region) or heavy chain framework (HFR) sequences of NP-4 SEQ ID NO. 66-72 (full length sequence disclosed as SEQ ID NO: 65).

| Region | Sequence Fragment | Residues | Length | SEQ ID NO. |
|---|---|---|---|---|
| HFR1 | EVKLVESGGGLVQPGGSLRLSCATSGFTFT | 1-30 | 30 | 66 |
| CDR-H1 | DYYMN | 31-35 | 5 | 67 |
| HFR2 | WVRQPPGKALEWLG | 36-49 | 14 | 68 |
| CDR-H2 | FIGNKANGYTTEYSASVKG | 50-68 | 19 | 69 |
| HFR3 | RFTISRDKSQSILYLQMNTLRAEDSATYYCTR | 69-100 | 32 | 70 |
| CDR-H3 | DRGLRFYFDY | 101-110 | 10 | 71 |
| HFR4 | WGQGTTLTVSS | 111-121 | 11 | 72 |

In an embodiment of the invention, the CEA-targeting antibody construct or antigen binding domain comprises the Variable light chain (VL kappa) of M5A/hT84.66 SEQ ID NO. 73 as disclosed in U.S. Pat. No. 7,776,330, which is incorporated by reference herein for this purpose.

SEQ ID NO. 73
DIQLTQSPSSLSASVGDRVTITCRAGESVDIFGVGELHWYQQKPGKAPK
LLIYRASNLESGVPSRFSGSGSRTDFTLTISSLQPEDFATYYCQQTNED
PYTFGQGTKVEIK

In an embodiment of the invention, the CEA-targeting antibody construct or antigen binding domain comprises the light chain CDR (complementarity determining region) or light chain framework (LFR) sequences of M5A/hT84.66 SEQ ID NO. 74-80 (U.S. Pat. No. 7,776,330) (full length sequence disclosed as SEQ ID NO: 73).

| Region | Sequence Fragment | Residues | Length | SEQ ID NO. |
|---|---|---|---|---|
| LFR1 | DIQLTQSPSSLSASVGDRVTITC | 1-23 | 23 | 74 |
| CDR-L1 | RAGESVDIFGVGFLH | 24-38 | 15 | 75 |
| LFR2 | WYQQKPGKAPKLLIY | 39-53 | 15 | 76 |
| CDR-L2 | RASNLES | 54-60 | 7 | 77 |
| LFR3 | GVPSRFSGSGSRTDFTLTISSLQPEDFATYYC | 61-92 | 32 | 78 |
| CDR-L3 | QQTNEDPYT | 93-101 | 9 | 79 |
| LFR4 | FGQGTKVEIK | 102-111 | 10 | 80 |

In an embodiment of the invention, the CEA-targeting antibody construct or antigen binding domain comprises the Variable heavy chain (VH) of M5A/hT84.66 SEQ ID NO. 81 (U.S. Pat. No. 7,776,330).

SEQ ID NO. 81
EVQLVESGGGLVQPGGSLRLSCAASGFNIKDTYMHWVRQAPGKGLEWVA
RIDPANGNSKYADSVKGRFTISADTSKNTAYLQMNSLRAEDTAVYYCAP
FGYYVSDYAMAYWGQGTLVTVSS

In an embodiment of the invention, the CEA-targeting antibody construct or antigen binding domain comprises the heavy chain CDR (complementarity determining region) or heavy chain framework (HFR) sequences of M5A/hT84.66 SEQ ID NO. 82-88 (U.S. Pat. No. 7,776,330)

| Region | Sequence Fragment | Residues | Length | SEQ ID NO. |
|---|---|---|---|---|
| HFR1 | EVQLVESGGGLVQPGGSLRLSCAASGFNIK | 1-30 | 30 | 82 |
| CDR-H1 | DTYMH | 31-35 | 5 | 83 |
| HFR2 | WVRQAPGKGLEWVA | 36-49 | 14 | 84 |
| CDR-H2 | RIDPANGNSKYADSVKG | 50-66 | 17 | 85 |
| HFR3 | RFTISADTSKNTAYLQMNSLRAEDTAVYYCAP | 67-98 | 32 | 86 |
| CDR-H3 | FGYYVSDYAMAY | 99-110 | 12 | 87 |
| HFR4 | WGQGTLVTVSS | 111-121 | 11 | 88 |

In an embodiment of the invention, the CEA-targeting antibody construct or antigen binding domain comprises the Variable light chain (VL kappa) of hAb2-3 SEQ ID NO. 89 as disclosed in U.S. Pat. No. 9,617,345, which is incorporated by reference herein for this purpose.

SEQ ID NO. 89
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEIK

In an embodiment of the invention, the CEA-targeting antibody construct or antigen binding domain comprises the light chain CDR (complementarity determining region) or light chain framework (LFR) sequences of hAb2-3 SEQ ID NO. 90-96 (U.S. Pat. No. 9,617,345) (full length sequence disclosed as SEQ ID NO: 89).

| Region | Sequence Fragment | Residues | Length | SEQ ID NO. |
|---|---|---|---|---|
| LFR1 | DIQMTQSPASLSASVGDRVTITC | 1-23 | 23 | 90 |
| CDR-L1 | RASENIFSYLA | 24-34 | 11 | 91 |
| LFR2 | WYQQKPGKSPKLLVY | 35-49 | 15 | 92 |
| CDR-L2 | NTRTLAE | 50-56 | 7 | 93 |
| LFR3 | GVPSRFSGSGSGTDFSLTISSLQPEDFATYYC | 57-88 | 32 | 94 |
| CDR-L3 | QHHYGTPFT | 89-97 | 9 | 95 |
| LFR4 | FGSGTKLEIK | 98-107 | 10 | 96 |

In an embodiment of the invention, the CEA-targeting antibody construct or antigen binding domain comprises the Variable heavy chain (VH) of SEQ ID NO. 97 (U.S. Pat. No. 9,617,345).

SEQ ID NO. 97
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSS

In an embodiment of the invention, the CEA-targeting antibody construct or antigen binding domain comprises the heavy chain CDR (complementarity determining region) or heavy chain framework (TER) sequences of hAb2-3 SEQ ID NO. 98-104 (full length sequence disclosed as SEQ ID NO: 97).

| Region | Sequence Fragment | Residues | Length | SEQ ID NO. |
|---|---|---|---|---|
| HFR1 | EVQLQESGPGLVKPGGSLSLSCAASGFVFS | 1-30 | 30 | 98 |
| CDR-H1 | SYDMS | 31-35 | 5 | 99 |
| HFR2 | WVRQTPERGLEWVA | 36-49 | 14 | 100 |
| CDR-H2 | YISSGGGITYAPSTVKG | 50-66 | 17 | 101 |
| HFR3 | RFTVSRDNAKNTLYLQMNSLTSEDTAVYYCAA | 67-98 | 32 | 102 |
| CDR-H3 | HYFGSSGPFAY | 99-109 | 11 | 103 |
| HFR4 | WGQGTLVTVSS | 110-120 | 11 | 104 |

In an embodiment of the invention, the CEA-targeting antibody construct or antigen binding domain comprises the Variable light chain (VL kappa) of A240VL-B9VH/AMG-211 SEQ ID NO. 105 as disclosed in U.S. Pat. No. 9,982,063, which is incorporated by reference herein for this purpose.

SEQ ID NO. 105
QAVLTQPASLSASPGASASLTCTLRRGINVGAYSIYWYQQKPGSPPQYL
LRYKSDSDKQQGSGVSSRFSASKDASANAGILLISGLQSEDEADYYCMI
WHSGASAVFGGGTKLTVL

In an embodiment of the invention, the CEA-targeting antibody construct or antigen binding domain comprises the light chain CDR (complementarity determining region) or light chain framework (LFR) sequences of A240VL-B9VH/AMG-211 SEQ ID NO. 106-112 (U.S. Pat. No. 9,982,063) (full length sequence disclosed as SEQ ID NO: 105).

| Region | Sequence Fragment | Residues | Length | SEQ ID NO. |
|---|---|---|---|---|
| LFR1 | QAVLTQPASLSASPGASASLTC | 1-22 | 22 | 106 |
| CDR-L1 | TLRRGINVGAYSIY | 23-36 | 14 | 107 |
| LFR2 | WYQQKPGSPPQYLLR | 37-51 | 15 | 108 |
| CDR-L2 | YKSDSDKQQGS | 52-62 | 11 | 109 |
| LFR3 | GVSSRFSASKDASANAGILLISGLQSEDEADYYC | 63-96 | 34 | 110 |
| CDR-L3 | MIWHSGASAV | 97-106 | 10 | 111 |
| LFR4 | FGGGTKLTVL | 107-116 | 10 | 112 |

In an embodiment of the invention, the CEA-targeting antibody construct or antigen binding domain comprises the Variable heavy chain (VH) of B9VH SEQ ID NO. 113 (U.S. Pat. No. 9,982,063).

SEQ ID NO. 113
EVQLVESGGGLVQPGRSLRLSCAASGFTVSSYWMHWVRQAPGKGLEWVG
FIRNKANGGTTEYAASVKGRFTISRDDSKNTLYLQMNSLRAEDTAVYYC
ARDRGLRFYFDYWGQGTTVTVSS

In an embodiment of the invention, the CEA-targeting antibody construct or antigen binding domain comprises the heavy chain CDR (complementarity determining region) or heavy chain framework (HER) sequences of SEQ ID NO. 114-121 (U.S. Pat. No. 9,982,063). The embodiment includes two variants of CDR-H2, SEQ ID NO.: 117 and SEQ ID NO.: 118 (full length sequences disclosed as SE ID NOS 113 and 175, respectively in order of appearance).

| Region | Sequence Fragment | Residues | Length | SEQ ID NO. |
|---|---|---|---|---|
| HFR1 | EVQLVESGGGLVQPGRSLRLSCAASGFTVS | 1-30 | 30 | 114 |
| CDR-H1 | SYWMH | 31-35 | 5 | 115 |
| HFR2 | WVRQAPGKGLEWVG | 36-49 | 14 | 116 |
| CDR-H2 | FIRNKANGGTTEYAASVKG | 50-68 | 19 | 117 |
| CDR-H2 | FIRNKANSGTTEYAASVKG | 50-68 | 19 | 118 |
| HFR3 | RFTISRDDSKNTLYLQMNSLRAEDTAVYYCAR | 69-100 | 32 | 119 |
| CDR-H3 | DRGLRFYFDY | 101-110 | 10 | 120 |
| HFR4 | WGQGTTVTVSS | 111-121 | 11 | 121 |

In an embodiment of the invention, the CEA-targeting antibody construct or antigen binding domain comprises the Variable heavy chain (VH) of E12VH SEQ ID NO. 122 (U.S. Pat. No. 9,982,063).

SEQ ID NO. 122
EVQLVESGGGLVQPGRSLRLSCAASGFTVSSYWMHWVRQAPGKGLEWVG
FILNKANGGTTEYAASVKGRFTISRDDSKNTLYLQMNSLRAEDTAVYYC
ARDRGLRFYFDYWGQGTTVTVSS

In an embodiment of the invention, the CEA-targeting antibody construct or antigen binding domain comprises the heavy chain CDR (complementarity determining region) or heavy chain framework (HFR) sequences of SEQ ID NO. 123-129 (U.S. Pat. No. 9,982,063) (full length sequence disclosed as SE ID NO: 122).

| Region | Sequence Fragment | Residues | Length | SEQ ID NO. |
|---|---|---|---|---|
| HFR1 | EVQLVESGGGLVQPGRSLRLSCAASGFTVS | 1-30 | 30 | 123 |
| CDR-H1 | SYWMH | 31-35 | 5 | 124 |
| HFR2 | WVRQAPGKGLEWVG | 36-49 | 14 | 125 |
| CDR-H2 | FILNKANGGTTEYAASVKG | 50-68 | 19 | 126 |
| HFR3 | RFTISRDDSKNTLYLQMNSLRAEDTAVYYCAR | 69-100 | 32 | 127 |
| CDR-H3 | DRGLRFYFDY | 101-110 | 10 | 128 |
| HFR4 | WGQGTTVTVSS | 111-121 | 11 | 129 |

In an embodiment of the invention, the CEA-targeting antibody construct or antigen binding domain comprises the Variable heavy chain (VH) of PR1A3 VH SEQ ID NO. 130 (U.S. Pat. No. 8,642,742).

SEQ ID NO. 130
QVQLVQSGAEVKKPGASVKVSCKASGYTFTEFGMNWVRQAPGQGLEWMG
WINTKTGEATYVEEFKGRVTFTTDTSTSTAYMELRSLRSDDTAVYYCAR
WDFAYYVEAMDYWGQGTTVTVSS

In an exemplary embodiment, the immunoconjugates of the invention comprise an antibody construct that comprises an antigen binding domain that specifically recognizes and binds TROP2.

Tumor-associated calcium signal transducer 2 (TROP2) is a transmembrane glycoprotein encoded by the TACSTD2 gene (Linnenbach A J, et al (1993) Mol Cell Biol. 13(3): 1507-15; Calabrese G, et al (2001) Cytogenet Cell Genet. 92(1-2): 164-5). It is an intracellular calcium signal transducer that is differentially expressed in many cancers. It signals cells for self-renewal, proliferation, invasion, and survival. It has stem cell-like qualities. TROP2 is expressed in many normal tissues, though in contrast, it is overexpressed in many cancers (Ohmachi T, et al., (2006) Clin. Cancer Res., 12(10), 3057-3063; Muhlmann G, et al., (2009) J. Clin. Pathol., 62(2), 152-158; Fong D, et al., (2008) Br. J. Cancer, 99(8), 1290-1295; Fong D, et al., (2008) Mod. Pathol., 21(2), 186-191; Ning S, et al., (2013) Neurol. Sci., 34(10), 1745-1750). Overexpression of TROP2 is of prognostic significance. Several ligands have been proposed that interact with TROP2, TROP2 signals the cells via different pathways and it is transcriptionally regulated by a complex network of several transcription factors.

Human TROP2 (TACSTD2: tumor-associated calcium signal transducer 2, GA733-1, EGP-1, M1S1; hereinafter, referred to as hTROP2) is a single-pass transmembrane type 1 cell membrane protein consisting of 323 amino acid residues. While the presence of a cell membrane protein involved in immune resistance, which is common to human trophoblasts and cancer cells (Faulk W P, et al. (1978), Proc. Natl. Acad. Sci. 75(4):1947-1951), has previously been suggested, an antigen molecule recognized by a monoclonal antibody against a cell membrane protein in a human choriocarcinoma cell line was identified and designated as TROP2 as one of the molecules expressed in human trophoblasts (Lipinski M, et al. (1981), Proc. Natl. Acad. Sci. 78(8), 5147-5150). This molecule was also designated as tumor antigen GA733-1 recognized by a mouse monoclonal antibody GA733 (Linnenbach A J, et al., (1989) Proc. Natl. Acad. Sci. 86(1), 27-31) obtained by immunization with a gastric cancer cell line or an epithelial glycoprotein (EGP-1; Basu A, et al., Int. J Cancer, 62 (4), 472-479 (1995)) recognized by a mouse monoclonal antibody RS7-3G11 obtained by immunization with non-small cell lung cancer cells. In 1995, however, the TROP2 gene was cloned, and all of these molecules were confirmed to be identical molecules (Fornaro M, et al., (1995) Int. J. Cancer, 62(5), 610-618). The DNA sequence and amino acid sequence of hTROP2 are available on a public database and can be referred to, for example, under Accession Nos. NM_002353 and NP_002344 (NCBI).

In response to such information suggesting the association with cancer, a plurality of anti-hTROP2 antibodies have been established so far and studied for their antitumor effects.

Among these antibodies, there is disclosed, for example, an unconjugated antibody that exhibits in itself antitumor activity in nude mouse xenograft models (WO 2008/144891; WO 2011/145744; WO 2011/155579; WO 2013/077458) as well as an antibody that exhibits antitumor activity as ADC with a cytotoxic drug (WO 2003/074566; WO 2011/068845; WO 2013/068946; U.S. Pat. No. 7,999,083). However, the strength or coverage of their activity is still insufficient, and there are unsatisfied medical needs for hTROP2 as a therapeutic target.

TROP2 expression in cancer cells has been correlated with drug resistance. Several strategies target TROP2 on cancer cells that include antibodies, antibody fusion proteins, chemical inhibitors, nanoparticles, etc. The in vitro studies and pre-clinical studies, using these various therapeutic treatments, have resulted in significant inhibition of tumor cell growth both in vitro and in vivo in mice. Clinical studies have explored the potential application of Trop2 as both a prognostic biomarker and as a therapeutic target to reverse resistance.

Sacituzumab govitecan (TRODELVY®, Immunomedics, IMMU-132), an antibody-drug conjugate comprising a TROP2-directed antibody linked to a topoisomerase inhibitor drug, is indicated for the treatment of metastatic triple-negative breast cancer (mTNBC) in adult patients that have received at least two prior therapies. The TROP2 antibody in sacituzumab govitecan is conjugated to SN-38, the active metabolite of irinotecan (US 2016/0297890; WO 2015/098099).

In an embodiment of the invention, the TROP2-targeting antibody construct or antigen binding domain comprises the light chain CDR (complementarity determining region) sequences of hRS7 (humanized RS7), SEQ ID NO:131-133 (U.S. Pat. Nos. 7,238,785; 7,420,040 incorporated by reference herein).

| Region | CDR Sequence Fragment | SEQ ID NO: |
|---|---|---|
| CDR-L1 | KASQDVSIAVA | 131 |
| CDR-L2 | SASYRYT | 132 |
| CDR-L3 | QQHYITPLT | 133 |

In an embodiment of the invention, the TROP2-targeting antibody construct or antigen binding domain comprises the heavy chain CDR (complementarity determining region) sequences of hRS7 (humanized RS7), SEQ ID NO:134-136 (U.S. Pat. Nos. 7,238,785; 9,797,907; 9,382,329; WO 2020/142659, each incorporated by reference herein).

| Region | CDR Sequence Fragment | SEQ ID NO: |
|---|---|---|
| CDR-H1 | NYGMN | 134 |
| CDR-H2 | WINTYTGEPTYTDDFKG | 135 |
| CDR-H3 | GGFGSSYWYFDV | 136 |

In an embodiment of the invention, the TROP2-targeting antibody construct or antigen binding domain comprises the heavy chain CDR (complementarity determining region) sequences of AR47A6.4.2, SEQ ID NO:134, 137, 138 (U.S. Pat. No. 7,420,040, incorporated by reference herein).

| Region | CDR Sequence Fragment | SEQ ID NO: |
|---|---|---|
| CDR-H1 | NYGMN | 134 |
| CDR-H2 | WINTKTGEPTYAEEFKG | 137 |
| CDR-H3 | GGYGSSYWYFDV | 138 |

In an embodiment of the invention, the TROP2-targeting antibody construct or antigen binding domain comprises the light chain CDR (complementarity determining region) sequences of humanized KM4097, SEQ ID NO:139-141 (US 2012/0237518, incorporated by reference herein).

| Region | CDR Sequence Fragment | SEQ ID NO: |
|---|---|---|
| CDR-L1 | KSSQSLLNSGNQQNYLA | 139 |
| CDR-L2 | GASTRES | 140 |
| CDR-L3 | QSDHIYPYT | 141 |

In an embodiment of the invention, the TROP2-targeting antibody construct or antigen binding domain comprises the heavy chain CDR (complementarity determining region) sequences of humanized KM4097, SEQ ID NO:142-144 (US 2012/0237518, incorporated by reference herein).

| Region | CDR Sequence Fragment | SEQ ID NO: |
|---|---|---|
| CDR-H1 | IYWLG | 142 |
| CDR-H2 | NIFPGSAYINYNEKFKG | 143 |
| CDR-H3 | EGSNSGY | 144 |

In an embodiment of the invention, the TROP2-targeting antibody construct or antigen binding domain comprises the light chain CDR (complementarity determining region) sequences of hTINA1-H1L1, SEQ ID NO: 132, 133, 145 (U.S. Pat. No. 10,227,417, incorporated by reference herein).

| Region | CDR Sequence Fragment | SEQ ID NO: |
|---|---|---|
| CDR-L1 | KASQDVSTAVA | 145 |
| CDR-L2 | SASYRYT | 132 |
| CDR-L3 | QQHYITPLT | 133 |

In an embodiment of the invention, the TROP2-targeting antibody construct or antigen binding domain comprises the heavy chain CDR (complementarity determining region) sequences of hTINA1-H1L1, SEQ ID NO:146-148 (U.S. Pat. No. 10,227,417, incorporated by reference herein).

| Region | CDR Sequence Fragment | SEQ ID NO: |
|---|---|---|
| CDR-H1 | TAGMQ | 146 |
| CDR-H2 | WINTHSGVPKYAEDFKG | 147 |
| CDR-H3 | SGFGSSYWYFDV | 148 |

In an embodiment of the invention, the TROP2-targeting antibody construct or antigen binding domain comprises the light chain CDR (complementarity determining region) sequences of hTINA1-H1L1, SEQ ID NO: 149-151 (U.S. Pat. No. 8,871,908, incorporated by reference herein).

| Region | CDR Sequence Fragment | SEQ ID NO: |
|---|---|---|
| CDR-L1 | RASKSVSTS(X1)YSYMH | 149 |
| | where X1 is G, L, or N | |
| CDR-L2 | LASNLES | 150 |
| CDR-L3 | QHSRELPYT | 151 |

In an embodiment of the invention, the TROP2-targeting antibody construct or antigen binding domain comprises the heavy chain CDR (complementarity determining region) sequences of hTINA1-H1L1, SEQ ID NO: 152-157 (U.S. Pat. No. 8,871,908, incorporated by reference herein).

| Region | CDR Sequence Fragment | SEQ ID NO: |
|---|---|---|
| CDR-H1 | SYGVH | 152 |
| CDR-H1 | GGSISSY | 153 |
| CDR-H1 | GGSISSYGVH | 154 |
| CDR-H2 | VIWT(X1)G(X2)TDYNSALM(X3) | 155 |
| | where X1 is G or S; X2 is S or V; X3 is | |
| | S or G | |
| CDR-H2 | WT(X1)G(X2) | 156 |
| | where X1 is G or S; X2 is S or V | |
| CDR-H3 | DGDYDRYTMDY | 157 |

In an embodiment of the invention, the TROP2-targeting antibody construct or antigen binding domain comprises the light chain CDR (complementarity determining region) sequences SEQ ID NO: 150, 151, 158 of hTINA1-H1L1, (U.S. Pat. No. 8,871,908, incorporated by reference herein).

| Region | CDR Sequence Fragment | SEQ ID NO: |
|---|---|---|
| CDR-L1 | RASKSVSTSGYSYMH | 158 |
| CDR-L2 | LASNLES | 150 |
| CDR-L3 | QHSRELPYT | 151 |

In an embodiment of the invention, the TROP2-targeting antibody construct or antigen binding domain comprises the heavy chain CDR (complementarity determining region) sequences SEQ ID NO: 152-154, 157, 159, 160 of hTINA1-H1L1, (U.S. Pat. No. 8,871,908, incorporated by reference herein).

| Region | CDR Sequence Fragment | SEQ ID NO: |
|---|---|---|
| CDR-H1 | SYGVH | 152 |
| CDR-H1 | GGSISSY | 153 |
| CDR-H1 | GGSISSYGVH | 154 |
| CDR-H2 | VIWTSGVTDYNSALMG | 159 |
| CDR-H2 | WTSGV | 160 |
| CDR-H3 | DGDYDRYTMDY | 157 |

In an embodiment of the invention, an immunoconjugate comprises a cysteine-mutant, antibody with a light chain sequence selected from SEQ ID NO: 161-163.

| Sequence: | mutant site | SEQ ID NO: |
|---|---|---|
| KADYECHKVYA | LC K188C | 161 |
| YEKHKCYACEV | LC V191C | 162 |
| QLKSGCASVVC | LC T129C | 163 |

In an embodiment of the invention, a cysteine-mutant, TROP2-targeting antibody comprises the heavy chain (HC) of SEQ ID NO: 164.

SEQ ID NO. 164
QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

In an embodiment of the invention, the light chain (LC) of a TROP2-targeting antibody is selected from SEQ ID NO: 165-167.

| | Cys Mutant | SEQ ID |
|---|---|---|
| Heavy chain | site | NO: |
| DIQLTQSPSSLSASVGDRVSITCKASQDVSIAVAWYQQKPGKAPKLLI | LC K188C | 165 |
| YSASYRYTGVPDRFSGSGSGTDFTLTISSLQPEDFAVYYCQQHYITPL | | |
| TFGAGTKVEIKRTVAAPSVFIFPPSDEQLKSGTASVVCLLNNFYPREA | | |
| KVQWKVDNALQSGNSQESVTEQDSKDSTYSLSSTLTLSKADYECHK | | |
| VYACEVTHQGLSSPVTKSFNRGEC | | |
| DIQLTQSPSSLSASVGDRVSITCKASQDVSIAVAWYQQKPGKAPKLLI | LC V191C | 166 |
| YSASYRYTGVPDRFSGSGSGTDFTLTISSLQPEDFAVYYCQQHYITPL | | |
| TFGAGTKVEIKRTVAAPSVFIFPPSDEQLKSGTASVVCLLNNFYPREA | | |
| KVQWKVDNALQSGNSQESVTEQDSKDSTYSLSSTLTLSKADYEKHK | | |
| CYACEVTHQGLSSPVTKSFNRGEC | | |
| DIQLTQSPSSLSASVGDRVSITCKASQDVSIAVAWYQQKPGKAPKLLI | LC T129C | 167 |
| YSASYRYTGVPDRFSGSGSGTDFTLTISSLQPEDFAVYYCQQHYITPL | | |
| TFGAGTKVEIKRTVAAPSVFIFPPSDEQLKSGCASVVCLLNNFYPREA | | |
| KVQWKVDNALQSGNSQESVTEQDSKDSTYSLSSTLTLSKADYEKHK | | |
| VYACEVTHQGLSSPVTKSFNRGEC | | |

In an embodiment of the invention, an immunoconjugate comprises a cysteine-mutant, antibody with a heavy chain sequence of SEQ ID NO: 168.

| | Cys mutant | SEQ ID |
|---|---|---|
| Sequence: | site | NO: |
| TVSSACTKGPS | HC S119C | 168 |

In an embodiment of the invention, the light chain (LC) of a cysteine-mutant, TROP2-targeting antibody has the sequence of SEQ ID NO: 169.

SEQ ID NO: 169
DIQLTQSPSSLSASVGDRVSITCKASQDVSIAVAWYQQKPGKAPKLLIY
SASYRYTGVPDRFSGSGSGTDFTLTISSLQPEDFAVYYCQQHYITPLTF
GAGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In an embodiment of the invention, the heavy chain (HC) of a cysteine-mutant, TROP2-targeting antibody has the sequence of SEQ ID NO: 170.

SEQ ID NO: 170
QVQLVQSGAEVKKPGASVKVSCKASGDTFTNHYMHWVRQAPGQGLEWMG
WINPNSGHTGYAQKFQGRVTMTRDTSTSTVYMELSSLRSEDTAVYYCAR
EAVAGPMDVWGQGTTVTVSSACTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

In some embodiments, the antibody construct further comprises an Fc domain. In certain embodiments, the antibody construct is an antibody. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an anti-PD-L1 antibody, an anti-HER2 antibody, or an anti-CEA antibody, each variable region comprising a CDR1, a CDR2, and a CDR3.

In some embodiments, the antibodies in the immunoconjugates contain a modified Fc region, wherein the modification modulates the binding of the Fc region to one or more Fc receptors.

In some embodiments, the Fc region is modified by inclusion of a transforming growth factor beta 1 (TGFβ1) receptor, or a fragment thereof, that is capable of binding TGFβ1. For example, the receptor can be TGFβ receptor II (TGFβRII). In some embodiments, the TGFβ receptor is a human TGFβ receptor. In some embodiments, the IgG has a C-terminal fusion to a TGFβRII extracellular domain (ECD) as described in U.S. Pat. No. 9,676,863, incorporated herein. An “Fc linker” may be used to attach the IgG to the TGFβRII extracellular domain, for example, a G₄S₄G Fc linker (SEQ ID NO: 171). The Fc linker may be a short, flexible peptide that allows for the proper three-dimensional folding of the molecule while maintaining the binding-specificity to the targets. In some embodiments, the N-terminus of the TGFβ receptor is fused to the Fc of the antibody construct (with or without an Fc linker). In some embodiments, the C-terminus of the antibody construct heavy chain is fused to the TGFβ receptor (with or without an Fc linker). In some embodiments, the C-terminal lysine residue of the antibody construct heavy chain is mutated to alanine.

In some embodiments, the antibodies in the immunoconjugates are glycosylated.

In some embodiments, the antibody of the immunoconjugate is a cysteine-engineered antibody which provides for site-specific conjugation of an adjuvant to the antibody through cysteine substitutions at sites where the engineered cysteines are available and reactive for conjugation but do not perturb immunoglobulin folding and assembly or alter antigen binding and effector functions (Junutula, et al., 2008b Nature Biotech., 26(8):925-932; Dornan et al. (2009) Blood 114(13):2721-2729; U.S. Pat. Nos. 7,521,541; 7,723,485; US 2012/0121615; WO 2009/052249). A “cysteine engineered antibody” or “cysteine engineered antibody variant” is an antibody in which one or more residues of an antibody are substituted with cysteine residues.

Cysteine-engineered antibodies can be conjugated to a thiol-reactive electrophilic group such as maleimide on the 2-amino-4-carboxamide-benzazepine-linker compound (Formula II) with uniform stoichiometry (e.g., up to two 2-amino-4-carboxamide-benzazepinemoieties per antibody in an antibody that has a single engineered cysteine site).

In some embodiments, cysteine-engineered antibodies are used to prepare immunoconjugates. Immunoconjugates may have a reactive cysteine thiol residue introduced at a site on the light chain, such as the 149-lysine site (LC K149C), or on the heavy chain such as the 122-serine site (HC S122C), as numbered by Kabat numbering. In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced in: (i) the light chain at G64C, R142C, K188C, L201C, T129C, S 114C, or E105C according to Kabat numbering; (ii) the heavy chain at D101C, V184C, T205C, or S122C according to Kabat numbering; or (iii) other cysteine-mutant antibodies, and as described in Bhakta, S. et al, (2013) “Engineering THIOMABs for Site-Specific Conjugation of Thiol-Reactive Linkers”, Laurent Ducry (ed.), Antibody-Drug Conjugates, Methods in Molecular Biology, vol. 1045, pages 189-203; WO 2011/156328; U.S. Pat. No. 9,000,130.

### 2-Amino-4-Carboxamide-Benzazepine Adjuvant Compounds

The immunoconjugate of the invention comprises a 2-amino-4-carboxamide-benzazepine adjuvant moiety. The adjuvant moiety described herein is a compound that elicits an immune response (i.e., an immunostimulatory agent). Generally, the adjuvant moiety described herein is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-κB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

Exemplary compounds (2Am4CBza) of the invention are shown in Table 1. Each compound was characterized by mass spectrometry and shown to have the mass indicated.

Activity against HEK293 NFKB reporter cells expressing human TLR7 or human TLR8 was measured according to Example 203.

TABLE 1
2-amino-4-carboxamide-benzazepine compounds (2Am4CBza)

| 2Am4CBza | | |
|---|---|---|
| No. | Structure | MW |
| 2Am4CBza-1 | 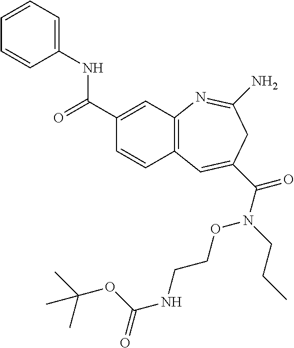 [SMILES: CCCN(OCCNC(=O)OC(C)(C)C)C(=O)C1=Cc2ccc(C(=O)Nc3ccccc3)cc2N=C(N)C1] | 521.6 |

### 2-Amino-4-Carboxamide-Benzazepine-Linker Compounds

The immunoconjugates of the invention are prepared by conjugation of an antibody with a 2-amino-4-carboxamide-benzazepine-linker compound. The 2-amino-4-carboxamide-benzazepine-linker compounds comprise a 2-amino-4-carboxamide-benzazepine (2Am4CBza) moiety covalently attached to a linker unit, L and a reactive electrophilic group, Q. The linker units comprise functional groups and subunits which affect stability, permeability, solubility, and other pharmacokinetic, safety, and efficacy properties of the immunoconjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the 2Am4CBza-linker compound to form the immunoconjugate (IC). Also, for example, a cysteine thiol of the antibody reacts with a maleimide or bromoacetamide group of the 2Am4CBza-linker compound to form the immunoconjugate.

Reactive electrophilic functional groups (Q in Formula II) suitable for the 2Am4CBza-linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP), or sulfotetrafluorophenyl (SulfoTFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2nd Edition, Academic Press, 2008.

The invention provides solutions to the limitations and challenges to the design, preparation and use of immunoconjugates. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Exemplary embodiments of a 2-amino-4-carboxamide-benzazepine-linker compound includes Formula II:

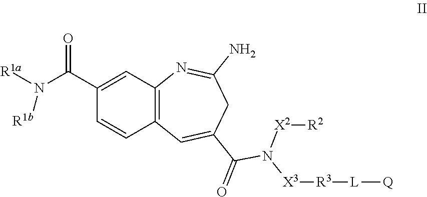

wherein
X² and X³ are independently selected from the group consisting of a bond, C(═O), C(═O)N(R⁵), O, N(R⁵), S, S(O)₂, and S(O)₂N(R⁵);
R^{1a}, R^{1b}, and R² are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl;
R³ is selected from the group consisting of:
—(C₁-C₁₂ alkyldiyl)-N(R⁵)—*;
—(C₁-C₁₂ alkyldiyl)-N(R⁵)—C(═O)*;
—(C₁-C₁₂ alkyldiyl)-N(R⁵)—C(═O)O—(C₃-C₁₂ carbocyclyldiyl)-*;
—(C₁-C₁₂ alkyldiyl)-N(R⁵)—(C₁-C₂₀ heteroaryldiyl)-*;
—(C₁-C₁₂ alkyldiyl)-N(R⁵)—(C₁-C₂₀ heteroaryldiyl)-(C₁-C₁₂ alkyldiyl)-*;
—(C₁-C₁₂ alkyldiyl)-N(R⁵)—S(O₂)—*;
—(C₁-C₁₂ alkyldiyl)-OC(═O)—(C₂-C₉ heterocyclyldiyl)-*;
—(C₁-C₁₂ alkyldiyl)-O—*;
—(C₁-C₁₂ alkyldiyl)-(C₃-C₁₂ carbocyclyldiyl)-*;
—(C₁-C₁₂ alkyldiyl)-(C₆-C₂₀ aryldiyl)-*;
—(C₁-C₁₂ alkyldiyl)-(C₆-C₂₀ aryl)-(C₁-C₁₂ alkyldiyl)-N(R⁵)—*;
—(C₁-C₁₂ alkyldiyl)-(C₆-C₂₀ aryl)-(C₁-C₁₂ alkyldiyl)-N(R⁵)—*;
—(C₁-C₁₂ alkyldiyl)-(C₂-C₉ heterocyclyldiyl)-(C₁-C₁₂ alkyldiyl)-N(R⁵)—*;
—(C₁-C₁₂ alkyldiyl)-(C₁-C₂₀ heteroaryldiyl)-N(R⁵)—*;
—(C₁-C₁₂ alkyldiyl)-(C₁-C₂₀ heteroaryldiyl)-*;
—(C₁-C₁₂ alkyldiyl)-(C₁-C₂₀ heteroaryldiyl)-(C₁-C₁₂ alkyldiyl)-*;
—(C₁-C₁₂ alkyldiyl)-(C₁-C₂₀ heteroaryldiyl)-(C₁-C₁₂ alkyldiyl)-N(R⁵)—*;
—(C₃-C₁₂ carbocyclyldiyl)-*;
—(C₃-C₁₂ carbocyclyldiyl)-(C₁-C₁₂ alkyldiyl)-N(R⁵)—*;
—(C₃-C₁₂ carbocyclyldiyl)-(C₁-C₁₂ alkyldiyl)-N(R⁵)—*;
—(C₃-C₁₂ carbocyclyldiyl)-NR⁵—C(═NR^{5a})—N(R⁵)—*;
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)—C(═NR^{5a})—N(R⁵)—*;
—(C₂-C₂₀ heterocyclyldiyl)-*;
—(C₂-C₉ heterocyclyldiyl)-(C₁-C₁₂ alkyldiyl)-N(R⁵)—*;
—(C₂-C₉ heterocyclyldiyl)-N(R⁵)—C(═NR^{5a})—N(R⁵)—*;
—(C₁-C₂₀ heteroaryldiyl)-*;
—(C₁-C₂₀ heteroaryldiyl)-(C₁-C₁₂ alkyldiyl)-N(R⁵)—*;
—(C₁-C₂₀ heteroaryldiyl)-(C₁-C₁₂ alkyldiyl)-O—*; and
—(C₁-C₂₀ heteroaryldiyl)-N(R⁵)—C(═NR^{5A})—N(R⁵)—*;
where the asterisk * indicates the attachment site of the linker L;
R⁵ is selected from the group consisting of H, C₆-C₂₀ aryl and C₁-C₁₂ alkyl, or two R⁵ groups together form a 5- or 6-membered heterocyclyl ring;
R^{5a} is selected from the group consisting of C₆-C₂₀ aryl and C₁-C₂₀ heteroaryl;
L-Q is selected from the group consisting of:
   Q-C(═O)-PEG-;
   Q-C(═O)-PEG-C(═O)N(R⁶)—(C₁-C₁₂ alkyldiyl)-C(═O)-Gluc-;
   Q-C(═O)-PEG-O—;
   Q-C(═O)-PEG-O—C(═O)—;
   Q-C(═O)-PEG-C(═O)—;
   Q-C(═O)-PEG-C(═O)-PEP-;
   Q-C(═O)-PEG-N(R⁶)—;
   Q-C(═O)-PEG-N(R⁶)—C(═O)—;
   Q-C(═O)-PEG-N(R⁶)-PEG-C(═O)-PEP-;
   Q-C(═O)-PEG-N⁺(R⁶)₂-PEG-C(═O)-PEP-;
   Q-C(═O)-PEG-C(═O)-PEP-N(R⁶)—(C₁-C₁₂ alkyldiyl)-;
   Q-C(═O)-PEG-C(═O)-PEP-N(R⁶)—(C₁-C₁₂ alkyldiyl)N(R⁶)C(═O)—(C₂-C₅ monoheterocyclyldiyl)-;
   Q-C(═O)-PEG-SS-(C₁-C₁₂ alkyldiyl)-OC(═O)—;
   Q-C(═O)-PEG-SS-(C₁-C₂ alkyldiyl)-C(═O)—;
   Q-C(═O)—(C₁-C₁₂ alkyldiyl)-C(═O)-PEP-;
   Q-C(═O)—(C₁-C₁₂ alkyldiyl)-C(═O)-PEP-N(R⁶)—(C₁-C₁₂ alkyldiyl)-;
   Q-C(═O)—(C₁-C₁₂ alkyldiyl)-C(═O)-PEP-N(R⁶)—(C₁-C₂ alkyldiyl)-N(R⁵)—C(═O);
Q-C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
   Q-(CH₂)ₘ—C(═O)N(R⁶)-PEG-;
   Q-(CH₂)ₘ—C(═O)N(R⁶)-PEG-C(═O)N(R⁶)—(C₁-C₁₂ alkyldiyl)-C(═O)-Gluc-;
   Q-(CH₂)ₘ—C(═O)N(R⁶)-PEG-O—;
   Q-(CH₂)ₘ—C(═O)N(R⁶)-PEG-O—C(═O)—;
   Q-(CH₂)ₘ—C(═O)N(R⁶)-PEG-C(═O)—;
   Q-(CH₂)ₘ—C(═O)N(R⁶)-PEG-N(R⁵)—;
   Q-(CH₂)ₘ—C(═O)N(R⁶)-PEG-N(R⁵)—C(═O)—;
   Q-(CH₂)ₘ—C(═O)N(R⁶)-PEG-C(═O)-PEP-;
   Q-(CH₂)ₘ—C(═O)N(R⁶)-PEG-SS-(C₁-C₁₂ alkyldiyl)-OC(═O)—;
   Q-(CH₂)ₘ—C(═O)-PEP-N(R⁶)—(C₁-C₁₂ alkyldiyl)-;
   Q-(CH₂)ₘ—C(═O)-PEP-N(R⁶)—(C₁-C₁₂ alkyldiyl)N(R⁶)C(═O)—; and
   Q-(CH₂)ₘ—C(═O)-PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ— where m is an integer from 1 to 5, and n is an integer from 2 to 50;
Gluc has the formula:

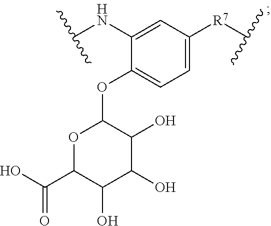

PEP has the formula:

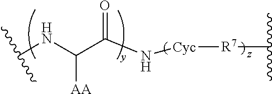

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl , NO₂, —OH, —OCH₃, and a glucuronic acid having the structure:

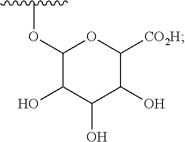 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1;
Q is selected from the group consisting of N-hydroxysuccinimidyl, N-hydroxysulfosuccinimidyl, maleimide, and phenoxy substituted with one or more groups independently selected from F, Cl , NO₂, and SO₃⁻; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —C_{H}═CH₂, —C≡C_{H}, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH(OH)CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the 2-amino-4-carboxamide-benzazepine-linker compound of Formula II includes wherein R^{1a} and R^{1b} are independently selected from a group consisting of optionally substituted C₆-C₂₀ aryl, C₂-C₉ heterocyclyl, and C₁-C₂₀ heteroaryl.

An exemplary embodiment of the 2-amino-4-carboxamide-benzazepine-linker compound of Formula II includes wherein R^{1a} is optionally substituted C₆-C₂₀ aryl and R^{1b} is H.

An exemplary embodiment of the 2-amino-4-carboxamide-benzazepine-linker compound of Formula II includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

An exemplary embodiment of the 2-amino-4-carboxamide-benzazepine-linker compound of Formula II includes wherein X² is a bond, and R² is C₁-C₁₂ alkyl.

An exemplary embodiment of the 2-amino-4-carboxamide-benzazepine-linker compound of Formula II includes wherein X³ is 0 and R³ is —(C₁-C₁₂ alkyldiyl)-N(R⁵)—*.

An exemplary embodiment of the 2-amino-4-carboxamide-benzazepine-linker compound of Formula II includes wherein R³ is —CH₂CH₂CH₂NH—.

An exemplary embodiment of the 2-amino-4-carboxamide-benzazepine-linker compound of Formula II includes wherein L is —C(═O)-PEG-C(═O)—.

An exemplary embodiment of the 2-amino-4-carboxamide-benzazepine-linker compound of Formula II includes wherein AA₁ and AA₂ are independently selected from a side chain of a naturally-occurring amino acid.

An exemplary embodiment of the 2-amino-4-carboxamide-benzazepine-linker compound of Formula II includes wherein AA₁ or AA₂ with an adjacent nitrogen atom form a 5-membered ring proline amino acid.

An exemplary embodiment of the 2-amino-4-carboxamide-benzazepine-linker compound of Formula II includes wherein AA₁ and AA₂ are independently selected from H, —CH₃, —CH(CH₃)₂, —CH₂(C₆H5), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

An exemplary embodiment of the 2-amino-4-carboxamide-benzazepine-linker compound of Formula II includes wherein AA₁ is —CH(CH₃)₂, and AA₂ is —CH₂CH₂CH₂NHC(O)NH₂.

An exemplary embodiment of the 2-amino-4-carboxamide-benzazepine-linker compound of Formula II includes wherein Q is selected from:

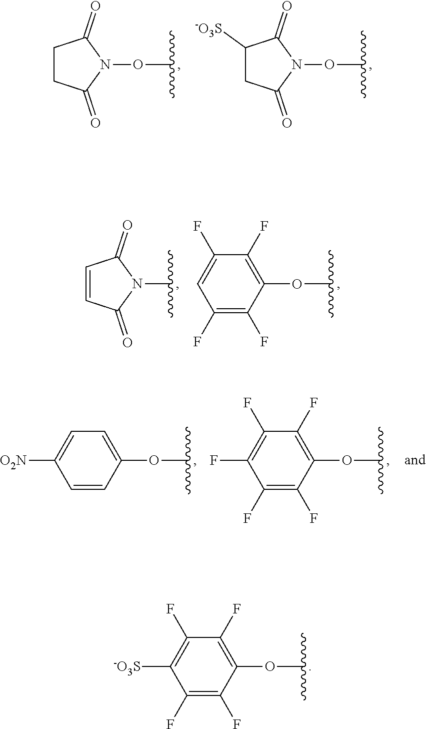

An exemplary embodiment of the 2-amino-4-carboxamide-benzazepine-linker compound of Formula II includes wherein Q is phenoxy substituted with one or more F.

An exemplary embodiment of the 2-amino-4-carboxamide-benzazepine-linker compound of Formula II includes wherein Q is 2,3,5,6-tetrafluorophenoxy.

An exemplary embodiment of the 2-amino-4-carboxamide-benzazepine-linker compound of Formula II includes wherein Q is 2,3,5,6-tetrafluoro, 4-sulfonate-phenoxy.

An exemplary embodiment of the 2-amino-4-carboxamide-benzazepine-linker compound of Formula II has Formula IIa:

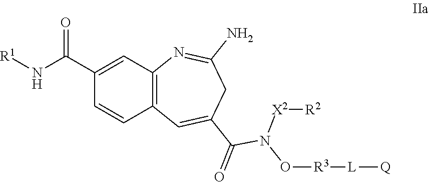

An exemplary embodiment of the 2-amino-4-carboxamide-benzazepine-linker compound is selected from Table 2. Each compound was characterized by mass spectrometry and shown to have the mass indicated.

TABLE 2a
2-amino-4-carboxamide-benzazepine-linker (2Am4CBza-L) Formula II compounds

| 2Am4CBza-L | Structure | MW |
|---|---|---|
| 2Am4CBza-L-1 | 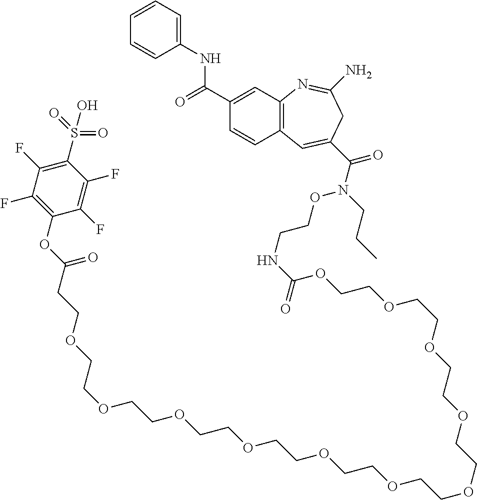 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)Nc3ccccc3)cc2N=C(N)C1] | 1206.2 |

TABLE 2b
2-amino-4-carboxamide-benzazepine-linker (2Am4CBza-L) Formula II compounds

| 2Am4CBza-L | Structure | MW |
|---|---|---|
| 2Am4CBza-L-2 | 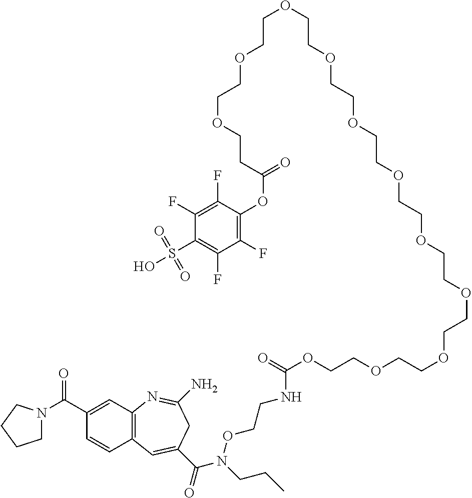 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCCC3)cc2N=C(N)C1] | 1184.2 |
| 2Am4CBza-L-3 | 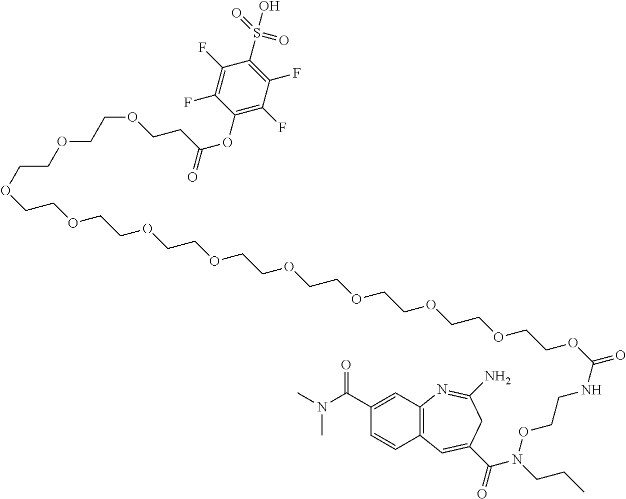 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N(C)C)cc2N=C(N)C1] | 1158.2 |
| 2Am4CBza-L-4 | 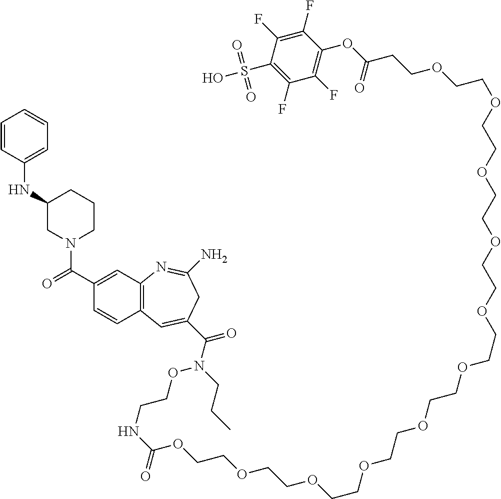 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCC[C@H](Nc4ccccc4)C3)cc2N=C(N)C1] | 1289.3 |
| 2Am4CBza-L-5 | 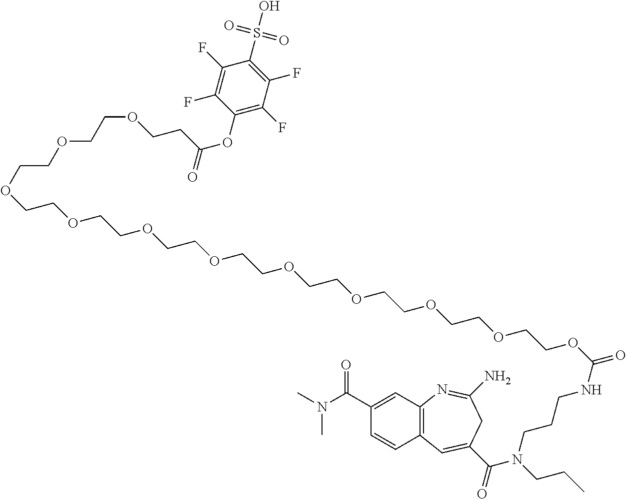 [SMILES: CCCN(CCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N(C)C)cc2N=C(N)C1] | 1156.2 |
| 2Am4CBza-L-6 | 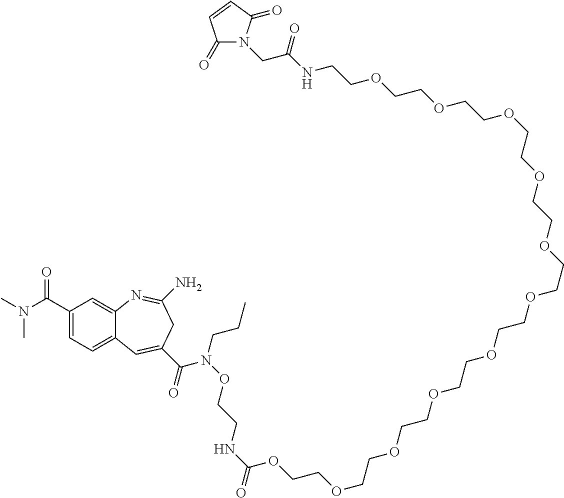 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1] | 1038.2 |
| 2Am4CBza-L-7 | 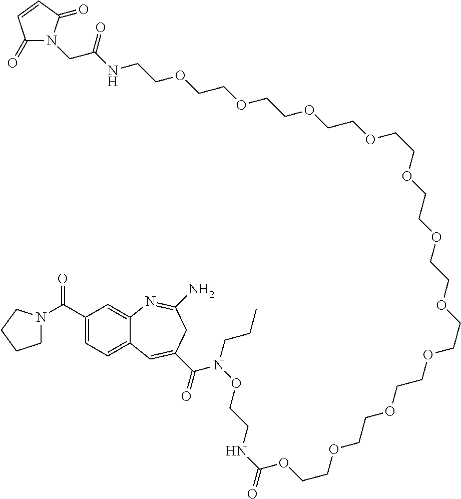 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCCC3)cc2N=C(N)C1] | 1064.2 |
| 2Am4CBza-L-8 | 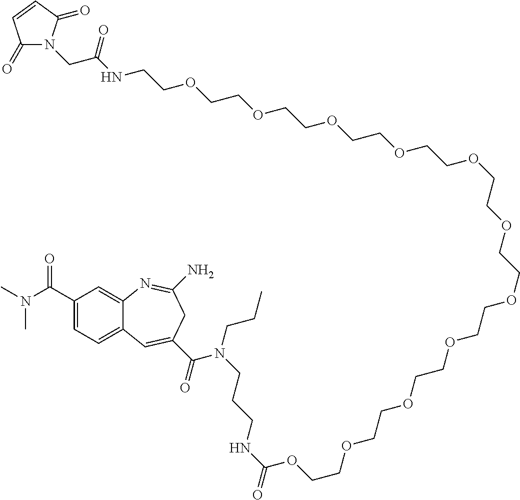 [SMILES: CCCN(CCCNC(=O)OCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1] | 1036.2 |
| 2Am4CBza-L-9 | 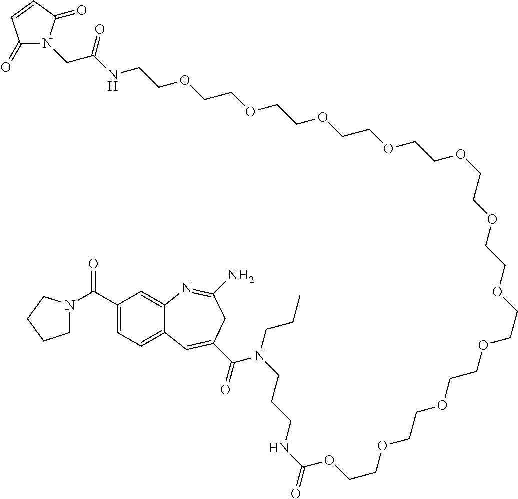 [SMILES: CCCN(CCCNC(=O)OCCOCCOCCOCCOCCOCCOCCOCCOCCOCCOCCNC(=O)CN1C(=O)C=CC1=O)C(=O)C1=Cc2ccc(C(=O)N3CCCC3)cc2N=C(N)C1] | 1062.2 |
| 2Am4CBza-L-10 | 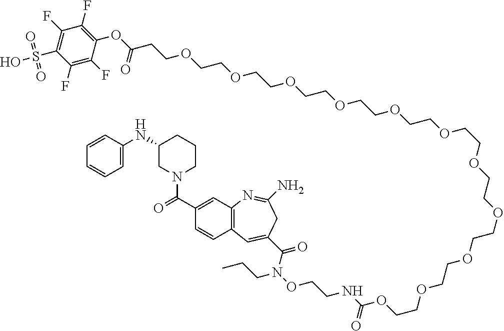 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCC[C@@H](Nc4ccccc4)C3)cc2N=C(N)C1] | 1289.3 |
| 2Am4CBza-L-11 | 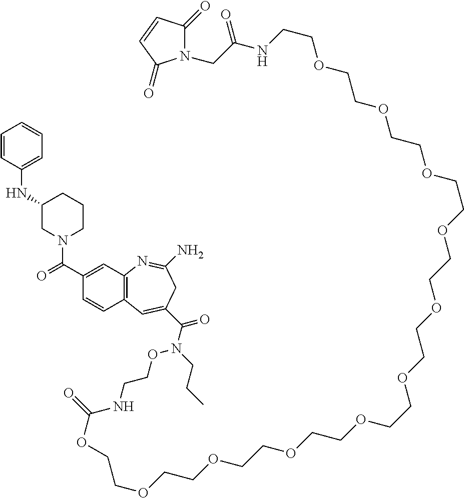 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCC[C@@H](Nc4ccccc4)C3)cc2N=C(N)C1] | 1169.3 |
| 2Am4CBza-L-12 | 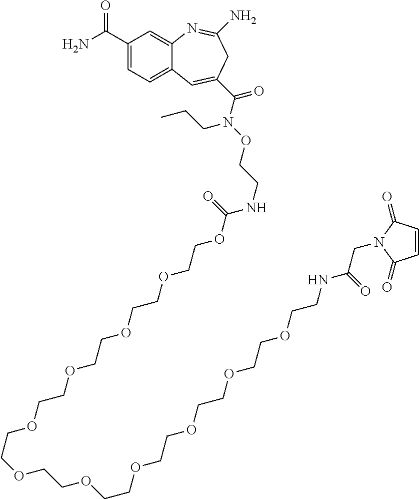 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(N)=O)cc2N=C(N)C1] | 1010.1 |
| 2Am4CBza-L-13 | 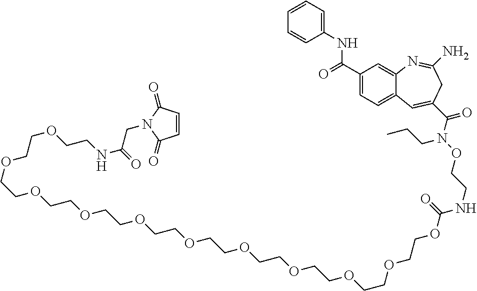 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)Nc3ccccc3)cc2N=C(N)C1] | 1086.2 |
| 2Am4CBza-L-14 | 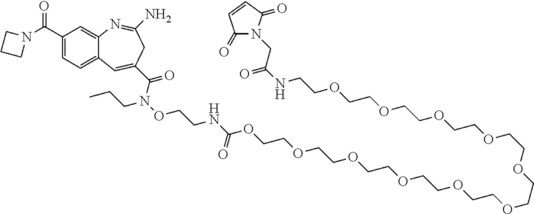 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCC3)cc2N=C(N)C1] | 1050.2 |
| 2Am4CBza-L-15 | 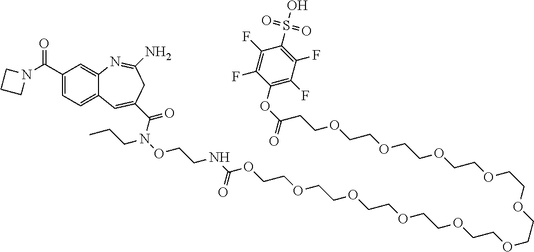 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCC3)cc2N=C(N)C1] | 1170.2 |
| 2Am4CBza-L-16 | 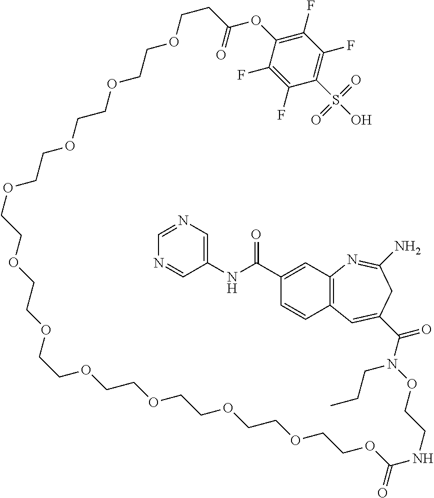 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)Nc3cncnc3)cc2N=C(N)C1] | 1208.2 |
| 2Am4CBza-L-17 | 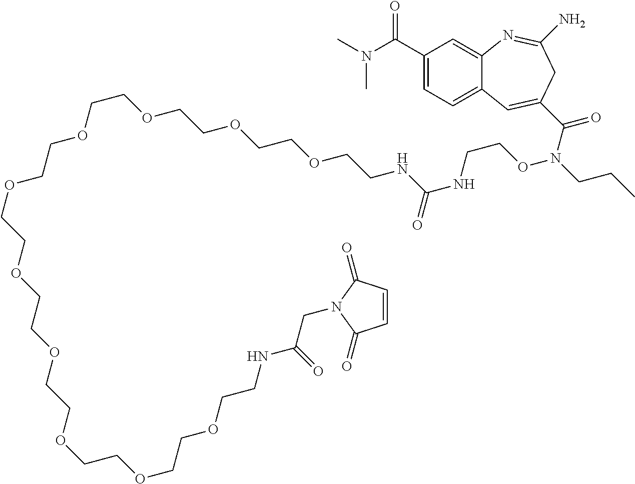 [SMILES: CCCN(OCCNC(=O)NCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1] | 1037.2 |
| 2Am4CBza-L-18 | 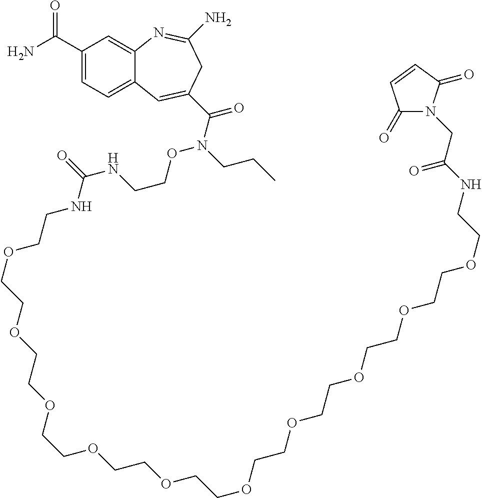 [SMILES: CCCN(OCCNC(=O)NCCOCCOCCOCCOCCOCCOCCOCCOCCOCCOCCNC(=O)CN1C(=O)C=CC1=O)C(=O)C1=Cc2ccc(C(N)=O)cc2N=C(N)C1] | 1009.1 |
| 2Am4CBza-L-19 | 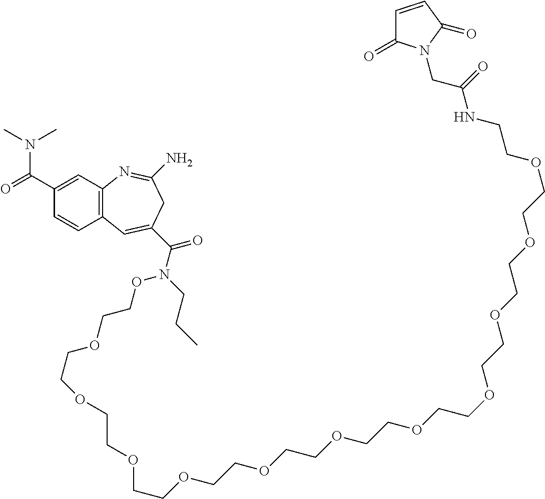 [SMILES: CCCN(OCCOCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1] | 995.1 |

### Immunoconjugates

Exemplary embodiments of immunoconjugates comprise an antibody covalently attached to one or more 2-amino-4-carboxamide-benzazepine (2Am4CBza) moieties by a linker, and having Formula L:

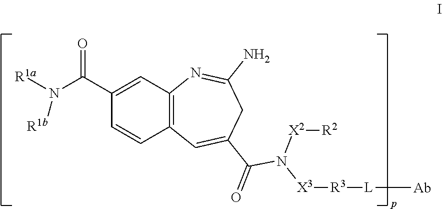

or a pharmaceutically acceptable salt thereof,
wherein:
Ab is the antibody wherein the antibody binds to a target selected from PD-L1, HER2, CEA; and TROP2;
p is an integer from 1 to 8;
X² and X³ are independently selected from the group consisting of a bond, C(═O), C(═O)N(R⁵), O, N(R⁵), S, S(O)₂, and S(O)₂N(R⁵);
R^{1a}, R^{1b}, and R² are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl; or R^{1a} and R^{1b} form a five- or six-membered heterocyclyl ring;
R³ is selected from the group consisting of:
—(C₁-C₁₂ alkyldiyl)-N(R⁵)—*;
—(C₁-C₁₂ alkyldiyl)-N(R⁵)—C(═O)*;
—(C₁-C₁₂ alkyldiyl)-N(R⁵)—C(═O)O—(C₃-C₁₂ carbocyclyldiyl)-*;
—(C₁-C₁₂ alkyldiyl)-N(R⁵)—(C₁-C₂₀ heteroaryldiyl)-*;
—(C₁-C₁₂ alkyldiyl)-N(R⁵)—(C₁-C₂₀ heteroaryldiyl)-(C₁-C₁₂ alkyldiyl)-*;
—(C₁-C₁₂ alkyldiyl)-N(R⁵)—S(O₂)—*;
—(C₁-C₁₂ alkyldiyl)-OC(═O)—(C₂-C₉ heterocyclyldiyl)-*;
—(C₁-C₁₂ alkyldiyl)-O—*;
—(C₁-C₁₂ alkyldiyl)-(C₃-C₁₂ carbocyclyldiyl)-*;
—(C₁-C₁₂ alkyldiyl)-(C₆-C₂₀ aryldiyl)-*;
—(C₁-C₁₂ alkyldiyl)-(C₆-C₂₀ aryl)-(C₁-C₁₂ alkyldiyl)-N(R⁵)—*;
—(C₁-C₁₂ alkyldiyl)-(C₂-C₉ heterocyclyldiyl)-(C₁-C₁₂ alkyldiyl)-N(R⁵)—*;
—(C₁-C₁₂ alkyldiyl)-(C₁-C₂₀ heteroaryldiyl)-N(R⁵)—*;
—(C₁-C₁₂ alkyldiyl)-(C₁-C₂₀ heteroaryldiyl)-*;
—(C₁-C₁₂ alkyldiyl)-(C₁-C₂₀ heteroaryldiyl)-(C₁-C₁₂ alkyldiyl)-*;
—(C₁-C₁₂ alkyldiyl)-(C₁-C₂₀ heteroaryldiyl)-(C₁-C₁₂ alkyldiyl)-N(R⁵)—*;
—(C₃-C₁₂ carbocyclyldiyl)-*;
—(C₃-C₁₂ carbocyclyldiyl)-(C₁-C₁₂ alkyldiyl)-N(R⁵)—*;
—(C₃-C₁₂ carbocyclyldiyl)-(C₁-C₁₂ alkyldiyl)-N(R⁵)—*;
—(C₃-C₁₂ carbocyclyldiyl)-NR⁵—C(═NR^{5a})—N(R⁵)—*;
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)—C(═NR^{5a})—N(R⁵)—*;
—(C₂-C₂₀ heterocyclyldiyl)-*;
—(C₂-C₉ heterocyclyldiyl)-(C₁-C₁₂ alkyldiyl)-N(R⁵)—*;
—(C₂-C₉ heterocyclyldiyl)-N(R⁵)—C(═NR^{5a})—N(R⁵)—*;
—(C₁-C₂₀ heteroaryldiyl)-*;
—(C₁-C₂₀ heteroaryldiyl)-(C₁-C₁₂ alkyldiyl)-N(R⁵)—*;
—(C₁-C₂₀ heteroaryldiyl)-(C₁-C₂ alkyldiyl)-O—*; and
—(C₁-C₂₀ heteroaryldiyl)-N(R⁵)—C(═NR^{5a})—N(R⁵)—*;
where the asterisk * indicates the attachment site of the linker L;
or R² and R³ together form a 5- or 6-membered heterocyclyl ring;
R⁵ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
L is selected from the group consisting of:
   —C(═O)-PEG-;
   —C(═O)-PEG-C(═O)N(R⁶)—(C₁-C₂ alkyldiyl)-C(═O)-Gluc-;
   —C(═O)-PEG-O—;
   —C(═O)-PEG-O—C(═O)—;
   —C(═O)-PEG-C(═O)—;
   —C(═O)-PEG-C(═O)-PEP-;
   —C(═O)-PEG-N(R⁶)—;
   —C(═O)-PEG-N(R⁶)—C(═O)—;
   —C(═O)-PEG-N(R⁶)-PEG-C(═O)-PEP-;
   —C(═O)-PEG-N(R⁶)₂-PEG-C(═O)-PEP-;
   —C(═O)-PEG-C(═O)-PEP-N(R⁶)—(C₁-C₁₂ alkyldiyl)-;
   —C(═O)-PEG-C(═O)-PEP-N(R⁶)—(C₁-C₁₂ alkyldiyl)N(R⁶)C(═O)—(C₂-C₅ monoheterocyclyldiyl)-;
   —C(═O)-PEG-SS-(C₁-C₁₂ alkyldiyl)-OC(═O)—;
   —C(═O)-PEG-SS-(C₁-C₁₂ alkyldiyl)-C(═O)—;
   —C(═O)—(C₁-C₂ alkyldiyl)-C(═O)-PEP-;
   —C(═O)—(C₁-C₂ alkyldiyl)-C(═O)-PEP-N(R⁶)—(C₁-C₁₂ alkyldiyl)-;
   —C(═O)—(C₁-C₁₂ alkyldiyl)-C(═O)-PEP-N(R⁶)—(C₁-C₁₂ alkyldiyl)-N(R⁵)—C(═O);
   —C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
   -succinimidyl-(CH₂)ₘ—C(═O)N(R⁶)-PEG-;
   -succinimidyl-(CH₂)ₘ—C(═O)N(R⁶)-PEG-C(═O)N(R⁶)—(C₁-C₁₂ alkyldiyl)-C(═O)-Gluc-;
   -succinimidyl-(CH₂)ₘ—C(═O)N(R⁶)-PEG-O—;
   -succinimidyl-(CH₂)ₘ—C(═O)N(R⁶)-PEG-O—C(═O)—;
   -succinimidyl-(CH₂)ₘ—C(═O)N(R⁶)-PEG-C(═O)—;
   -succinimidyl-(CH₂)ₘ—C(═O)N(R⁶)-PEG-N(R⁵)—;
   -succinimidyl-(CH₂)ₘ—C(═O)N(R⁶)-PEG-N(R⁵)—C(═O)—;
   -succinimidyl-(CH₂)ₘ—C(═O)N(R⁶)-PEG-C(═O)-PEP-;
   -succinimidyl-(CH₂)ₘ—C(═O)N(R⁶)-PEG-SS-(C₁-C₁₂ alkyldiyl)-OC(═O)—;
   -succinimidyl-(CH₂)ₘ—C(═O)-PEP-N(R⁶)—(C₁-C₁₂ alkyldiyl)-;
   -succinimidyl-(CH₂)ₘ—C(═O)-PEP-N(R⁶)—(C₁-C₁₂ alkyldiyl)N(R⁶)C(═O)—; and
   -succinimidyl-(CH₂)ₘ—C(═O)-PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ(CH₂)ₘ—; m is an integer from 1 to 5, and n is an integer from 2 to 50;
Gluc has the formula:

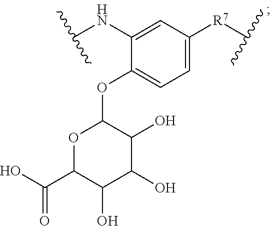

PEP has the formula:

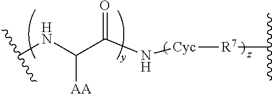

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl , NO₂, —OH, —OCH₃, and a glucuronic acid having the structure:

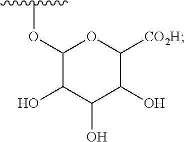 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —C_{H}═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH(OH)CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘ—CO₂H, —O(CH₂CH₂O),H, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the immunoconjugate of Formula I includes wherein the antibody is an antibody construct that has an antigen binding domain that binds PD-L1.
An exemplary embodiment of the immunoconjugate of Formula I includes wherein the antibody is selected from the group consisting of atezolizumab, durvalumab, and avelumab, or a biosimilar or a biobetter thereof.

An exemplary embodiment of the immunoconjugate of Formula I includes wherein the antibody is an antibody construct that has an antigen binding domain that binds HER2.

An exemplary embodiment of the immunoconjugate of Formula I includes wherein the antibody is selected from the group consisting of trastuzumab and pertuzumab, or a biosimilar or a biobetter thereof.

An exemplary embodiment of the immunoconjugate of Formula I includes wherein the antibody is an antibody construct that has an antigen binding domain that binds CEA.

An exemplary embodiment of the immunoconjugate of Formula I includes wherein the antibody is labetuzumab, or a biosimilar or a biobetter thereof.

An exemplary embodiment of the immunoconjugate of Formula I includes wherein the antibody is an antibody construct that has an antigen binding domain that binds TROP2.

An exemplary embodiment of the immunoconjugate of Formula I includes wherein the Trop2 antibody is a monoclonal antibody.

An exemplary embodiment of the immunoconjugate of Formula I includes wherein R^{1a} and R^{1b} are independently selected from a group consisting of optionally substituted C₆-C₂₀ aryl, C₂-C₉ heterocyclyl, and C₁-C₂₀ heteroaryl.

An exemplary embodiment of the immunoconjugate of Formula I includes wherein R^{1a} is optionally substituted C₆-C₂₀ aryl and R^{1b} is H.

An exemplary embodiment of the immunoconjugate of Formula I includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

An exemplary embodiment of the immunoconjugate of Formula I includes wherein X² is a bond, and R² is C₁-C₁₂ alkyl.

An exemplary embodiment of the immunoconjugate of Formula I includes wherein X³ is O and R³ is —(C₁-C₁₂ alkyldiyl)-N(R⁵)—*.

An exemplary embodiment of the immunoconjugate of Formula I includes wherein R³ is —CH₂CH₂CH₂NH—.

An exemplary embodiment of the immunoconjugate of Formula I includes wherein L is —C(═O)-PEG-C(═O)—.

An exemplary embodiment of the immunoconjugate of Formula I includes wherein L comprises PEG, and where n is 10 and m is 1.

An exemplary embodiment of the immunoconjugate of Formula I includes wherein AA₁ and AA₂ are independently selected from a side chain of a naturally-occurring amino acid.

An exemplary embodiment of the immunoconjugate of Formula I includes wherein AA₁ or AA₂ with an adjacent nitrogen atom form a 5-membered ring proline amino acid.

An exemplary embodiment of the immunoconjugate of Formula I includes wherein AA₁ and AA₂ are independently selected from H, —CH₃, —CH(CH₃)₂, —CH₂(C₆H5), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

An exemplary embodiment of the immunoconjugate of Formula I includes wherein AA₁ is —CH(CH₃)₂, and AA₂ is —CH₂CH₂CH₂NHC(O)NH₂.

An exemplary embodiment of the immunoconjugate has Formula Ia:

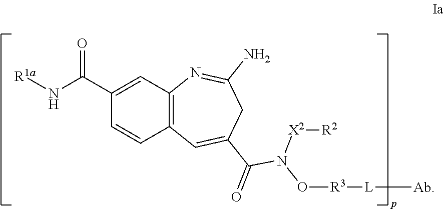

An exemplary embodiment of the immunoconjugate of Formula Ia includes wherein R^{1a} is a group selected from optionally substituted C₆-C₂₀ aryl, C₂-C₉ heterocyclyl, and C₁-C₂₀ heteroaryl.

An exemplary embodiment of the immunoconjugate of Formula Ia includes wherein R^{1a} is pyrimidinyl or pyridyl.

An exemplary embodiment of the immunoconjugate of Formula Ia includes wherein X² is a bond, and R² is C₁-C₁₂ alkyl.

An exemplary embodiment of the immunoconjugate of Formula Ia includes wherein R³ is —(C₁-C₁₂ alkyldiyl)-N(R⁵)—*.

An exemplary embodiment of the immunoconjugate of Formula Ia includes wherein X³—R³-L is selected from the group consisting of:

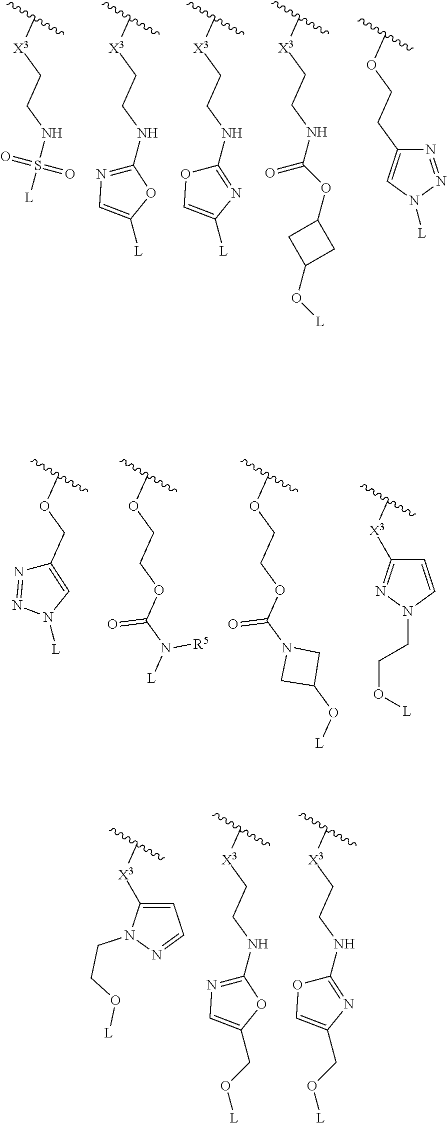

where the wavy line indicates the point of attachment to N.

The invention includes all reasonable and operable combinations, and permutations of the features, of the Formula I embodiments.

In certain embodiments, the immunoconjugate compounds of the invention include those with immunostimulatory activity. The antibody-drug conjugates of the invention selectively deliver an effective dose of an 2-amino-4-carboxamide-benzazepine drug to tumor tissue, whereby greater selectivity (i.e., a lower efficacious dose) may be achieved while increasing the therapeutic index (“therapeutic window”) relative to unconjugated 2-amino-4-carboxamide-benzazepine.

Drug loading is represented by p, the number of 2Am4CBza moieties per antibody in an immunoconjugate of Formula I. Drug (2Am4CBza) loading may range from 1 to about 8 drug moieties (D) per antibody. Immunoconjugates of Formula I include mixtures or collections of antibodies conjugated with a range of drug moieties, from 1 to about 8. In some embodiments, the number of drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p=n=1, 2, 3, 4, 5, 6, 7, or 8, or some range there between). Exemplary antibody-drug conjugates of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intrachain disulfide bonds, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

For some immunoconjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with a 2Am4CBza-linker compound of Formula II. In certain embodiments, higher drug loading, e.g. p>5, may cause aggregation, insolubility, toxicity, or loss of cellular permeability of certain antibody-drug conjugates. In certain embodiments, the average drug loading for an immunoconjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an immunoconjugate may be controlled in different ways, and for example, by: (i) limiting the molar excess of the 2Am4CBza-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of antibody-drug conjugate compounds with a distribution of one or more drug moieties attached to an antibody. The average number of drugs per antibody may be calculated from the mixture by a dual ELISA antibody assay, which is specific for antibody and specific for the drug. Individual immunoconjugate molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous immunoconjugate with a single loading value may be isolated from the conjugation mixture by electrophoresis or chromatography.

An exemplary embodiment of the immunoconjugate of Formula I is selected from the Tables 3a and 3b Immunoconjugates. In a co-culture of cancer cells with a cDC-enriched primary cell isolate, certain immunoconjugates of Tables 3a and 3b induce secretion of cytokine IL-12p70 which is relevant to mounting an immune response to cancer. Immunoconjugates targeting Clostridium difficile toxin B with bezlotox (bezlotoxumab), IC-28 and IC-33, were studied as isotype, non-tumor binding controls. Assessment of Immunoconjugate Activity In Vitro was conducted according to the methods of Example 203.

TABLE 3a
Immunoconjugates (IC)

| | | | | cDC |
|---|---|---|---|---|
| | | | | Activation |
| | | | | (IL-12p70 |
| | 2 Am4CBza - | | | Secretion) - |
| | linker | Ab | | EC₅₀ |
| IC No. | Table 2a | Antigen | DAR | (nM) |
| IC-1 | 2Am4CBzaL-1 | CEA.9-GlfhL2 | 2.7 | |
| | | CEA | | |
| IC-2 | 2Am4CBzaL-1 | trastuzumab | 2.5 | 1.3 |
| | | HER2 | | |

TABLE 3b
Immunoconjugates (IC)

| | | | | cDC |
|---|---|---|---|---|
| | | | | Activation |
| | | | | (IL-12p70 |
| | 2Am4CBza - | | | Secretion) - |
| | linker | Ab | | EC₅₀ |
| IC No. | Tables 2a-b | Antigen | DAR | (nM) |
| IC-3 | 2Am4CBzaL-1 | avelumab | 1.8 | |
| | | PD-L1 | | |
| IC-4 | 2Am4CBzaL-1 | PDL1.107-G1f | 2.0 | |
| | | PD-L1 | | |
| IC-5 | 2Am4CBzaL-1 | PDL1.53-IgG1f | 1.8 | |
| | | PD-L1 | | |
| IC-6 | 2Am4CBzaL-1 | PDL1.96-G1f | 2.1 | |
| | | PD-L1 | | |
| IC-7 | 2Am4CBzaL-1 | PDL1.110-G1f | 2.0 | |
| | | PD-L1 | | |
| IC-8 | 2Am4CBzaL-1 | PDL1.116-G1f | 2.1 | |
| | | PD-L1 | | |
| IC-9 | 2Am4CBzaL-1 | trastuzumab | 2.4 | 1.5 |
| | | HER2 | | |
| IC-10 | 2Am4CBzaL-5 | trastuzumab | 2.4 | |
| | | HER2 | | |
| IC-11 | 2Am4CBzaL-4 | trastuzumab | 2.6 | |
| | | HER2 | | |
| IC-12 | 2Am4CBzaL-7 | trastuzumab | 4.1 | 1.2 |
| | | HER2 | | |
| IC-13 | 2Am4CBzaL-8 | trastuzumab | 4.2 | |
| | | HER2 | | |
| IC-14 | 2Am4CBzaL-9 | trastuzumab | 4.1 | |
| | | HER2 | | |
| IC-15 | 2Am4CBzaL-6 | trastuzumab | 4.2 | 1.8 |
| | | HER2 | | |
| IC-16 | 2Am4CBzaL-3 | trastuzumab | 2.6 | 2.0 |
| | | HER2 | | |
| IC-17 | 2Am4CBzaL-10 | trastuzumab | 2.2 | |
| | | HER2 | | |
| IC-18 | 2Am4CBzaL-11 | trastuzumab | 3.9 | 5.1 |
| | | HER2 | | |
| IC-19 | 2Am4CBzaL-12 | trastuzumab | 3.8-4.1 | 1.5 |
| | | HER2 | | |
| IC-20 | 2Am4CBzaL-14 | trastuzumab | 3.2 | |
| | | HER2 | | |
| IC-21 | 2Am4CBzaL-6 | TROP2.1-G1f | 3.2, 4.1 | 0.9 |
| | | TROP2 | | |
| IC-22 | 2Am4CBzaL-12 | TROP2.1-G1f | 3.3 | |
| | | TROP2 | | |
| IC-23 | 2Am4CBzaL-1 | TROP2.1-G1f | 2.3 | |
| | | TROP2 | | |
| IC-24 | 2Am4CBzaL-15 | TROP2.1-G1f | 2.4 | |
| | | TROP2 | | |
| IC-25 | 2Am4CBzaL-16 | TROP2.1-G1f | 2.4 | |
| | | TROP2 | | |
| IC-26 | 2Am4CBzaL-18 | TROP2.1-G1f | 4.1 | |
| | | TROP2 | | |
| IC-27 | 2Am4CBzaL-17 | TROP2.1-G1f | 3.4, 4.2 | 1.8 |
| | | TROP2 | | |
| IC-28 | 2Am4CBzaL-6 | bezlotox-G1f | 4.5 | |
| | | C. difficile | | |
| IC-29 | 2Am4CBzaL-6 | TROP2.3-G1f | 4.1 | 0.7 |
| | | TROP2 | | |
| IC-30 | 2Am4CBzaL-6 | TROP2.5-G1f | 3.3 | 1.1 |
| | | TROP2 | | |
| IC-31 | 2Am4CBzaL-19 | TROP2.5-G1f | 3.2 | |
| | | TROP2 | | |
| IC-32 | 2Am4CBzaL-13 | PDL1.110-G1f | 3.1 | 0.6 |
| | | PD-L1 | | |
| IC-33 | 2Am4CBzaL-13 | bezlotox-G1f | 3.9 | |
| | | C. difficile | | |

### Pharmaceutical Compositions of Immunoconjugates

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of adjuvants linked to the same positions on the antibody construct and/or immunoconjugates that have the same number of 2Am4CBza adjuvants linked to different positions on the antibody construct, that have different numbers of adjuvants linked to the same positions on the antibody construct, or that have different numbers of adjuvants linked to different positions on the antibody construct.

In an exemplary embodiment, a composition comprising the immunoconjugate compounds comprises a mixture of the immunoconjugate compounds, wherein the average drug (2Am4CBza) loading per antibody in the mixture of immunoconjugate compounds is about 2 to about 5.

A composition of immunoconjugates of the invention can have an average adjuvant to antibody construct ratio of about 0.4 to about 10. A skilled artisan will recognize that the number of 2Am4CBza adjuvants conjugated to the antibody construct may vary from immunoconjugate to immunoconjugate in a composition comprising multiple immunoconjugates of the invention, and, thus, the adjuvant to antibody construct (e.g., antibody) ratio can be measured as an average, which may be referred to as the drug to antibody ratio (DAR). The adjuvant to antibody construct (e.g., antibody) ratio can be assessed by any suitable means, many of which are known in the art.

The average number of adjuvant moieties per antibody (DAR) in preparations of immunoconjugates from conjugation reactions may be characterized by conventional means such as mass spectrometry, ELISA assay, and HPLC. The quantitative distribution of immunoconjugates in a composition in terms of p may also be determined. In some instances, separation, purification, and characterization of homogeneous immunoconjugates where p is a certain value from immunoconjugates with other drug loadings may be achieved by means such as reverse phase HPLC or electrophoresis.

In some embodiments, the composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.10% (w/w) to about 10% (w/w).

Method of Treating Cancer with Immunoconjugates

The invention provides a method for treating cancer. The method includes administering a therapeutically effective amount of an immunoconjugate as described herein (e.g., as a composition as described herein) to a subject in need thereof, e.g., a subject that has cancer and is in need of treatment for the cancer. The method includes administering a therapeutically effective amount of an immunoconjugate (IC) selected from Table 3.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding PD-L1 (e.g., atezolizumab, durvalumab, avelumab, biosimilars thereof, or biobetters thereof). In some embodiments, methods for treating breast cancer include administering an immunoconjugate containing an antibody construct that is capable of binding PD-L1 (e.g., atezolizumab, durvalumab, avelumab, biosimilars thereof, or biobetters thereof). In some embodiments, methods for treating triple-negative breast cancer include administering an immunoconjugate containing an antibody construct that is capable of binding PD-L1 (e.g., atezolizumab, durvalumab, avelumab, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias.

Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Atezolizumab, durvalumab, avelumab, biosimilars thereof, and biobetters thereof are known to be useful in the treatment of cancer, particularly breast cancer, especially triple negative (test negative for estrogen receptors, progesterone receptors, and excess HER2 protein) breast cancer, bladder cancer, and Merkel cell carcinoma. The immunoconjugate described herein can be used to treat the same types of cancers as atezolizumab, durvalumab, avelumab, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

The immunoconjugate is administered to a subject in need thereof in any therapeutically effective amount using any suitable dosing regimen, such as the dosing regimens utilized for atezolizumab, durvalumab, avelumab, biosimilars thereof, and biobetters thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg to the subject.

The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding HER2 (e.g. trastuzumab, pertuzumab, biosimilars, or biobetters thereof) and PD-L1 (e.g., atezolizumab, durvalumab, avelumab, biosimilars, or biobetters thereof). In some embodiments, methods for treating colon cancer lung cancer, renal cancer, pancreatic cancer, gastric cancer, and esophageal cancer include administering an immunoconjugate containing an antibody construct that is capable of binding CEA, or tumors over-expressing CEA (e.g. labetuzumab, biosimilars, or biobetters thereof).

In some embodiments, the cancer is susceptible to a pro-inflammatory response induced by TLR7 and/or TLR8.

### EXAMPLES

### Preparation of 2-amino-4-carboxamide-benzazepine compounds (2Am4CBza) and intermediates

### Example 1 Synthesis of 4-[3-[2-[2-[2-[2-[2-[2-[2-[2-[2-[2-[2-[[2-amino-8-(phenylcarbamoyl)-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoyloxy]-2,3,5,6-tetrafluoro-benzenesulfonic acid, 2Am4CBza-L-1

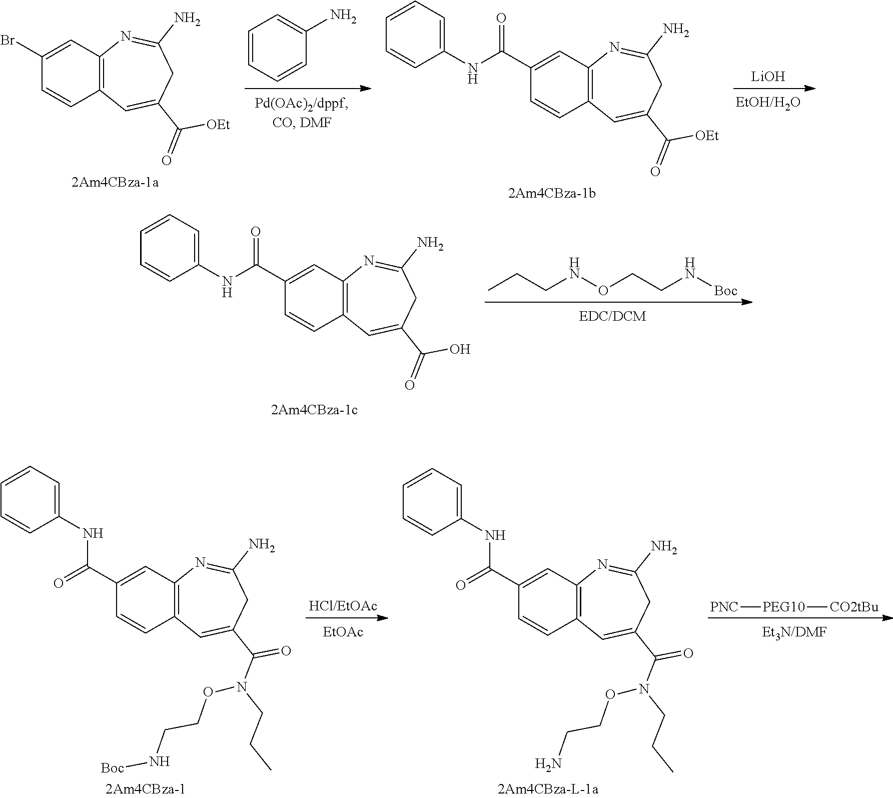

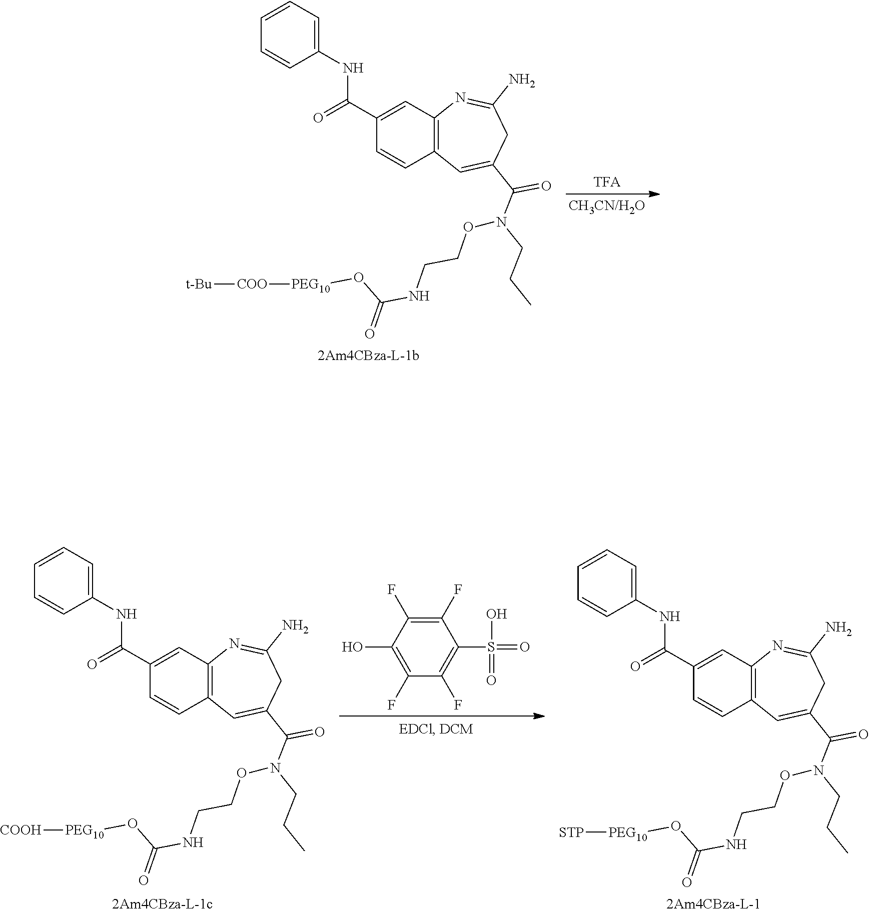

### Preparation of ethyl 2-amino-8-(phenylcarbamoyl)-3H-benzo[b]azepine-4-carboxylate, 2Am4CBza-1b

To a solution of ethyl 2-amino-8-bromo-3H-1-benzazepine-4-carboxylate, 2Am4CBza-1a (2 g, 6.47 mmol (millimoles), 1 eq) and aniline (3.01 g, 32.3 mmol, 2.95 mL, 5 eq) in DMF (20 mL) was added Pd(OAc)₂ (218 mg, 970 umol (micromoles), 0.15 eq), DPPF (610 mg, 1.10 mmol, 0.17 eq) and TEA (3.27 g, 32.3 mmol, 4.50 mL, 5 eq) under N₂. The suspension was degassed under vacuum and purged with carbon monoxide gas, CO several times. The mixture was stirred under CO (50 psi) at 80° C. for 12 h. The reaction mixture was filtered and concentrated under reduced pressure. The residue was diluted with H₂O (60 mL) and extracted with EtOAc (50 mL×3). The combined organic layers were washed with brine (30 mL×3), dried over Na₂SO₄, filtered and concentrated under reduced pressure. The residue was purified by column chromatography (SiO₂, Petroleum ether/Ethyl acetate=1/0 to 0/1) and then (SiO₂, EtOAc/MeOH=1/0 to 10/1) to give 2Am4CBza-1b (0.82 g, 2.35 mmol, 36.28% yield) as a yellow solid.

### 2 Preparation of 2-amino-8-(phenylcarbamoyl)-3H-1-benzazepine-4-carboxylic acid, 2Am4CBza-1c

To a solution of 2Am4CBza-1b (810 mg, 2.32 mmol, 1 eq) in EtOH (8 mL) was added a solution of LiOH·H₂O (486.40 mg, 11.59 mmol, 5 eq) in H₂O (2 mL). The mixture was stirred at 20° C. for 3 h, and then adjusted to pH=6-7 with 1N HCl and concentrated in vacuum. The residue was purified by prep-HPLC (column: Phenomenex luna C18 250*50 mm*10 um; mobile phase: [water(0.05% HCl)-ACN]; B %: 5%-35%, 10 min) to give 2Am4CBza-1c (180 mg, 560.17 umol, 24.16% yield) as a white solid. ¹H NMR (DMSO-d₄, 400 MHz) δ12.43 (s, 1H), 10.49 (s, 1H), 10.09 (s, 1H), 9.07 (br s, 1H), 7.99 (d, J=8.4 Hz, 1H), 7.96-7.93 (m, 2H), 7.87-7.83 (m, 1H), 7.79 (d, J=7.6 Hz, 2H), 7.38 (t, J=7.6 Hz, 2H), 7.16-7.11 (m, 1H), 3.52 (s, 2H).

### Preparation of tert-butyl N-[2-[[2-amino-8-(phenylcarbamoyl)-3H-1-benzazepine-4-carbonyl]-propyl-amino]oxyethyl]carbamate, 2Am4CBza-1

To a mixture of 2Am4CBza-1c (100 mg, 311 umol, 1 eq) and tert-butyl N-[2-(propylaminooxy)ethyl]carbamate (88.3 mg, 405 umol, 1.3 eq) in DCM (3 mL) and DMA (1.5 mL) was added EDCI (238 mg, 1.24 mmol, 4 eq) at 25° C. under N₂, and then stirred at 25° C. for 1 hours. The mixture was concentrated in vacuum to remove DCM, the residue was diluted with H₂O (10 mL), then the pH of the mixture was adjusted to about 8 with aq NaHCO₃, extracted with ethyl acetate (20 mL*3). The combined organic phase was washed with brine (20 mL), dried with anhydrous Na₂SO₄, filtered and concentrated in vacuum. The residue was purified by prep-HPLC column: Phenomenex Luna 80*30 mm*3 um; mobile phase: [water(0.1% TFA)-ACN]; B %: 15%-45%, 8 min to afford 2Am4CBza-1 (80 mg, 153 umol, 49.28% yield) as white solid. ¹H NMR (MeOD, 400 MHz) δ8.03-7.93 (m, 2H), 7.77 (d, J=8.0 Hz, 1H), 7.74-7.67 (m, 2H), 7.52 (s, 1H), 7.39 (t, J=8.0 Hz, 2H), 7.24-7.14 (m, 1H), 3.95 (t, J=5.2 Hz, 2H), 3.76 (t, J=7.2 Hz, 2H), 3.43 (s, 2H), 3.27 (t, J=5.2 Hz, 2H), 1.78 (sxt, J=7.2 Hz, 2H), 1.37 (s, 9H), 1.00 (t, J=7.2 Hz, 3H).

### Preparation of 2-amino-N4-(2-aminoethoxy)-N8-phenyl-N4-propyl-3H-benzo [b]azepine-4,8-dicarboxamide, 2Am4CBza-L-1a

To a mixture of 2Am4CBza-1 (80 mg, 153 umol, 1 eq) in EtOAc (1 mL) was added HCl/EtOAc (4 M, 3 mL, 78.0 eq) in one portion at 25° C., and then stirred at 25° C. for 1 hour. The mixture was concentrated in vacuum to afford 2Am4CBza-L-1a (70 mg, 152.85 umol, 99.66% yield, HCl) as white solid.

### Preparation of tert-butyl 3-[2-[2-[2-[2-[2-[2-[2-[2-[2-[2-(4-nitrophenoxy)carbonyl oxyethoxy]ethoxy]ethoxy]ethoxy]ethoxy]ethoxy]ethoxy]ethoxy]ethoxy]ethoxy]propanoate, PNC-PEG10-CO2tBu

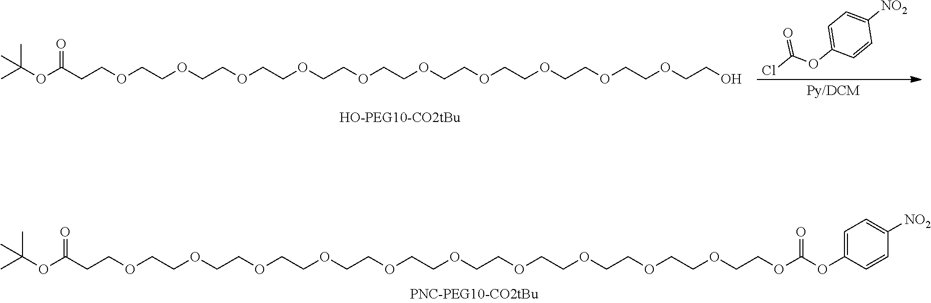 [SMILES: CC(C)(C)OC(=O)CCOCCOCCOCCOCCOCCOCCOCCOCCOCCOCCO ; O=C(Cl)Oc1ccc([N+](=O)[O-])cc1 ; CC(C)(C)OC(=O)CCOCCOCCOCCOCCOCCOCCOCCOCCOCCOCCOC(=O)Oc1ccc([N+](=O)[O-])cc1]

To a mixture of tert-butyl 3-[2-[2-[2-[2-[2-[2-[2-[2-[2-(2-hydroxyethoxy)ethoxy]ethoxy]ethoxy]ethoxy]ethoxy]ethoxy]ethoxy]ethoxy]ethoxy]propanoate, HO-PEG10-CO2tBu (1 g, 1.70 mmol, 1.0 eq) and (4-nitrophenyl)carbonochloridate (378 mg, 1.87 mmol, 1.1 eq) in DCM (20 mL) was added pyridine (202 mg, 2.56 mmol, 206 uL, 1.5 eq) at 0° C. The mixture was stirred at 25° C. for 2 hrs. The pH of the mixture was adjusted to about 4 with 1M HCl. The residue was poured into ice-water (w/w=1/1) (100 mL) and stirred for 10 min. The aqueous phase was extracted with DCM (50 mL×3). The combined organic phase was dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0:0/1, Ethyl acetate/Methanol=1/0:2/1) to afford PNC-PEG10-CO2tBu (650 mg, 865 umol, 50.73% yield) as colorless oil. ¹H NMR (MeOD, 400 MHz) δ 8.38-8.27 (m, 2H), 7.54-7.45 (m, 2H), 4.47-4.42 (m, 2H), 3.80-3.53 (m, 40H), 2.53-2.44 (m, 2H), 1.50-1.41 (m, 9H).

### Preparation of tert-butyl 3-[2-[2-[2-[2-[2-[2-[2-[2-[2-[2-[2-[[2-amino-8-(phenyl carbamoyl)-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoate, 2Am4CBza-L-1b

To a mixture of 2Am4CBza-L-1a (70 mg, 153 umol, 1 eq, HCl) and PNC-PEG10-CO2tBu (126 mg, 168 umol, 1.1 eq) in DMF (2.5 mL) was added Et₃N (38.7 mg, 382 umol, 2.5 eq) at 25° C. under N₂, and then stirred at 25° C. for 1 hours. The mixture was filtered and purified by prep-HPLC column: Phenomenex luna C18 100*40 mm*5 um; mobile phase: [water(0.1% TFA)-ACN]; B %: 15%-55%, 8 min. to give 2Am4CBza-L-1b (70 mg, 67.7 umol, 44.28% yield) as colorless oil.

### Preparation of 3-[2-[2-[2-[2-[2-[2-[2-[2-[2-[2-[2-[[2-amino-8-(phenylcarbamoyl)-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoic acid, 2Am4CBza-L-1c

To a mixture of 2Am4CBza-L-1b (70 mg, 67.7 umol, 1 eq) in CH₃CN (1 mL) and H₂O (1 mL) was added TFA (77.2 mg, 677 umol, 10 eq) at 25° C. under N₂, and then stirred at 80° C. for 2 hours. The pH of the mixture was adjusted to ˜5 with aq NaHCO₃ at 0° C., then extracted with DCM/i-PrOH=3/1(10 mL*3). The combined organic phase dried with anhydrous Na₂SO₄, filtered and concentrated in vacuum to give 2Am4CBza-L-1c (65 mg, 66.46 umol, 98.18% yield) as light yellow oil.

### Preparation of 2Am4CBza-L-1

To a mixture of 2Am4CBza-L-1c (60 mg, 61.34 umol, 1 eq) and sodium; 2,3,5,6-tetrafluoro-4-hydroxy-benzenesulfonate, STP (49.3 mg, 184 umol, 3 eq) in DCM (2 mL) and DMA (0.5 mL) was added EDCI (35.3 mg, 184 umol (micromoles), 3 eq) at 25° C. under N₂, and then stirred at 25° C. for 1 hours. The mixture was concentrated in vacuum, and then purified by prep-HPLC column: Phenomenex Luna 80*30 mm*3 um; mobile phase: [water(0.1% TFA)-ACN]; B %: 15%-40%, 8 min. to give 2Am4CBza-L-1 (40 mg, 33.16 umol, 54.06% yield) as white solid. ¹H NMR (MeOD, 400 MHz) δ8.06-7.95 (m, 2H), 7.73 (dd, J=5.0, 7.6 Hz, 3H), 7.49-7.43 (m, 1H), 7.45 (s, 1H), 7.38 (t, J=8.0 Hz, 2H), 7.23-7.13 (m, 1H), 3.98 (t, J=5.0 Hz, 2H), 3.85 (t, J=5.6 Hz, 2H), 3.80 (br d, J=4.4 Hz, 2H), 3.75 (t, J=7.2 Hz, 2H), 3.66-3.54 (m, 38H), 3.51-3.42 (m, 4H), 2.96 (t, J=6.0 Hz, 2H), 1.77 (sxt, J=7.2 Hz, 2H), 1.00 (t, J=7.2 Hz, 3H). HPLC: 99.47% (220 nm), 99.31% (254 nm). LC/MS [M+H] 1206.4 (calculated); LC/MS [M+H] 1206.7 (observed). LC/MS [M+H] 1206.4 (calculated); LC/MS [M+H] 1206.7 (observed).

Example 4 Synthesis of 4-[3-[2-[2-[2-[2-[2-[2-[2-[2-[2-[2-[2-[[2-amino-8-[(3S)-3-anilinopiperidine-1-carbonyl]-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoyloxy]-2,3,5,6-tetrafluoro-benzenesulfonic acid, 2Am4CBza-L-4

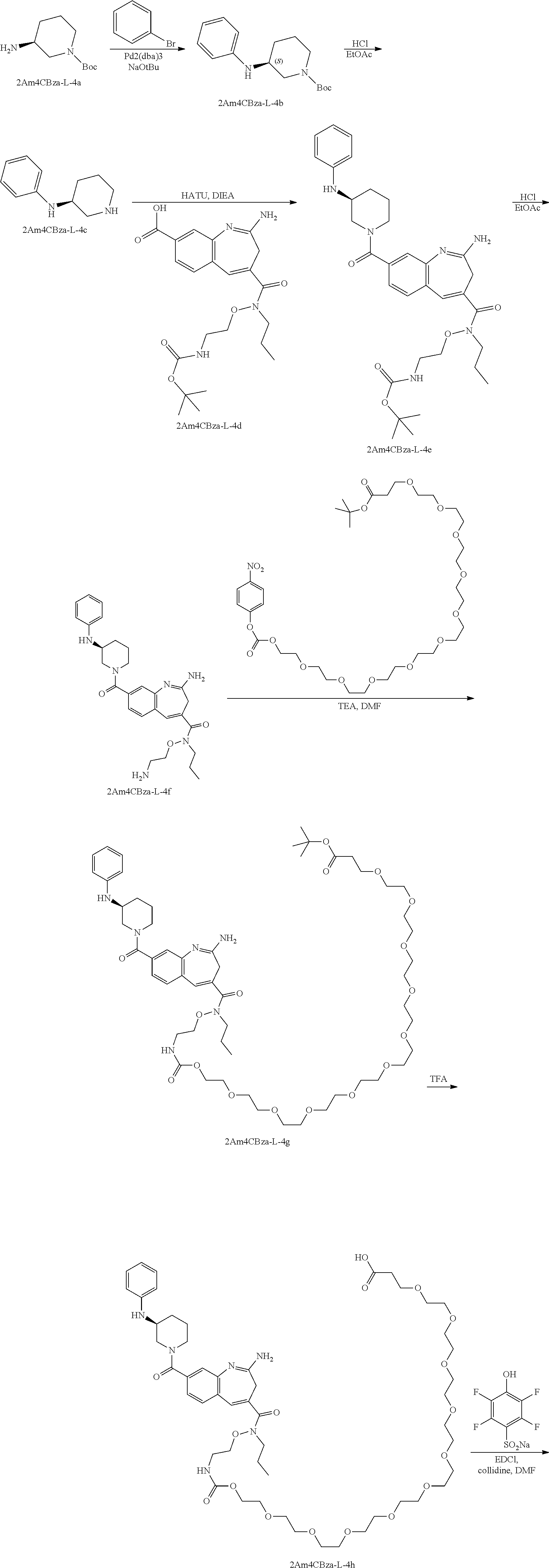

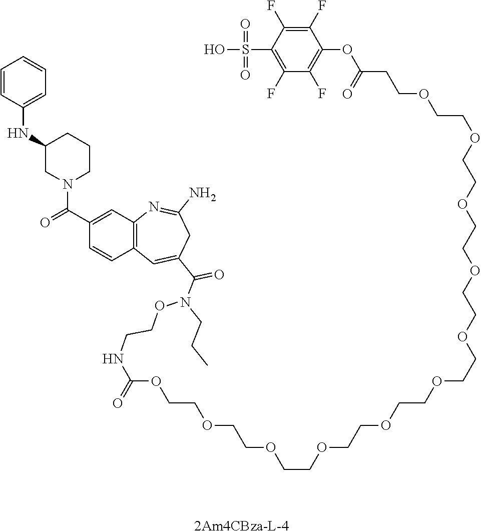 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCC[C@H](Nc4ccccc4)C3)cc2N=C(N)C1 ; "2Am4CBza-L-4"]

### Preparation of tert-butyl (3S)-3-anilinopiperidine-1-carboxylate, 2Am4CBza-L-4b

To a solution of tert-butyl (3S)-3-aminopiperidine-1-carboxylate, 2Am4CBza-L-4a (1.00 g, 4.99 mmol, 1.0 eq) and bromobenzene (780 mg, 4.99 mmol, 526 uL, 1.0 eq) in toluene (10.0 mL) was added sodium tert-butoxide, NaOtBu (580 mg, 5.99 mmol, 1.2 eq), 2-(2-dicyclohexylphosphanylphenyl)-N,N-dimethyl-aniline (140 mg, 349 umol, 0.07 eq) and Tris(dibenzylideneacetone)dipalladium(0), Pd₂(dba)₃, CAS Reg. No. 51364-51-3, (230 mg, 249 umol, 0.05 eq) at 20° C. under N₂. The mixture was stirred at 100° C. for 24 h. The mixture was diluted with water (30 mL) and extracted with EtOAc (30 mL×3). The organic layer was washed with brine (20 mL), dried over Na₂SO₄, filtered and concentrated. The residue was purified by flash silica gel chromatography (ISCO; 12 g SepaFlash Silica Flash Column, Eluent of 0˜50% Ethyl acetate/Petroleum ethergradient: 45 mL/min) to give 2Am4CBza-L-4b (1.2 g, 4.34 mmol, 86.9% yield) as light yellow solid. ¹H NMR (CDCl₃, 400 MHz) δ7.18 (dd, J=7.6, 8.4 Hz, 2H), 6.77-6.60 (m, 3H), 4.09-3.95 (m, 1H), 3.83-3.62 (m, 2H), 3.45-3.33 (m, 1H), 3.09 (br d, J=9.6 Hz, 1H), 2.98-2.78 (m, 1H), 2.04-1.94 (m, 1H), 1.80-1.71 (m, 1H), 1.65-1.53 (m, 2H), 1.46 (s, 9H). LC/MS [M+H] 277.2 (calculated); LC/MS [M+H] 277.2 (observed).

### Preparation of (3S)-N-phenylpiperidin-3-amine, 2Am4CBza-L-4c

To a solution of 2Am4CBza-L-4b (1.20 g, 4.34 mmol, 1.0 eq) in EtOAc (5.00 mL) was added HCl/EtOAc (4 M, 18.0 mL, 17.0 eq). The mixture was stirred at 20° C. for 1 h. The mixture was concentrated to give 2Am4CBza-L-4c (900 mg, 3.61 mmol, 83.1% yield, 2 HCl) as white solid. ¹H NMR (MeOD, 400 MHz) δ7.59-7.52 (m, 2H), 7.49-7.41 (m, 3H), 3.94 (tt, J=4.0, 10.8 Hz, 1H), 3.62-3.54 (m, 1H), 3.40 (d, J=12.0 Hz, 1H), 3.20 (t, J=11.6 Hz, 1H), 3.04 (dt, J=3.2, 12.4 Hz, 1H), 2.26-2.07 (m, 2H), 1.93-1.78 (m, 2H). LC/MS [M+H] 177.2 (calculated); LC/MS [M+H] 177.2 (observed).

### Preparation of 2Am4CBza-L-4e

To a solution of 2-amino-4-[2-(tert-butoxycarbonylamino)ethoxy-propyl-carbamoyl]-3H-1-benzazepine-8-carboxylic acid, 2Am4CBza-L-4d (250 mg, 559 umol, 1.0 eq) in DMF (1.00 mL) was added HATU (210 mg, 559 umol, 1.0 eq), DIEA (290 mg, 2.24 mmol, 390 uL, 4.0 eq) and (3S)-N-phenylpiperidin-3-amine, 2Am4CBza-L-4c (130 mg, 603 umol, 1.08 eq, HCl), and then stirred at 0° C. for 1 h. The mixture was filtered and purified by prep-HPLC (column: Phenomenex Luna 80*30 mm*3 um; mobile phase: [water(0.1% TFA)-ACN]; B %: 5%-50%, 8 min) to give 2Am4CBza-L-4e (160 mg, 222 umol, 39.7% yield, TFA) as white solid. ¹H NMR (MeOD, 400 MHz) δ7.76-7.38 (m, 3H), 7.36-7.15 (m, 2H), 7.11-6.90 (m, 2H), 6.63-6.42 (m, 2H), 3.95-3.92 (m, 2H), 3.89-3.61 (m, 4H), 3.60-3.32 (m, 4H), 3.28-3.23 (m, 2H), 3.08-2.96 (m, 1H), 2.20-1.94 (m, 2H), 1.84-1.67 (m, 4H), 1.38 (s, 9H), 0.99 (t, J=7.6 Hz, 3H). LC/MS [M+H] 605.3 (calculated); LC/MS [M+H] 605.3 (observed).

### Preparation of 2-amino-N-(2-aminoethoxy)-8-[(3 S)-3-anilinopiperidine-1-carbonyl]-N-propyl-3H-1-benzazepine-4-carboxamide, 2Am4CBza-L-4f

To a solution of 2Am4CBza-L-4e (90.0 mg, 125 umol, 1.0 eq, TFA) in EtOAc (5.00 mL) was added HCl/EtOAc (4 M, 10.0 mL, 319.0 eq). The mixture was stirred at 20° C. for 1 h, and then concentrated to give 2Am4CBza-L-4f (78.0 mg, crude, 2HCl) as white solid. LC/MS [M+H] 505.3 (calculated); LC/MS [M+H] 505.3 (observed).

### Preparation of tert-butyl 3-[2-[2-[2-[2-[2-[2-[2-[2-[2-[2-[2-[[2-amino-8-[(3S)-3-anilinopiperidine-1-carbonyl]-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoate, 2Am4CBza-L-4g

To a solution of 2Am4CBza-L-4f (70.0 mg, 121 umol, 1.0 eq, 2HCl) in DMF (1.00 mL) was added Et₃N (50.0 mg, 484 umol, 70.0 uL, 4.0 eq) and tert-butyl 3-[2-[2-[2-[2-[2-[2-[2-[2-[2-[2-(4-nitrophenoxy)carbonyloxyethoxy]ethoxy]ethoxy]ethoxy]ethoxy]ethoxy]ethoxy]ethoxy]ethoxy]e thoxy]propanoate (90.0 mg, 121 umol, 1.0 eq). The mixture was stirred at 0° C. for 1 h, and then diluted with water (10 mL) and extracted with EtOAc (20 mL×3). The organic layer was washed with brine (10 mL), dried over Na₂SO₄, filtered and concentrate to give 2Am4CBza-L-4g (200 mg, crude) as light yellow oil. LC/MS [M+H] 1117.6 (calculated); LC/MS [M+H]1117.6 (observed).

### Preparation of 3-[2-[2-[2-[2-[2-[2-[2-[2-[2-[2-[2-[[2-amino-8-[(3S)-3-anilinopiperidine-1-carbonyl]-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoic acid, 2Am4CBza-L-4h

To a solution of 2Am4CBza-L-4g (200 mg, 179 umol, 1.0 eq) in CH₃CN (1.00 mL) and H₂O (1.00 mL) was added TFA (200 mg, 1.79 mmol, 130 uL, 10.0 eq), and then stirred at 80° C. for 1 h. The mixture was concentrated to give a residue. The residue was purified by prep-HPLC (column: Phenomenex Luna 80*30 mm*3 um; mobile phase: [water(0.1% TFA)-ACN]; B %: 5%-40%, 8 min) to give 2Am4CBza-L-4h (50 mg, 42.5 umol, 23.7% yield, TFA) as colorless oil. LC/MS [M+H] 1061.5 (calculated); LC/MS [M+H] 1061.5 (observed).

### Preparation of 2Am4CBza-L-4

To a solution of 2Am4CBza-L-4h (40.0 mg, 34.0 umol, 1.0 eq, TFA) and 2,3,5,6-tetrafluoro-4-hydroxy-benzenesulfonic acid (33.5 mg, 136 umol, 4.0 eq) in DCM (2.00 mL) and DMA (0.20 mL) was added EDCI (30.0 mg, 136 umol, 4.0 eq), and then stirred at 20° C. for 1 h.

The mixture was concentrated to give a residue. The residue was purified by prep-HPLC (column: Phenomenex Luna 80*30 mm*3 um; mobile phase: [water(0.1% TFA)-ACN]; B %: 15%-50%, 8 min) to give 2Am4CBza-L-4 (21 mg, 14.9 umol, 43.9% yield, TFA) as white solid. ¹H NMR (MeOD, 400 MHz) δ7.67-7.24 (m, 5H), 7.17-6.96 (m, 2H), 6.71-6.41 (m, 2H), 3.99-3.94 (m, 2H), 3.90-3.80 (m, 6H), 3.77-3.71 (m, 4H), 3.65-3.58 (m, 38H), 3.54-3.47 (m, 4H), 2.97 (t, J=6.0 Hz, 2H), 2.22-1.95 (m, 2H), 1.85-1.68 (m, 4H), 1.03-0.97 (m, 3H). LC/MS [M+H] 1289.5 (calculated); LC/MS [M+H] 1289.5 (observed).

### Example 6 Synthesis of 1-(2,5-dioxo-2,5-dihydro-1H-pyrrol-1-yl)-2-oxo-6,9,12,15,18,21,24,27,30,33-decaoxa-3-azapentatriacontan-35-yl(2-((2-amino-8-(dimethylcarbamoyl)-N-propyl-3H-benzo[b]azepine-4-carboxamido)oxy)ethyl)carbamate, 2Am4CBza-L-6

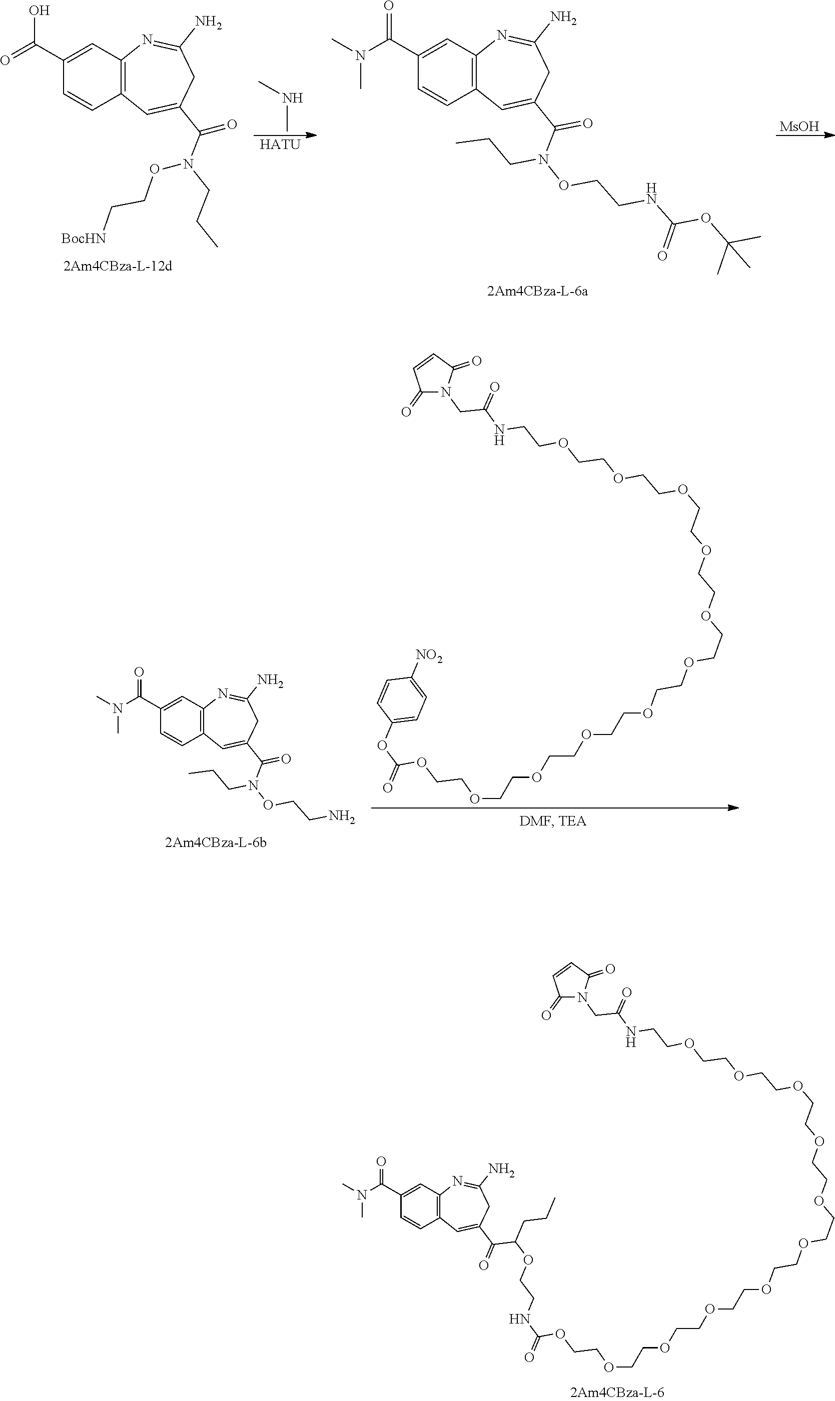

### Preparation of tert-butyl (2-((2-amino-8-(dimethylcarbamoyl)-N-propyl-3H-benzo[b]azepine-4-carboxamido)oxy)ethyl)carbamate, 2Am4CBza-L-6a

2-Amino-4-((2-((tert-butoxycarbonyl)amino)ethoxy)(propyl)carbamoyl)-3H-benzo[b]azepine-8-carboxylic acid, 2Am4CBza-L-12d was reacted with dimethylamine and HATU to give 2Am4CBza-L-6a. LC/MS [M+H] 474.27 (calculated); LC/MS [M+H] 474.45 (observed).

### Preparation of 2-amino-N4-(2-aminoethoxy)-N8,N8-dimethyl-N4-propyl-3H-benzo[b]azepine-4,8-dicarboxamide, 2Am4CBza-L-6b

2Am4CBza-L-6a was reacted with methanesulfonic acid to give 2Am4CBza-L-6b. LC/MS [M+H] 374.22 (calculated); LC/MS [M+H] 374.36 (observed).

### Preparation of 2Am4CBza-L-6

To a solution of 2Am4CBza-L-6b in DMF was added DIEA and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate and then stirred at 25° C. for 1 h. The mixture was quenched with TFA until pH=˜6. Then the mixture was filtered purified by prep-HPLC (column: Phenomenex Luna 80*30 mm*3 um; mobile phase: [water(TFA)-ACN]; B %: 5%-35%, 8 min) to give 2Am4CBza-L-6. LC/MS [M+H] 1038.52 (calculated); LC/MS [M+H] 1038.63 (observed).

### Example 7 Synthesis of 1-(2,5-dioxo-2,5-dihydro-1H-pyrrol-1-yl)-2-oxo-6,9,12,15,18,21,24,27,30,33-decaoxa-3-azapentatriacontan-35-yl (2-((2-amino-N-propyl-8-(pyrrolidine-1-carbonyl)-3H-benzo[b]azepine-4-carboxamido)oxy)ethyl)carbamate, 2Am4CBza-L-7

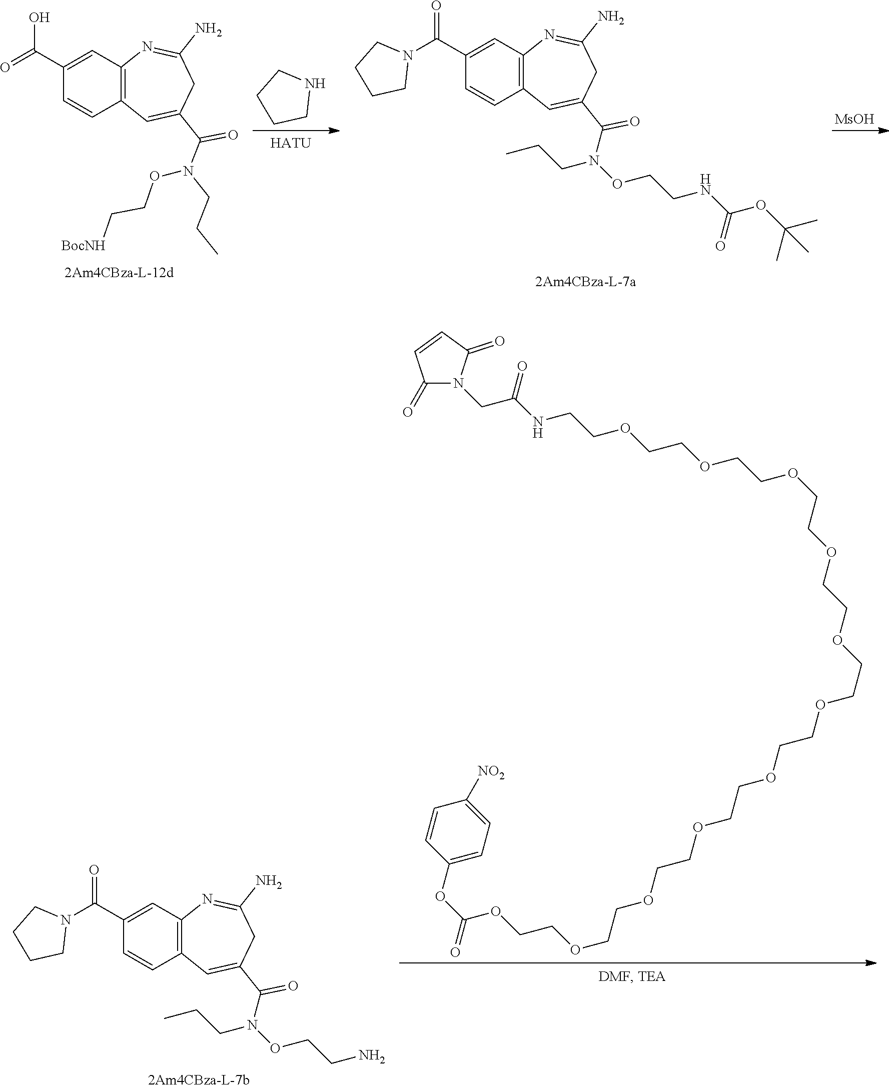

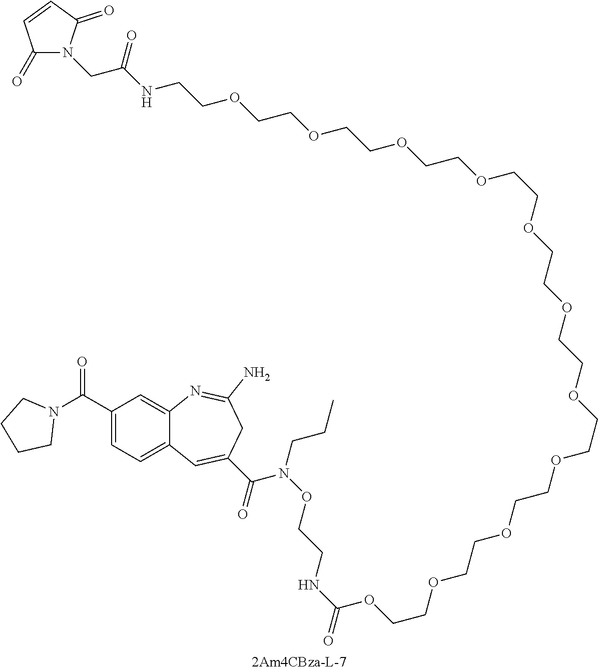 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCCC3)cc2N=C(N)C1]

### Preparation of tert-butyl (2-((2-amino-N-propyl-8-(pyrrolidine-1-carbonyl)-3H-benzo[b]azepine-4-carboxamido)oxy)ethyl)carbamate, 2Am4CBza-L-7a

2-Amino-4-((2-((tert-butoxycarbonyl)amino)ethoxy)(propyl)carbamoyl)-3H-5 benzo[b]azepine-8-carboxylic acid, 2Am4CBza-L-12d was reacted with pyrrolidine and HATU to give 2Am4CBza-L-7a. LC/MS [M+H] 500.29 (calculated); LC/MS [M+H] 500.48 (observed).

### Preparation of 2-amino-N-(2-aminoethoxy)-N-propyl-8-(pyrrolidine-1-carbonyl)-3H-benzo[b]azepine-4-carboxamide, 2Am4CBza-L-7b

2Am4CBza-L-7a was reacted with methanesulfonic acid to give 2Am4CBza-L-7b. LC/MS [M+H] 400.23 (calculated); LC/MS [M+H] 400.39 (observed).

### Preparation of 2Am4CBza-L-7

To a solution of 2Am4CBza-L-7b in DMF was added DIEA and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate and then stirred at 25° C. for 1 h. The mixture was quenched with TFA until pH=˜6. Then the mixture was filtered purified by prep-HPLC (column: Phenomenex Luna 80*30 mm*3 um; mobile phase: [water(TFA)-ACN]; B %: 5%-35%, 8 min) to give 2Am4CBza-L-7. LC/MS [M+H] 1064.54 (calculated); LC/MS [M+H] 1064.67 (observed).

### Example 8 Synthesis of 1-(2,5-dioxo-2,5-dihydro-1H-pyrrol-1-yl)-2-oxo-6,9,12,15,18,21,24,27,30,33-decaoxa-3-azapentatriacontan-35-yl (3-(2-amino-8-(dimethylcarbamoyl)-N-propyl-3H-benzo[b]azepine-4-carboxamido)propyl)carbamate, 2Am4CBza-L-8

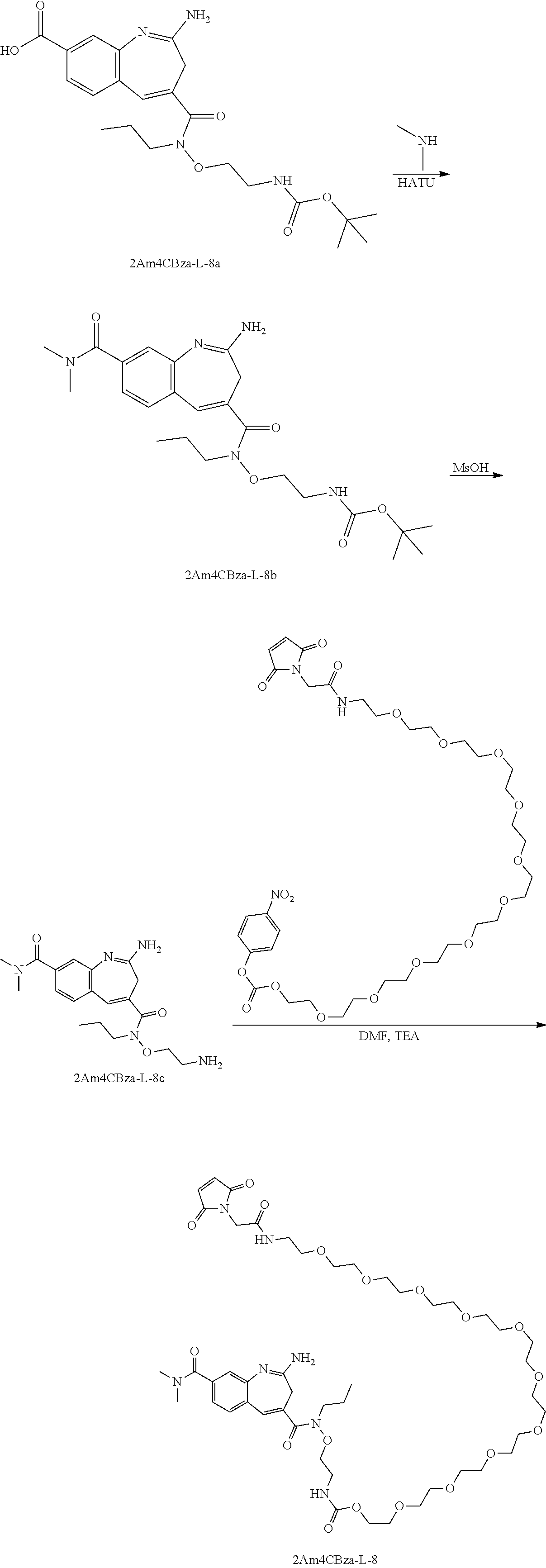

### Preparation of tert-butyl (3-(2-amino-8-(dimethylcarbamoyl)-N-propyl-3H-benzo[b]azepine-4-carboxamido)propyl)carbamate, 2Am4CBza-L-8b

2-Amino-4-((3-((tert-butoxycarbonyl)amino)propyl)(propyl)carbamoyl)-3H-benzo[b]azepine-8-carboxylic acid, 2Am4CBza-L-8a was reacted with dimethylamine and HATU to give 2Am4CBza-L-8b. LC/MS [M+H] 472.29 (calculated); LC/MS [M+H] 472.56 (observed).

### Preparation of 2-amino-N4-(3-aminopropyl)-N8,N8-dimethyl-N4-propyl-3H-benzo[b]azepine-4,8-dicarboxamide, 2Am4CBza-L-8c

2Am4CBza-L-8b was reacted with methanesulfonic acid to give 2Am4CBza-L-8c. LC/MS [M+H] 372.24 (calculated); LC/MS [M+H] 372.34 (observed).

### Preparation of 2Am4CBza-L-8

To a solution of 2Am4CBza-L-8c in DMF was added DIEA and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate and then stirred at 25° C. for 1 h. The mixture was quenched with TFA until pH=˜6. Then the mixture was filtered purified by prep-HPLC (column: Phenomenex Luna 80*30 mm*3 um; mobile phase: [water(TFA)-ACN]; B %: 5%-35%, 8 min) to give 2Am4CBza-L-8. LC/MS [M+H] 1036.54 (calculated); LC/MS [M+H] 1036.60 (observed).

### Example 9 Synthesis of 1-(2,5-dioxo-2,5-dihydro-1H-pyrrol-1-yl)-2-oxo-6,9,12,15,18,21,24,27,30,33-decaoxa-3-azapentatriacontan-35-yl (3-(2-amino-N-propyl-8-(pyrrolidine-1-carbonyl)-3H-benzo[b]azepine-4-carboxamido)propyl)carbamate, 2Am4CBza-L-9

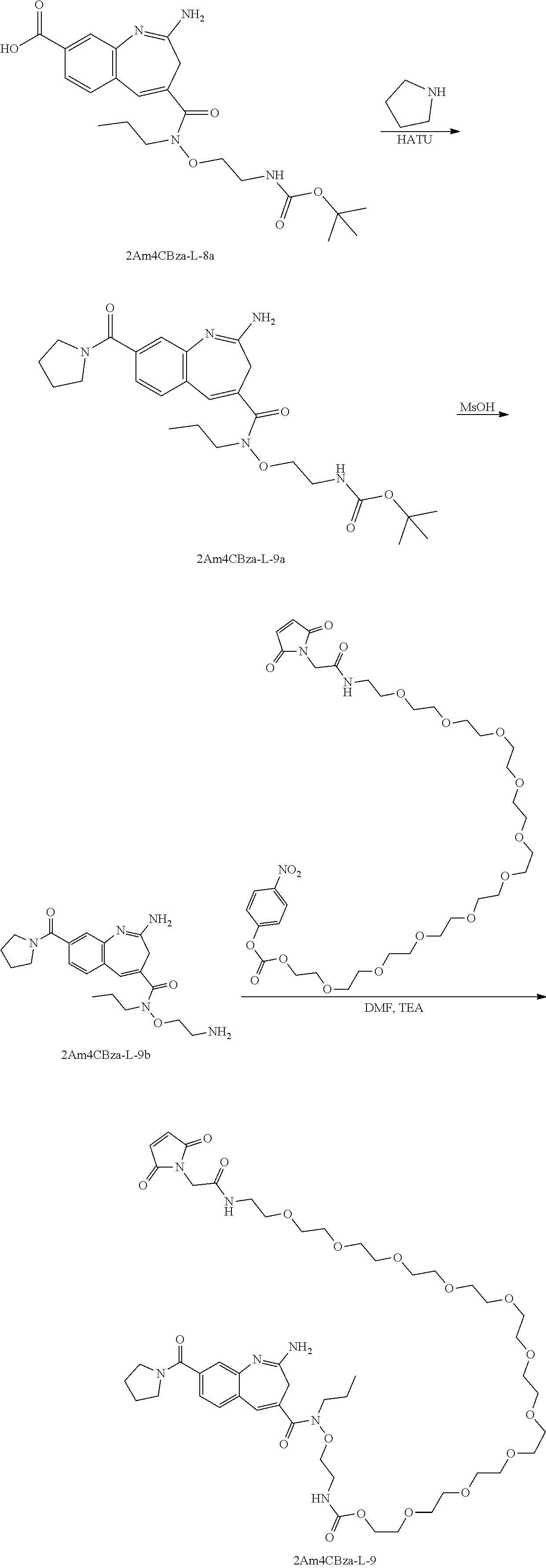

### Preparation of tert-butyl (3-(2-amino-N-propyl-8-(pyrrolidine-1-carbonyl)-3H-benzo[b]azepine-4-carboxamido)propyl)carbamate, 2Am4CBza-L-9a

2-Amino-4-((3-((tert-butoxycarbonyl)amino)propyl)(propyl)carbamoyl)-3H-benzo[b]azepine-8-carboxylic acid, 2Am4CBza-L-8a was reacted with pyrrolidine and HATU to give 2Am4CBza-L-9a. LC/MS [M+H] 498.31 (calculated); LC/MS [M+H] 498.44 (observed).

### Preparation of 2-amino-N-(3-aminopropyl)-N-propyl-8-(pyrrolidine-1-carbonyl)-3H-benzo[b]azepine-4-carboxamide, 2Am4CBza-L-9b

2Am4CBza-L-9a was reacted with methanesulfonic acid to give 2Am4CBza-L-9b. LC/MS [M+H] 398.26 (calculated); LC/MS [M+H] 398.37 (observed).

### Preparation of 1-(2,5-dioxo-2,5-dihydro-1H-pyrrol-1-yl)-2-oxo-6,9,12,15,18,21,24,27,30,33-decaoxa-3-azapentatriacontan-35-yl (3-(2-amino-N-propyl-8-(pyrrolidine-1-carbonyl)-3H-benzo[b]azepine-4-carboxamido)propyl)carbamate

To a solution of 2Am4CBza-L-9b in DMF was added DIEA and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate and then stirred at 25° C. for 1 h. The mixture was quenched with TFA until pH=˜6. Then the mixture was filtered purified by prep-HPLC (column: Phenomenex Luna 80*30 mm*3 um; mobile phase: [water(TFA)-ACN]; B %: 5%-35%, 8 min) to give 2Am4CBza-L-9. LC/MS [M+H] 1062.56 (calculated); LC/MS [M+H] 1062.20 (observed).

### Example 10 Synthesis of 4-[3-[2-[2-[2-[2-[2-[2-[2-[2-[2-[2-[2-[[2-amino-8-[(3R)-3-anilinopiperidine-1-carbonyl]-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoyloxy]-2,3,5,6-tetrafluoro-benzenesulfonic acid, 2Am4CBza-L-10

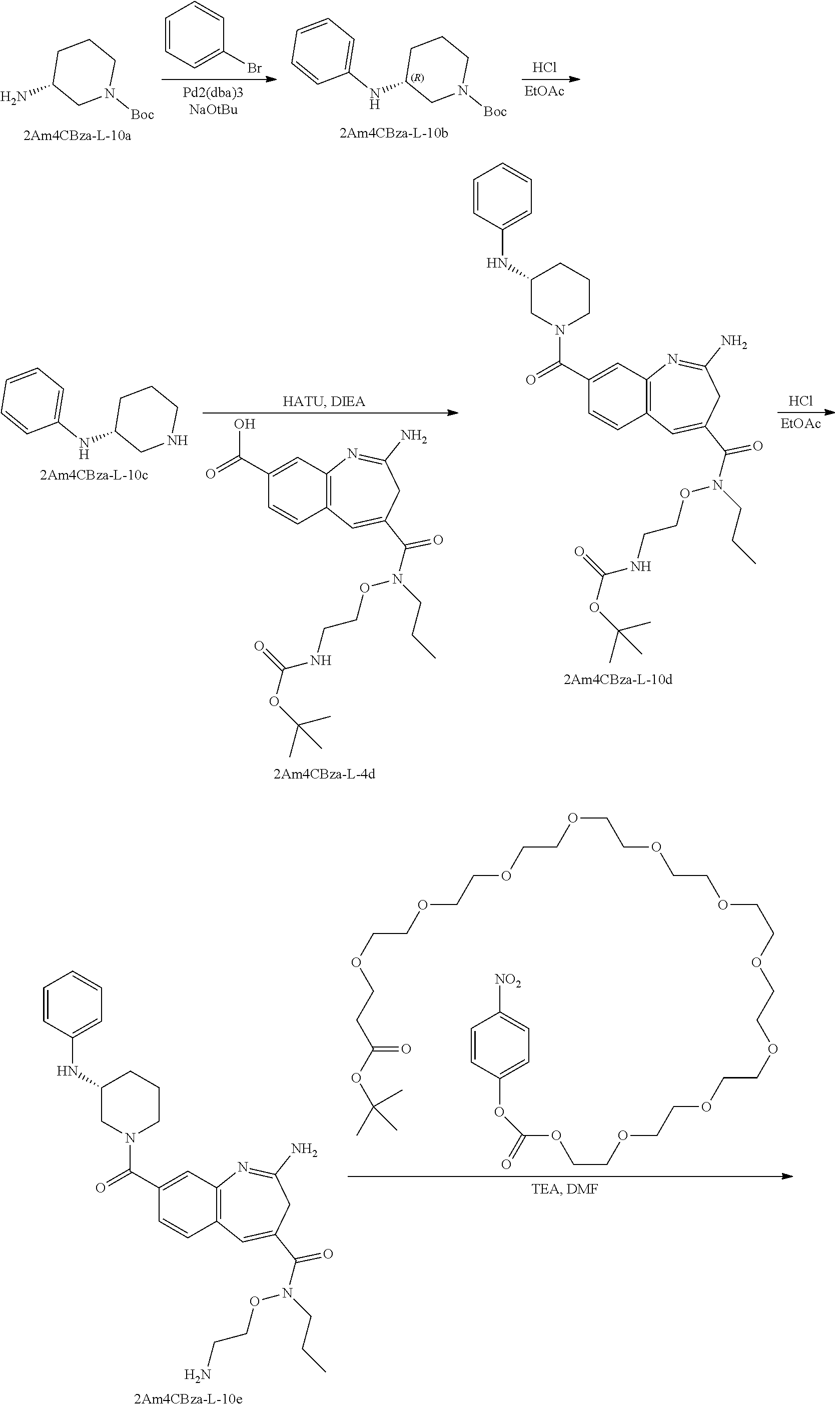

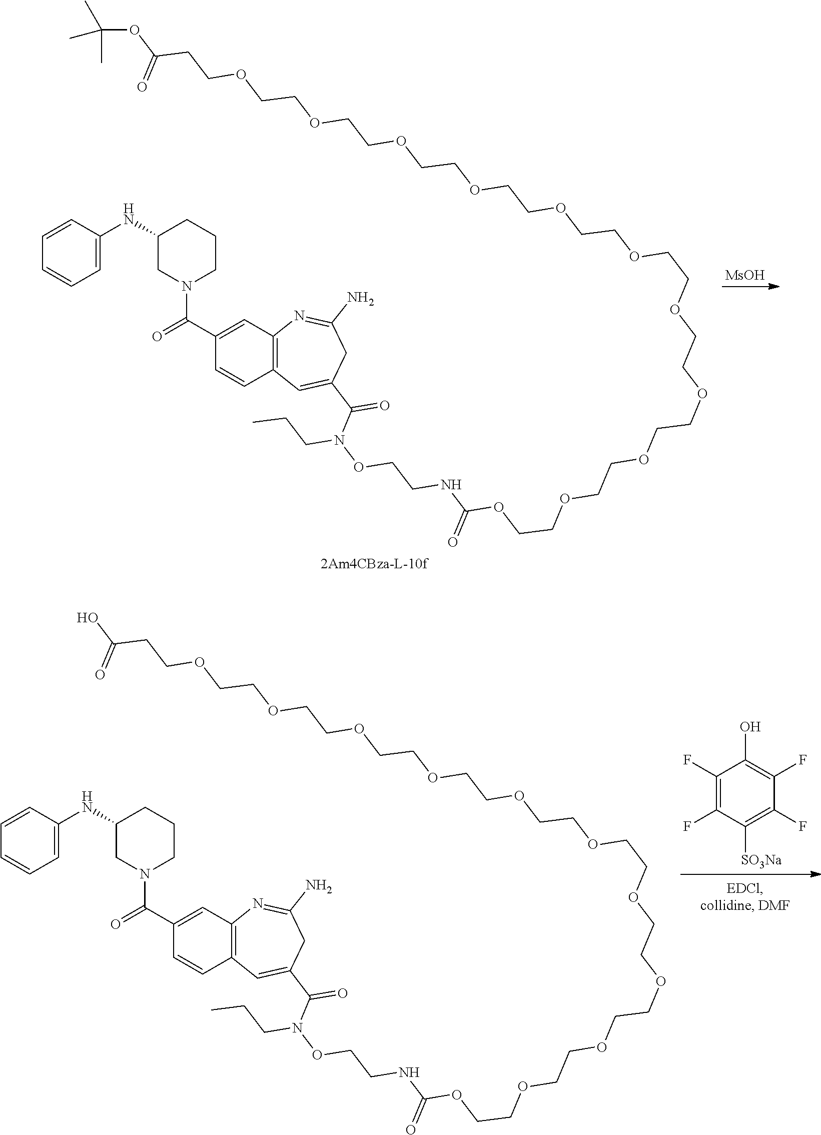

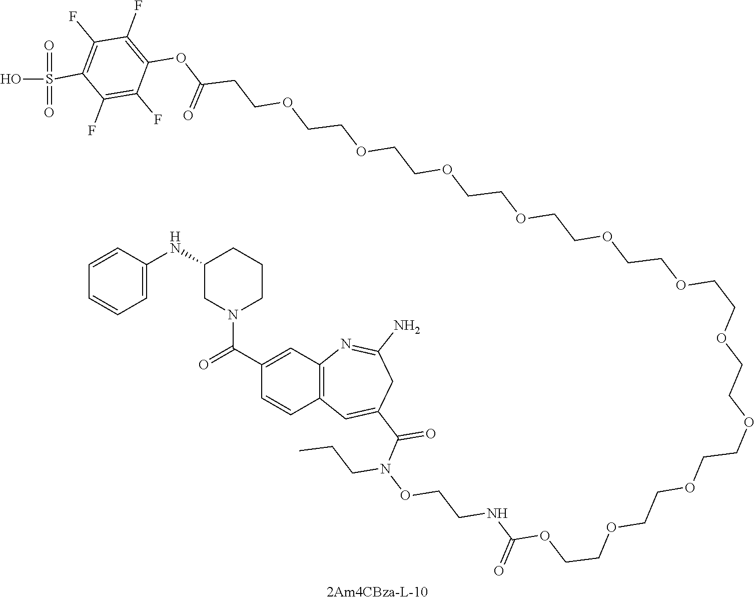 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCC[C@@H](Nc4ccccc4)C3)cc2N=C(N)C1]

### Preparation of tert-butyl (3R)-3-anilinopiperidine-1-carboxylate, 2Am4CBza-L-10b

To a solution of tert-butyl (3R)-3-aminopiperidine-1-carboxylate, 2Am4CBza-L-10a (1 g, 4.99 mmol, 1.0 eq) and bromobenzene (784 mg, 4.99 mmol, 526 uL, 1.0 eq) in toluene (10 mL) was added 2-(2-dicyclohexylphosphanylphenyl)-N,N-dimethyl-aniline (138 mg, 349 umol, 0.07 eq), sodium 2-methylpropan-2-olate, sodium tert-butoxide (576 mg, 5.99 mmol, 1.2 eq) and Pd₂(dba)₃ (228 mg, 250 umol, 0.05 eq)under N₂. The suspension was degassed under vacuum and purged with N2 several times, and then stirred 100° C. for 10 h. The reaction mixture was concentrated under reduced pressure to give a residue, the residue was diluted with H₂O (30 mL) and extracted with EtOAc (30 mL×3), the combined organic phase was washed with brine (15 mL), dried with anhydrous Na₂SO₄, filtered and concentrated in vacuum. The crude product was purified by silica gel chromatography eluted with Petroleum ether:Ethyl acetate=1:0-0:1. to give 2Am4CBza-L-10b (900 mg, 3.26 mmol, 65.2% yield) as colorless oil. ¹H NMR (CDCl₃, 400 MHz) δ7.22-7.17 (m, 2H), 6.77-6.67 (m, 3H), 4.09-3.96 (m, 1H), 3.77-3.70 (m, 1H), 3.42-3.34 (m, 1H), 3.12-2.82 (m, 1H), 3.02-2.82 (m, 1H), 2.04-1.98 (m, 1H), 1.79-1.71 (m, 1H), 1.63-1.52 (m, 4H), 1.46 (s, 9H). LC/MS [M+H] 277.2 (calculated); LC/MS [M+H] 277.2 (observed).

### Preparation of (3R)-N-phenylpiperidin-3-amine, 2Am4CBza-L-10c

To a solution of 2Am4CBza-L-10b (900 mg, 3.26 mmol, 1.0 eq) in EtOAc (5 mL) was added HCl/EtOAc (4 M, 16.3 mL, 20.0 eq), and then stirred at 20° C. for 2 h. The reaction mixture was concentrated in vacuum to give 2Am4CBza-L-10c (650 mg, 2.61 mmol, 80.1% yield, 2 HCl) as a white solid. ¹H NMR (CDCl₃, 400 MHz) δ7.52-7.47 (m, 2H), 7.43-7.32 (m, 3H), 3.96-3.85 (m, 1H), 3.56 (br d, J=12.0 Hz, 1H), 3.43-3.36 (m, 1H), 3.19-3.10 (m, 1H), 3.03 (dt, J=2.8, 12.2 Hz, 1H), 2.25-2.08 (m, 2H), 1.92-1.77 (m, 2H).

### Preparation of tert-butyl N-[2-[[2-amino-8-[(3R)-3-anilinopiperidine-1-carbonyl]-3H-1-benzazepine-4-carbonyl]-propyl-amino]oxyethyl]carbamate, 2Am4CBza-L-10d

To a solution of 2-amino-4-[2-(tert-butoxycarbonylamino)ethoxy-propyl-carbamoyl]-3H-1-benzazepine-8-carboxylic acid, 2Am4CBza-L-4d (250 mg, 560 umol, 1.0 eq) HATU (234 mg, 616 umol, 1.1 eq) and DIEA (290 mg, 2.24 mmol, 390 uL, 4.0 eq) in DMF (3 mL) was added (3R)-N-phenylpiperidin-3-amine (209 mg, 840 umol, 1.5 eq, 2 HCl) under N₂ at 0° C. The mixture was stirred 20° C. for 3 h. The reaction mixture was filtered and concentrated in vacuum. The residue was purified by prep-HPLC (column: Phenomenex Luna 80*30 mm*3 um; mobile phase: [water(0.1% TFA)-ACN]; B %: 10%-45%, 8 min) to give 2Am4CBza-L-10d (220 mg, 306 umol, 54.7% yield, TFA) as a white solid. ¹H NMR (MeOD, 400 MHz) δ7.58-7.52 (m, 1H), 7.52-7.43 (m, 1H), 7.43-7.38 (m, 1H), 7.36-7.30 (m, 1H), 7.24-7.16 (m, 1H), 6.97 (br t, J=7.2 Hz, 1H), 6.91-6.76 (m, 1H), 6.58-6.49 (m, 1H), 6.44 (br d, J=7.4 Hz, 1H), 3.93 (br s, 2H), 3.84-3.57 (m, 4H), 3.55-3.35 (m, 4H), 3.28-3.23 (m, 2H), 3.07-2.97 (m, 1H), 2.22-1.97 (m, 2H), 1.86-1.65 (m, 4H), 1.38 (s, 9H), 1.01-0.96 (m, 3H). LC/MS [M+H]605.3 (calculated); LC/MS [M+H] 605.3 (observed).

### Preparation of 2-amino-N-(2-aminoethoxy)-8-[(3R)-3-anilinopiperidine-1-carbonyl]-N-propyl-3H-1-benzazepine-4-carboxamide, 2Am4CBza-L-10e

To a solution of 2Am4CBza-L-Od (220 mg, 364 umol, 1.0 eq) in EtOAc (3 mL) was added HCl/EtOAc (4 M, 1.82 mL, 20.0 eq), and then stirred at 20° C. for 2 h. The reaction mixture was concentrated in vacuum to give 2Am4CBza-L-10e (170 mg, 294 umol, 80.9% yield, 2HCl) as a white solid. LC/MS [M+H] 505.3 (calculated); LC/MS [M+H] 505.2 (observed).

### Preparation of tert-butyl 3-[2-[2-[2-[2-[2-[2-[2-[2-[2-[2-[2-[[2-amino-8-[(3R)-3-anilinopiperidine-1-carbonyl]-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoate, 2Am4CBza-L-10f

To a solution of 2Am4CBza-L-10e (90 mg, 156 umol, 1.0 eq, 2 HCl) and DIEA (80.6 mg, 623 umol, 109 uL, 4.0 eq) in DMF (2 mL) was added tert-butyl 3-[2-[2-[2-[2-[2-[2-[2-[2-[2-[2-(4-nitrophenoxy)carbonyloxyethoxy]ethoxy]ethoxy]ethoxy]ethoxy]ethoxy]ethoxy]ethoxy]ethoxy]e thoxy]propanoate (117 mg, 156 umol, 1.0 eq) at 0° C., and then stirred 20° C. for 2 h. The reaction mixture was filtered and concentrated in vacuum, the residue was diluted with H₂O (10 mL) and extracted with EtOAc (10 mL×3), the combined organic phase was washed with brine (10 mL×3), dried with anhydrous Na₂SO₄, and concentrated in vacuum to give 2Am4CBza-L-10f (160 mg, 143 umol, 91.9% yield) was obtained as a white solid. LC/MS [M+H] 1117.6 (calculated); LC/MS [M+H] 1117.6 (observed).

### Preparation of 3-[2-[2-[2-[2-[2-[2-[2-[2-[2-[2-[2-[[2-amino-8-[(3R)-3-anilinopiperidine-1-carbonyl]-3H-1-benzazepine-4-carbonyl]-propyl-amino]oxyethylcarbamoyloxy]ethoxy]ethoxy]ethoxy]ethoxy]ethoxy]ethoxy]ethoxy]ethoxy]etho xy]ethoxy]propanoic acid, 2Am4CBza-L-10 g

To a solution of 2Am4CBza-L-10f (130 mg, 116 umol, 1.0 eq) in DCM (2 mL) was added methanesulfonic acid (111 mg, 1.16 mmol, 82.8 uL, 10.0 eq), and then stirred at 20° C. for 2 h under N₂ atmosphere. The reaction mixture was filtered and concentrated under reduced pressure to give a residue, The residue was purified by prep-HPLC (column: Phenomenex Luna 80*30 mm*3 um; mobile phase: [water(0.1% TFA)-ACN]; B %: 15%-45%, 8 min) to give 2Am4CBza-L-10 g (80 mg, 75.4 umol, 64.8% yield) as a light colorless oil. LC/MS [M+H]1061.6 (calculated); LC/MS [M+H] 1061.6 (observed).

### Preparation of 2Am4CBza-L-10

To a solution of 2Am4CBza-L-10 g (70 mg, 65.9 umol, 1.0 eq) and sodium 2,3,5,6-tetrafluoro-4-hydroxy-benzenesulfonate (70.7 mg, 264 umol, 4.0 eq) in DCM (1.5 mL) and DMA (0.5 mL) was added EDCI (50.6 mg, 264 umol, 4.0 eq), and then stirred at 20° C. for 1 h.

The reaction mixture was filtered and concentrated under reduced pressure to give a residue.

The residue was purified by prep-HPLC (column: Phenomenex Luna 80*30 mm*3 um; mobile phase: [water(TFA)-ACN]; B %: 15%-45%, 8 min) to give 2Am4CBza-L-10 (25.1 mg, 19.5 umol, 29.5% yield) as a white solid. ¹H NMR (MeOD-d₄, 400 MHz) δ7.73-7.63 (m, 1H), 7.60-7.42 (m, 3H), 7.41-7.25 (m, 3H), 7.15-7.03 (m, 1H), 6.72-6.60 (m, 1H), 4.00-3.94 (m, 2H), 3.90-3.82 (m, 4H), 3.74 (br t, J=7.2 Hz, 2H), 3.65-3.57 (m, 40H), 3.51 (br d, J=4.0 Hz, 2H), 3.45-3.38 (m, 2H), 3.27-3.14 (m, 2H), 3.00-2.96 (m, 2H), 2.21-2.08 (m, 1H), 1.81-1.69 (m, 4H), 0.99 (t, J=7.6 Hz, 3H). LC/MS [M+H] 1289.5 (calculated); LC/MS [M+H] 1289.5 (observed).

### Example 11 Synthesis of 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl N-[2-[[2-amino-8-[(3R)-3-anilinopiperidine-1-carbonyl]-3H-1-benzazepine-4-carbonyl]-propyl-amino]oxyethyl]carbamate, 2Am4CBza-L-11

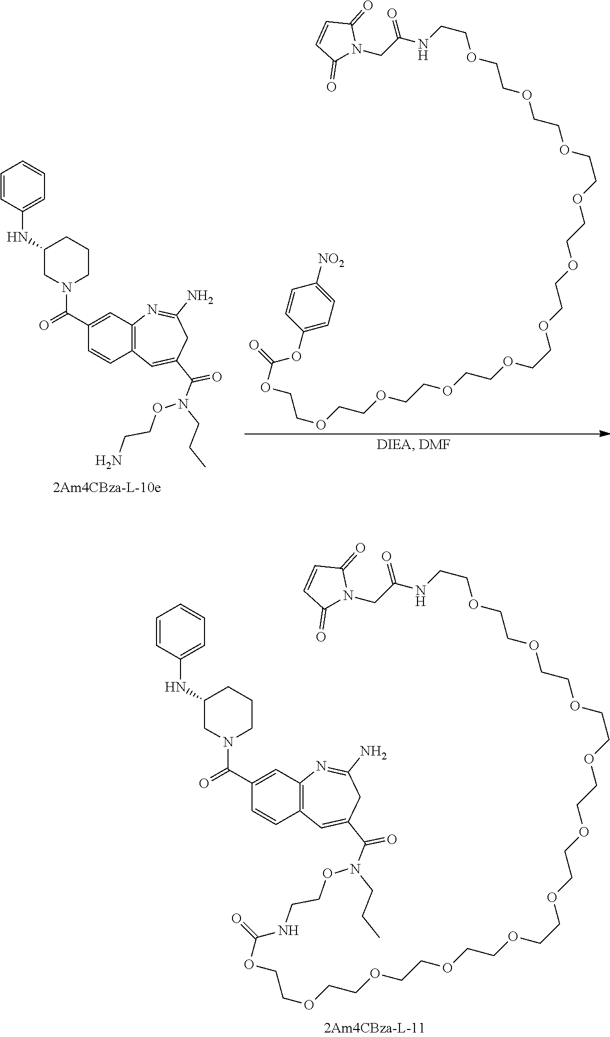 [SMILES: CCCN(OCCN)C(=O)C1=Cc2ccc(C(=O)N3CCC[C@@H](Nc4ccccc4)C3)cc2N=C(N)C1 ; O=C(CN1C(=O)C=CC1=O)NCCOCCOCCOCCOCCOCCOCCOCCOCCOCCOCCOC(=O)Oc1ccc([N+](=O)[O-])cc1 ; "2Am4CBza-L-10e" ; CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCC[C@@H](Nc4ccccc4)C3)cc2N=C(N)C1]

To a solution of 2-amino-N-(2-aminoethoxy)-8-[(3R)-3-anilinopiperidine-1-carbonyl]-N-propyl-3H-1-benzazepine-4-carboxamide, 2Am4CBza-L-10e (50.0 mg, 86.5 umol, 1.0 eq, 2HCl) in DMF (1.00 mL) was added DIEA (50.0 mg, 346 umol, 60.0 uL, 4.0 eq) and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]eth yl (4-nitrophenyl) carbonate (70.0 mg, 86.5 umol, 1.0 eq), and then stirred at 20° C. for 1 h. The mixture was filtered and purified by prep-HPLC (column: Phenomenex Luna 80*30 mm*3 um; mobile phase: [water(TFA)-ACN]; B %: 15%-40%, 8 min) to give 2Am4CBza-L-11 (23 mg, 17.9 umol, 20.7% yield, TFA) as light yellow oil. ¹H NMR (MeOD, 400 MHz) δ7.75-7.16 (m, 4H), 7.07-6.79 (m, 4H), 6.62-6.39 (m, 2H), 4.17 (s, 2H), 4.04-3.85 (m, 4H), 3.81-3.72 (m, 4H), 3.72-3.56 (m, 38H), 3.56-3.51 (m, 4H), 3.48-3.34 (m, 6H), 3.17-3.05 (m, 1H), 2.26-1.93 (m, 2H), 1.85-1.65 (m, 4H), 1.00 (t, J=7.6 Hz, 3H). LC/MS [M+H] 1169.6 (calculated); LC/MS [M+H] 1169.6 (observed).

### Example 12 Synthesis of 1-(2,5-dioxo-2,5-dihydro-1H-pyrrol-1-yl)-2-oxo-6,9,12,15,18,21,24,27,30,33-decaoxa-3-azapentatriacontan-35-yl (2-((2-amino-8-carbamoyl-N-propyl-3H-benzo[b]azepine-4-carboxamido)oxy)ethyl)carbamate, 2Am4CBza-L-12

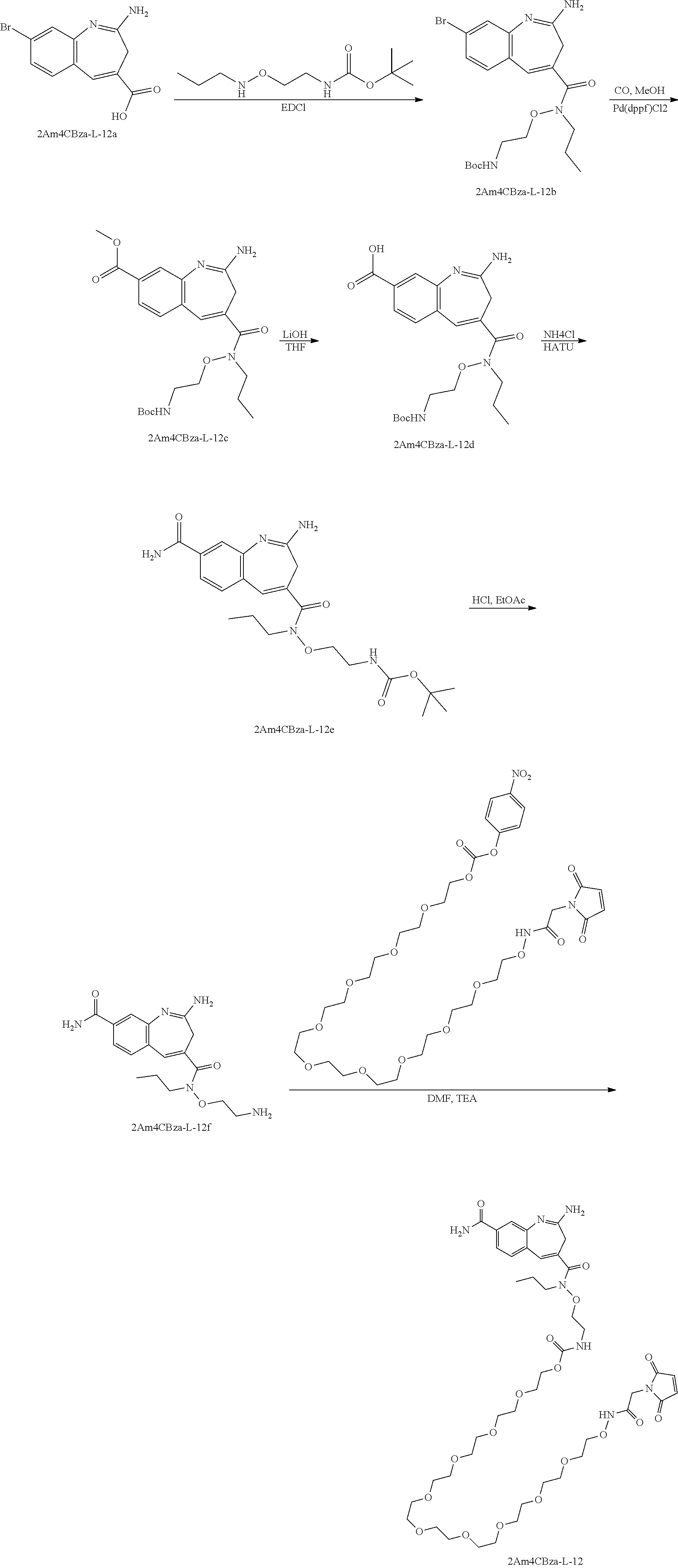

### Preparation of tert-butyl N-[2-[(2-amino-8-bromo-3H-1-benzazepine-4-carbonyl)-propyl-amino] oxyethyl]carbamate, 2Am4CBza-L-12b

To a mixture of 2-amino-8-bromo-3H-1-benzazepine-4-carboxylic acid, 2Am4CBza-L-12a (8.08 g, 28.7 mmol, 1.0 eq) in DCM (50 mL) and DMA (50 mL) was added methanesulfonic acid (2.76 g, 28.7 mmol, 2.05 mL, 1.0 eq), tert-butyl N-[2-(propylaminooxy)ethyl]carbamate (6.9 g, 31.6 mmol, 1.1 eq) and EDCI (22.0 g, 115 mmol, 4.0 eq) at 0° C. under N₂ and then stirred at this temperature for 2 h. The mixture was concentrated to remove DCM and diluted with water (100 mL). Then the pH of the mixture was adjusted to ˜8 with the addition of aq.Na2CO3. The aqueous phase was extracted with EtOAc (100 mL×3). The organic layer was washed with brine, dried over Na2SO4, filtered and concentrated. The crude product was triturated with MTBE (15 mL) and Petroleum ether (100 mL) at 0° C. for 0.5 hr to give 2Am4CBza-L-12b (11 g, 22.85 mmol, 79.52% yield) as a yellow solid. ¹H NMR (DMSO-d₆, 400 MHz) δ 7.27 (d, J=8.4 Hz, 1H), 7.14 (d, J=2.0 Hz, 1H), 7.08-7.01 (m, 2H), 7.01-6.87 (m, 2H), 6.85-6.82 (m, 1H), 3.80 (t, J=5.6 Hz, 2H), 3.57 (t, J=7.2 Hz, 2H), 3.09-3.05 (m, 2H), 2.76 (s, 2H), 1.67-1.56 (m, 2H), 1.31 (s, 9H), 0.87 (t, J=7.6 Hz, 3H). LC/MS [M+H] 481.1 (calculated); LC/MS [M+H] 481.1 (observed).

### Preparation of methyl 2-amino-4-[2-(tert-butoxycarbonylamino)ethoxy-propyl-carbamoyl]-3H-1-benzazepine-8-carboxylate, 2Am4CBza-L-12c

To a solution of 2Am4CBza-L-12b (8 g, 16.6 mmol, 1.0 eq) in MeOH (100 mL) was added Pd(dppf)Cl₂ (1.22 g, 1.66 mmol, 0.1 eq) and Et₃N (5.04 g, 49.9 mmol, 6.94 mL, 3.0 eq) under N₂. The suspension was degassed under vacuum and purged with CO several times. The mixture was stirred under CO (50 psi) at 80° C. for 12 hours. The mixture was filtered and concentrated. The residue was purified by silica gel chromatography (column height: 250 mm, diameter: 100 mm, 100-200 mesh silica gel, Ethyl acetate/MeOH=1/0, 10/1) to afford 2Am4CBza-L-12c (5 g, 10.86 mmol, 65.33% yield) as yellow solid. ¹H NMR (MeOD, 400 MHz) δ 7.81 (d, J=1.6 Hz, 1H), 7.63 (dd, J=1.6, 8.0 Hz, 1H), 7.46 (d, J=8.0 Hz, 1H), 7.27 (s, 1H), 3.94-3.86 (m, 5H), 3.73-3.69 (m, 2H), 3.31 (s, 2H), 3.23 (t, J=5.2 Hz, 2H), 1.81-1.70 (m, 2H), 1.35 (s, 9H), 0.97 (t, J=7.6 Hz, 3H). LC/MS [M+H] 461.2 (calculated); LC/MS [M+H]461.1 (observed).

### Preparation of 2-amino-4-[2-(tert-butoxycarbonylamino)ethoxy-propyl-carbamoyl]-3H-1-benzazepine-8-carboxylic acid, 2Am4CBza-L-12d

To a mixture of 2Am4CBza-L-12c (4 g, 8.69 mmol, 1.0 eq) in THF (30 mL) and H₂O (30 mL) was added LiOH·H₂O (547 mg, 13.0 mmol, 1.5 eq) in one portion at 25° C. and then stirred at this temperature for 2 h. The mixture was quenched with aq HCl (1 M) until pH=˜6. Then the mixture was diluted with water (30 mL) and extracted with EtOAc (50 mL×3). The organic layer washed with brine, dried over Na2SO4, filtered and concentrated. The mixture was purified by prep-HPLC (column: Phenomenex Luna 80*30 mm*3 um; mobile phase: [water(TFA)-ACN]; B %: 5%-40%, 8 min) to give 2Am4CBza-L-12d (3.3 g, 7.39 mmol, 85.09% yield) as yellow solid. ¹H NMR (MeOD, 400 MHz) δ 8.13 (s, 1H), 7.92 (d, J=7.6 Hz, 1H), 7.59 (d, J=8.0 Hz, 1H), 7.46 (s, 1H), 3.97-3.92 (m, 2H), 3.75 (t, J=7.2 Hz, 2H), 3.31-3.30 (m, 2H), 3.26 (t, J=5.2 Hz, 2H), 1.83-1.71 (m, 2H), 1.35 (s, 9H), 0.99 (t, J=7.6 Hz, 3H). LC/MS [M+H]447.2 (calculated); LC/MS [M+H] 447.2 (observed).

### Preparation of tert-butyl (2-((2-amino-8-carbamoyl-N-propyl-3H-benzo[b]azepine-4-carboxamido)oxy)ethyl)carbamate, 2Am4CBza-L-12e

2Am4CBza-L-12d was reacted with ammonium chloride and HATU to give 2Am4CBza-L-12e. LC/MS [M+H] 446.24 (calculated); LC/MS [M+H] 446.43 (observed).

### Preparation of 2-amino-N4-(2-aminoethoxy)-N4-propyl-3H-benzo[b]azepine-4,8-dicarboxamide, 2Am4CBza-L-12f

2Am4CBza-L-12e was reacted with hydrochloric acid in ethyl acetate to give 2Am4CBza-L-12f LC/MS [M+H] 346.19 (calculated); LC/MS [M+H] 346.35 (observed).

### Preparation of 2Am4CBza-L-12

To a solution of 2Am4CBza-L-12f in DMF was added DIEA and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate and then stirred at 25° C. for 1 h. The mixture was quenched with TFA until pH was about 6. Then the mixture was filtered purified by prep-HPLC (column: Phenomenex Luna 80*30 mm*3 um; mobile phase: [water(TFA)-ACN]; B %: 5%-35%, 8 min) to give 2Am4CBza-L-12. LC/MS [M+H] 1010.49 (calculated); LC/MS [M+H] 1010.71 (observed).

### Example 13 Synthesis of 1-(2,5-dioxo-2,5-dihydro-1H-pyrrol-1-yl)-2-oxo-6,9,12,15,18,21,24,27,30,33-decaoxa-3-azapentatriacontan-35-yl(3-(2-amino-8-(phenylcarbamoyl)-N-propyl-3H-benzo[b]azepine-4-carboxamido)propyl)carbamate, 2Am4CBza-L-13

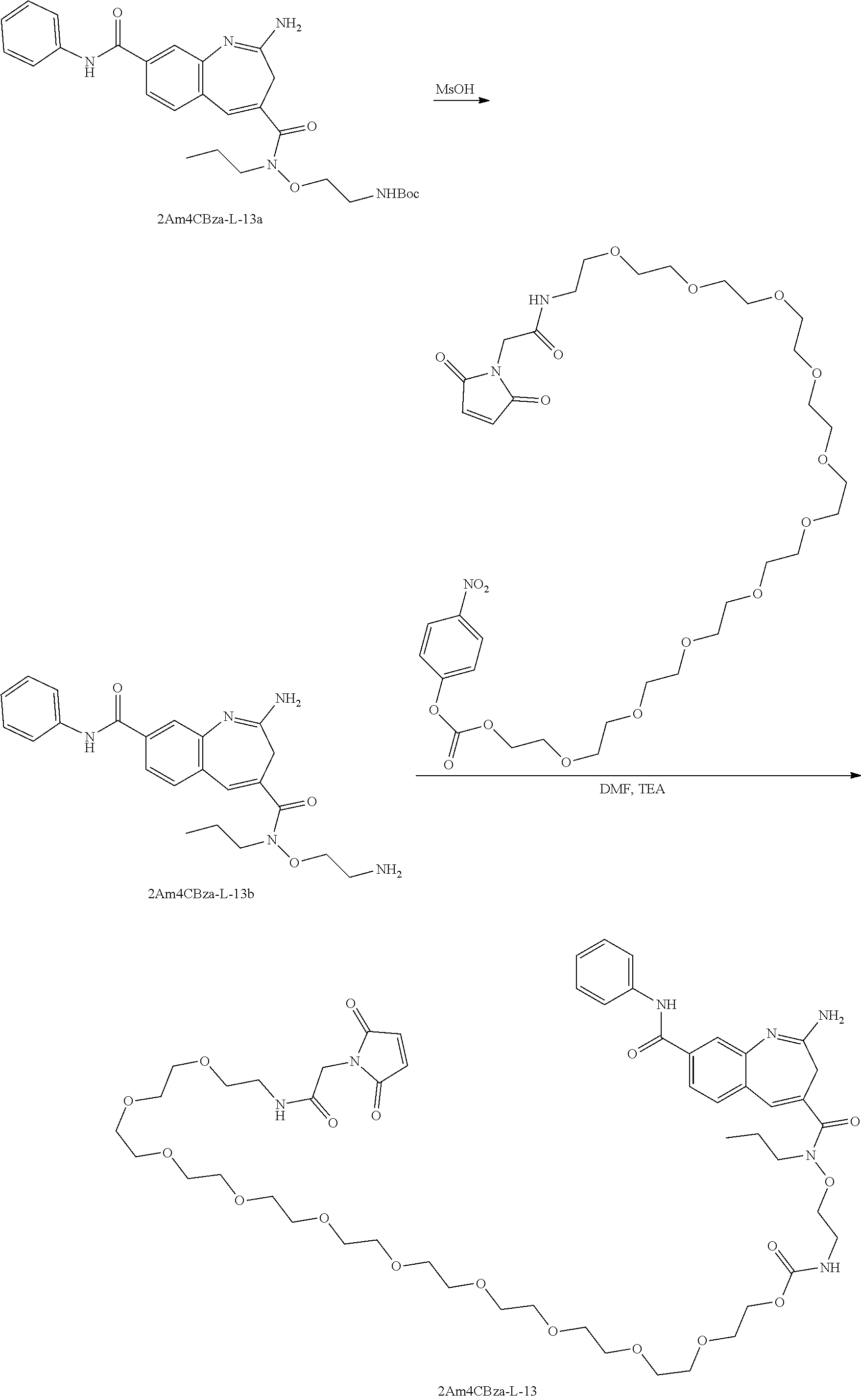 [SMILES: "MsOH" ; CCCN(OCCNC(=O)OC(C)(C)C)C(=O)C1=Cc2ccc(C(=O)Nc3ccccc3)cc2N=C(N)C1 ; CCCN(OCCN)C(=O)C1=Cc2ccc(C(=O)Nc3ccccc3)cc2N=C(N)C1 ; O=C(CN1C(=O)C=CC1=O)NCCOCCOCCOCCOCCOCCOCCOCCOCCOCCOCCOC(=O)Oc1ccc([N+](=O)[O-])cc1 ; CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)Nc3ccccc3)cc2N=C(N)C1]

### Preparation of 2-amino-N4-(2-aminoethoxy)-N8-phenyl-N4-propyl-3H-benzo[b]azepine-4,8-dicarboxamide, 2Am4CBza-L-13b

tert-Butyl (2-((2-amino-8-(phenylcarbamoyl)-N-propyl-3H-benzo[b]azepine-4-carboxamido)oxy)ethyl)carbamate, 2Am4CBza-L-13a was reacted with methanesulfonic acid to give 2Am4CBza-L-13b. LC/MS [M+H] 422.22 (calculated); LC/MS [M+H] 422.39 (observed).

### Preparation of 1-(2,5-dioxo-2,5-dihydro-1H-pyrrol-1-yl)-2-oxo-6,9,12,15,18,21,24,27,30,33-decaoxa-3-azapentatriacontan-35-yl (3-(2-amino-8-(phenylcarbamoyl)-N-propyl-3H-benzo[b]azepine-4-carboxamido)propyl)carbamate

To a solution of 2Am4CBza-L-13b in DMF was added DIEA and 2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl (4-nitrophenyl) carbonate and then stirred at 25° C. for 1 h. The mixture was quenched with TFA until pH was about 6. Then the mixture was filtered purified by prep-HPLC (column: Phenomenex Luna 80*30 mm*3 um; mobile phase: [water(TFA)-ACN]; B %: 5%-35%, 8 min) to give 2Am4CBza-L-13. LC/MS [M+H] 1086.52 (calculated); LC/MS [M+H] 1086.73 (observed).

### Example 17 Synthesis of 2-amino-N4-((40-(2,5-dioxo-2,5-dihydro-1H-pyrrol-1-yl)-4,39-dioxo-8,11,14,17,20,23,26,29,32,35-decaoxa-3,5,38-triazatetracontyl)oxy)-N8,N8-dimethyl-N4-propyl-3H-benzo[b]azepine-4,8-dicarboxamide, 2Am4CBza-L-17

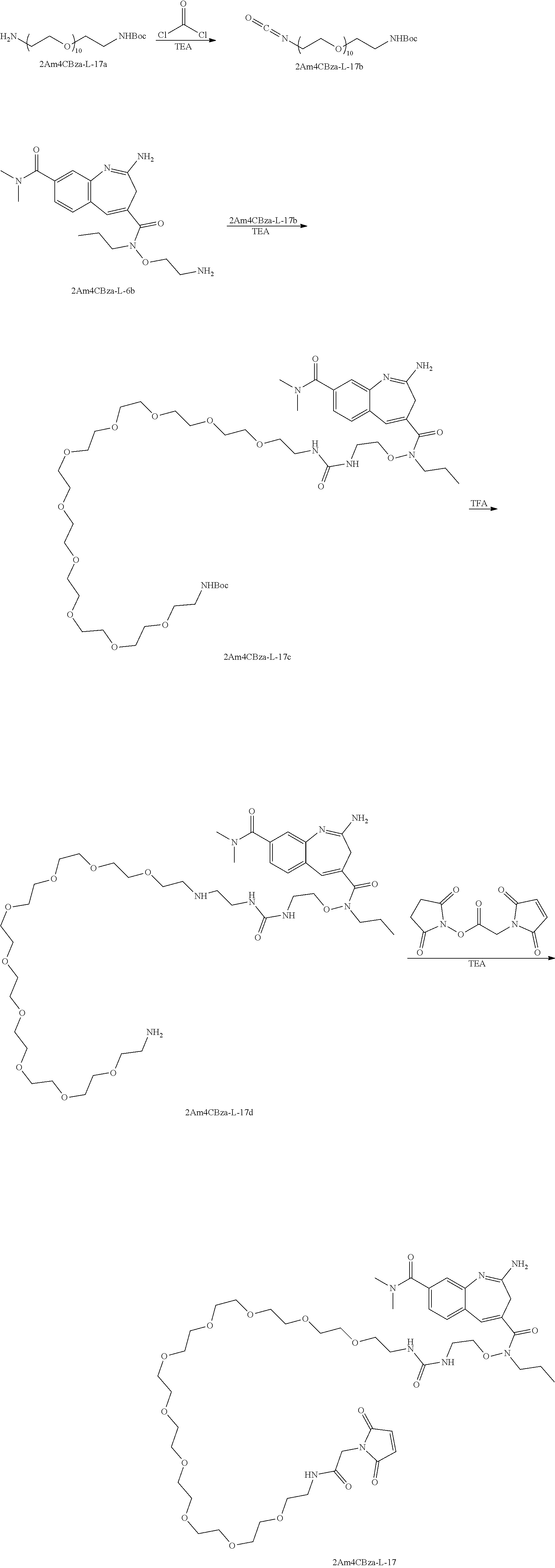

### Preparation of tert-butyl (32-isocyanato-3,6,9,12,15,18,21,24,27,30-decaoxadotriacontyl)carbamate, 2Am4CBza-L-17b

To a solution of tert-butyl (32-amino-3,6,9,12,15,18,21,24,27,30-decaoxadotriacontyl)carbamate, 2Am4CBza-L-17a (0.15 g, 0.25 mmol, 1 eq) in DCM was added TEA (0.348 ml, 2.5 mmol, 10 eq), followed by phosgene (0.892 ml as a 1.4 M solution in toluene, 0.25 mmol, 1 eq). The reaction mixture was monitored by LCMS, concentrated, and purified by reverse phase HPLC to give 2Am4CBza-L-17b (78 mg, 0.125 mmol, 50%). LC/MS [M+H] 627.37 (calculated); LC/MS [M+H] 627.64 (observed).

### Preparation of tert-butyl (39-(2-amino-8-(dimethylcarbamoyl)-3H-benzo[b]azepine-4-carbonyl)-34-oxo-3,6,9,12,15,18,21,24,27,30,38-undecaoxa-33,35,39-triazadotetracontyl)carbamate, 2Am4CBza-L-17c

To a mixture of 2-amino-N4-(2-aminoethoxy)-N8,N8-dimethyl-N4-propyl-3H-benzo[b]azepine-4,8-dicarboxamide, 2Am4CBza-L-6b (1 eq) and 2Am4CBza-L-17b (1 eq) in DMF was added TEA (10 eq). The reaction was stirred at room temperature, then diluted with water and purified by reverse phase HPLC to give 2Am4CBza-L-17c. LC/MS [M+H] 1000.58 (calculated); LC/MS [M+H] 1000.91 (observed).

### Preparation of 2-amino-N4-((37-amino-4-oxo-8,11,14,17,20,23,26,29,32,35-decaoxa-3,5-diazaheptatriacontyl)oxy)-N8,N8-dimethyl-N4-propyl-3H-benzo[b]azepine-4,8-dicarboxamide, 2Am4CBza-L-17d

2Am4CBza-L-17c (48 mg, 0.052 mmol, 1 eq) was dissolved in minimal TFA. After 15 minutes, the reaction mixture was concentrated to give 2Am4CBza-L-17d as a TFA salt. LC/MS [M+H] 900.53 (calculated); LC/MS [M+H] 900.83 (observed).

### Preparation of 2Am4CBza-L-17

To a solution of 2Am4CBza-L-17c (1 eq) in DMF was added TEA (12.8 eq) followed by 2,5-dioxopyrrolidin-1-yl 2-(2,5-dioxo-2,5-dihydro-1H-pyrrol-1-yl)acetate (1.28 eq). The reaction mixture was concentrated, diluted with 1% TFA in water, and purified by reverse phase HPLC to give 2Am4CBza-L-17. LC/MS [M+H] 1037.54 (calculated); LC/MS [M+H] 1037.84 (observed).

### Example 19 Synthesis of 2-amino-N4-[2-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]eth oxy]ethoxy]-N8,N8-dimethyl-N4-propyl-3H-1-benzazepine-4,8-dicarboxamide, 2Am4CBza-L-19

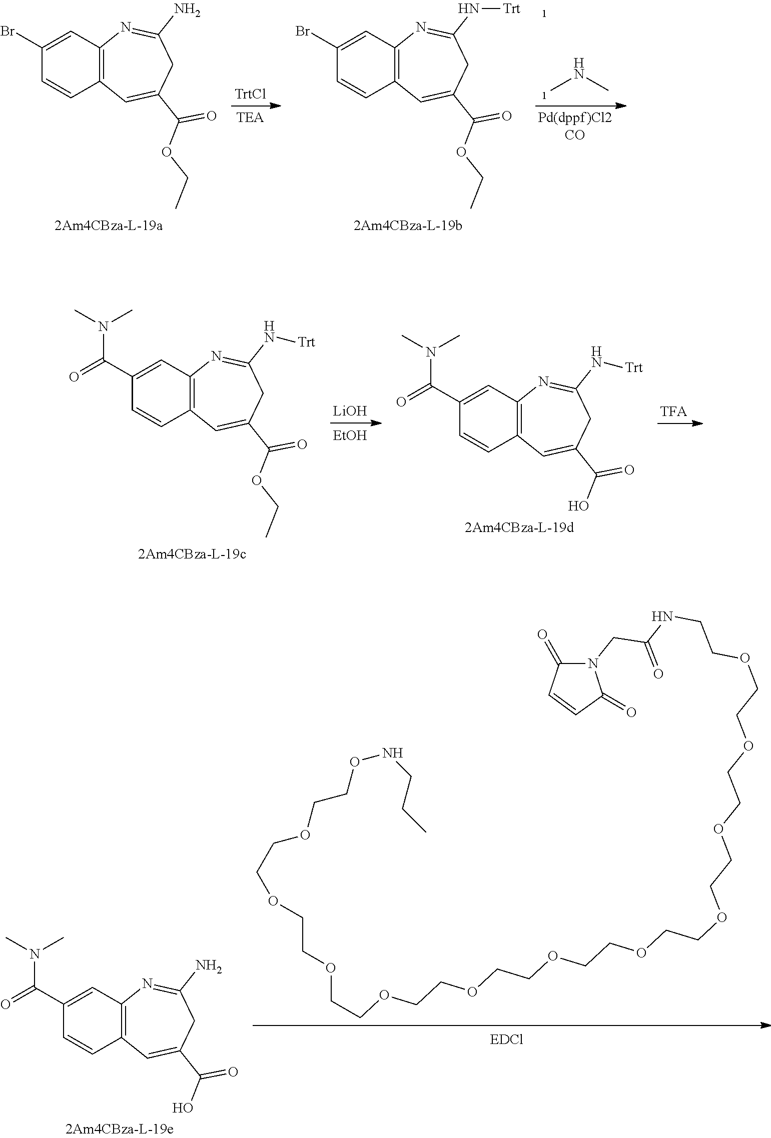

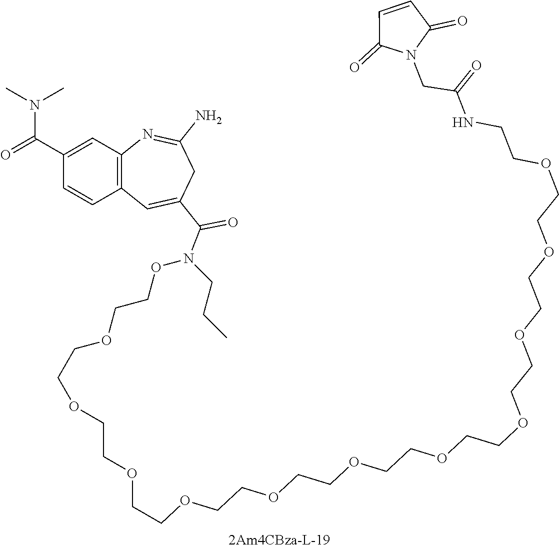 [SMILES: CCCN(OCCOCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1]

### Preparation of ethyl 8-bromo-2-(tritylamino)-3H-1-benzazepine-4-carboxylate, 2Am4CBza-L-19b

To a solution of ethyl 2-amino-8-bromo-3H-1-benzazepine-4-carboxylate, 2Am4CBza-L-19a (3 g, 9.70 mmol, 1 eq) in DCM (20 mL) was added TrtCl (4.06 g, 14.6 mmol, 1.5 eq) and Et₃N (2.95 g, 29.1 mmol, 4.05 mL, 3 eq). The mixture was stirred at 50° C. for 12 hrs. The result mixture was poured into ice-water (w/w=1/1) (30 mL) and stirred for 10 min. The aqueous phase was extracted with DCM (20 mL×3). The combined organic phase was dried with anhydrous Na₂SO₄, filtered and concentrated in vacuum. The crude product was triturated with MTBE at 25° C. for 5 min to afford 2Am4CBza-L-19b (4 g, 7.25 mmol, 74.75% yield) as yellow solid. LC/MS [M+H] 551.1 (calculated); LC/MS [M+H] 551.1 (observed).

### Preparation of ethyl 8-(dimethylcarbamoyl)-2-(tritylamino)-3H-1-benzazepine-4-carboxylate, 2Am4CBza-L-19c

A mixture of 2Am4CBza-L-19b (2 g, 3.63 mmol, 1 eq), N-methylmethanamine, dimethylamine (1.48 g, 18.2 mmol, 1.66 mL, 5 eq, HCl), Et₃N (3.67 g, 36.3 mmol, 5.05 mL, 10 eq), Pd(dppf)Cl₂ (266 mg, 363 umol, 0.1 eq) in DMF (30 mL) was degassed and purged with carbon monoxide, CO (3.63 mmol, 1 eq) for 3 times, and then the mixture was stirred at 80° C. for 12 hrs under CO (50 psi) atmosphere. The mixture was poured into ice-water (w/w=1/1) (30 mL) and stirred for 10 min. The aqueous phase was extracted with ethyl acetate (20 mL×3). The combined organic phase was washed with brine (20 mL×3), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 1/1) to afford 2Am4CBza-L-19c (900 mg, 1.66 mmol, 45.61% yield) as yellow solid. LC/MS [M+H] 544.2 (calculated); LC/MS [M+H] 544.4 (observed)

### Preparation of 8-(dimethylcarbamoyl)-2-(tritylamino)-3H-1-benzazepine-4-carboxylic acid, 2Am4CBza-L-19d

To a solution of 2Am4CBza-L-19c (800 mg, 1.47 mmol, 1 eq) in EtOH (15 mL) and H₂O (8 mL) was added LiOH·H₂O (247 mg, 5.89 mmol, 4 eq). The mixture was stirred at 20° C. for 12 hrs. The pH of the mixture was adjusted to ˜6 with HCl (1M), then concentrated in vacuum to remove EtOH. Water (20 mL) was added and desired white solid precipitated from the mixture, filtered to afford 2Am4CBza-L-19d (750 mg, 1.45 mmol, 98.85% yield) as white solid. ¹H NMR (400 MHz, DMSO-d₆) δ 8.19 (s, 1H), 7.69 (s, 1H), 7.35 (d, J=8.4 Hz, 1H), 7.29-7.19 (m, 12H), 7.17-7.09 (m, 3H), 6.83 (dd, J=1.6, 1.6 Hz, 1H), 6.35 (d, J=1.6 Hz, 1H), 3.00 (s, 2H), 2.91 (s, 3H), 2.80 (s, 3H). LC/MS [M+H] 516.2 (calculated); LC/MS [M+H] 516.4 (observed).

### Preparation of 2-amino-8-(dimethylcarbamoyl)-3H-1-benzazepine-4-carboxylic acid, 2Am4CBza-L-19e

To a solution of 2Am4CBza-L-19d (750 mg, 1.45 mmol, 1 eq) in DCM (10 mL) was added TFA (1.66 g, 14.6 mmol, 1.08 mL, 10 eq), and then stirred at 50° C. for 12 hrs. The mixture was concentrated in vacuum. The crude product was triturated with MTBE at 20° C. for 5 min to afford 2Am4CBza-L-19e (550 mg, 1.42 mmol, 97.62% yield, TFA) as white solid. ¹H NMR (400 MHz, DMSO-d₆) δ 7.90 (s, 1H), 7.73 (d, J=8.0 Hz, 1H), 7.44-7.36 (m, 2H), 3.50 (s, 2H), 3.00 (s, 3H), 2.93 (s, 3H). LC/MS [M+H] 274.1 (calculated); LC/MS [M+H] 274.1 (observed).

### Preparation of 2Am4CBza-L-19

To a solution of 2Am4CBza-L-19e (52.3 mg, 191 umol, 0.8 eq) and 2-(2,5-dioxopyrrol-1-yl)-N-[2-[2-[2-[2-[2-[2-[2-[2-[2-[2-[2-[2-(propylaminooxy)ethoxy]ethoxy]ethoxy]ethoxy]ethoxy]ethoxy]ethoxy]ethoxy]ethoxy]ethoxy]et hoxy]ethyl]acetamide (200 mg, 239 umol, 1.0 eq, MsOH) in DCM (3 mL) and DMA (1 mL) was added EDCI (229 mg, 1.20 mmol, 5.0 eq). The mixture was stirred at 25° C. for 0.5 hr. The reaction mixture was concentrated under reduced pressure to remove DCM. The residue was filtered and purified by prep-HPLC (TFA condition; column: Phenomenex Luna 80*30 mm*3 um; mobile phase: [water(TFA)-ACN]; B %: 10%-35%, 8 min) to give 2Am4CBza-L-19 (59.3 mg, 59.6 umol, 24.9% yield) as light yellow oil. ¹H NMR (MeOD, 400 MHz) δ7.65 (d, J=8.4 Hz, 1H), 7.48-7.44 (m, 2H), 7.36 (s, 1H), 6.89 (s, 2H), 4.17 (s, 2H), 4.07-4.03 (m, 2H), 3.75 (t, J=7.2 Hz, 2H), 3.68-3.53 (m, 40H), 3.50-3.45 (m, 2H), 3.44-3.35 (m, 4H), 3.28-3.23 (m, 2H), 3.13 (s, 3H), 3.05 (s, 3H), 1.78 (sxt, J=7.2 Hz, 2H), 1.01 (t, J=7.6 Hz, 3H). LC/MS [M+H] 995.5 (calculated); LC/MS [M+H] 995.5 (observed).

### Example 201 Preparation of Immunoconjugates (IC)

To prepare a lysine-conjugated Immunoconjugate, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, MO) or Zeba™ Spin Desalting Columns (Thermo Fisher Scientific). The eluates are then each adjusted to a concentration of about 1-10 mg/ml using the buffer and then sterile filtered. The antibody is pre-warmed to 20-30° C. and rapidly mixed with 2-20 (e.g., 7-10) molar equivalents of a tetrafluorophenyl (TFP) or sulfonic tetrafluorophenyl (sulfoTFP) ester, 2-amino-4-carboxamide-benzazepine-linker (2Am4CBza-L) compound of Formula II dissolved in dimethylsulfoxide (DMSO) or dimethylacetamide (DMA) to a concentration of 5 to 20 mM. The reaction is allowed to proceed for about 16 hours at 30° C. and the immunoconjugate (IC) is separated from reactants by running over two successive G-25 desalting columns or Zeba™ Spin Desalting Columns equilibrated in phosphate buffered saline (PBS) at pH 7.2 to provide the Immunoconjugate (IC) of Tables 3a and 3b. Adjuvant-antibody ratio (DAR) is determined by liquid chromatography mass spectrometry analysis using a C4 reverse phase column on an ACQUITY™ UPLC H-class (Waters Corporation, Milford, MA) connected to a XEVO™ G2-XS TOF mass spectrometer (Waters Corporation).

To prepare a cysteine-conjugated Immunoconjugate, an antibody is buffer exchanged into a conjugation buffer containing PBS, pH 7.2 with 2 mM EDTA using Zeba™ Spin Desalting Columns (Thermo Fisher Scientific). The interchain disulfides are reduced using 2-4 molar excess of Tris (2-carboxyethyl) phosphine (TCEP) or dithiothreitol (DTT) at 37° C. for 30 min-2 hours. Excess TCEP or DTT was removed using a Zeba™ Spin Desalting column pre-equilibrated with the conjugation buffer. The concentration of the buffer-exchanged antibody was adjusted to approximately 5 to 20 mg/ml using the conjugation buffer and sterile-filtered. The maleimide-2Am4CBza-L compound is either dissolved in dimethylsulfoxide (DMSO) or dimethylacetamide (DMA) to a concentration of 5 to 20 mM. For conjugation, the antibody is mixed with 10 to 20 molar equivalents of maleimide-2Am4CBza-L. In some instances, additional DMA or DMSO up to 20% (v/v), was added to improve the solubility of the maleimide-2Am4CBza-L in the conjugation buffer. The reaction is allowed to proceed for approximately 30 min to 4 hours at 20° C. The resulting conjugate is purified away from the unreacted maleimide-2Am4CBza-L using two successive Zeba™ Spin Desalting Columns. The columns are pre-equilibrated with phosphate-buffered saline (PBS), pH 7.2. Adjuvant to antibody ratio (DAR) is estimated by liquid chromatography mass spectrometry analysis using a C4 reverse phase column on an ACQUITY™ UPLC H-class (Waters Corporation, Milford, MA) connected to a XEVO™ G2-XS TOF mass spectrometer (Waters Corporation).

For conjugation, the antibody may be dissolved in an aqueous buffer system known in the art that will not adversely impact the stability or antigen-binding specificity of the antibody. Phosphate buffered saline may be used. The 2Am4CBza-L compound is dissolved in a solvent system comprising at least one polar aprotic solvent as described elsewhere herein. In some such aspects, 2Am4CBza-L is dissolved to a concentration of about 5 mM, about 10 mM, about 20 mM, about 30 mM, about 40 mM or about 50 mM, and ranges thereof such as from about 5 mM to about 50 mM or from about 10 mM to about 30 mM in pH 8 Tris buffer (e.g., 50 mM Tris). In some aspects, the 2-amino-4-carboxamide-benzazepine-linker intermediate is dissolved in DMSO (dimethylsulfoxide), DMA (dimethylacetamide), acetonitrile, or another suitable dipolar aprotic solvent.

Alternatively in the conjugation reaction, an equivalent excess of 2Am4CBza-L solution may be diluted and combined with antibody solution. The 2Am4CBza-L solution may suitably be diluted with at least one polar aprotic solvent and at least one polar protic solvent, examples of which include water, methanol, ethanol, n-propanol, and acetic acid. The molar equivalents of 2Am4CBza-L intermediate to antibody may be about 1.5:1, about 3:1, about 5:1, about 10:1, about 15:1, or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS. The conjugation reaction is typically complete in a range from about 1 hour to about 16 hours. After the reaction is complete, a reagent may be added to the reaction mixture to quench the reaction. If antibody thiol groups are reacting with a thiol-reactive group such as maleimide of the 2Am4CBza-L linker intermediate, unreacted antibody thiol groups may be reacted with a capping reagent. An example of a suitable capping reagent is ethylmaleimide.

Following conjugation, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, tangential flow filtration, and combinations thereof. For instance, purification may be preceded by diluting the immunoconjugate, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 202 HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4×10⁴ cells/well with substrate containing HEK detection medium and immunostimulatory 2-amino-4-carboxamide-benzazepine compounds, such as those of Table 1. Activity was measured using a plate reader at 620-655 nm wavelength.

### Example 203 Assessment of Immunoconjugate Activity In Vitro

This example shows that Immunoconjugates of the invention, including those of Tables 3a and 3b, are effective at eliciting immune activation, and therefore are useful for the treatment of cancer.
a) Isolation of Human Antigen Presenting Cells: Human myeloid antigen presenting cells (APCs) were negatively selected from human peripheral blood obtained from healthy blood donors (Stanford Blood Center, Palo Alto, California) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >90% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.
b) Myeloid APC Activation Assay: 2×10⁵ APCs are incubated in 96-well plates (Corning, Corning, NY) containing iscove's modified dulbecco's medium, IMDM (Lonza) supplemented with 10% FBS, 100 U/mL penicillin, 100 μg/mL (micrograms per milliliter) streptomycin, 2 mM L-glutamine, sodium pyruvate, non-essential amino acids, and where indicated, various concentrations of unconjugated (naked) antibodies and immunoconjugates (IC) of the invention, including those of Tables 3a and 3b, (as prepared according to the Example above). Cell-free supernatants are analyzed after 18 hours via ELISA to measure TNFα secretion as a readout of a proinflammatory response.
c) PBMC Activation Assay: Human peripheral blood mononuclear cells were isolated from human peripheral blood obtained from healthy blood donors (Stanford Blood Center, Palo Alto, California) by density gradient centrifugation. PBMCs were incubated in 96-well plates (Corning, Corning, NY) in a co-culture with CEA-expressing tumor cells (e.g. MKN-45, HPAF-II) at a 10:1 effector to target cell ratio. Cells were stimulated with various concentrations of unconjugated (naked) antibodies and immunoconjugates of the invention (as prepared according to the Example above). Cell-free supernatants were analyzed by cytokine bead array using a LegendPlex™ kit according to manufacturer's guidelines (BioLegend®, San Diego, CA).
d) Isolation of Human Conventional Dendritic Cells: Human conventional dendritic cells (cDCs) were negatively selected from human peripheral blood obtained from healthy blood donors (Stanford Blood Center, Palo Alto, California) by density gradient centrifugation.

Briefly, cells are first enriched by using a ROSETTESEP™ Human CD3 Depletion Cocktail (Stem Cell Technologies, Vancouver, Canada) to remove T cells from the cell preparation. cDCs are then further enriched via negative selection using an EASYSEP™ Human Myeloid DC Enrichment Kit (Stem Cell Technologies).
e) cDC Activation Assay: 8×10⁴ APCs were co-cultured with tumor cells expressing the ISAC target antigen at a 10:1 effector (cDC) to target (tumor cell) ratio. Cells were incubated in 96-well plates (Corning, Corning, NY) containing RPMI-1640 medium supplemented with 10% FBS, and where indicated, various concentrations of the indicated immunoconjugate of the invention (as prepared according to the example above). Following overnight incubation of about 18 hours, cell-free supernatants were collected and analyzed for cytokine secretion (including TNFα) using a BioLegend LEGENDPLEX cytokine bead array.

Activation of myeloid cell types can be measured using various screen assays in addition to the assay described in which different myeloid populations are utilized. These may include the following: monocytes isolated from healthy donor blood, M-CSF differentiated Macrophages, GM-CSF differentiated Macrophages, GM-CSF+IL-4 monocyte-derived Dendritic Cells, conventional Dendritic Cells (cDCs) isolated from healthy donor blood, and myeloid cells polarized to an immunosuppressive state (also referred to as myeloid derived suppressor cells or MDSCs). Examples of MDSC polarized cells include monocytes differentiated toward immunosuppressive state such as M2a MΦ (IL4/IL13), M2c MΦ (IL10/TGFb), GM-CSF/IL6 MDSCs and tumor-educated monocytes (TEM). TEM differentiation can be performed using tumor-conditioned media (e.g. 786.0, MDA-MB-231, HCC1954). Primary tumor-associated myeloid cells may also include primary cells present in dissociated tumor cell suspensions (Discovery Life Sciences).

Assessment of activation of the described populations of myeloid cells may be performed as a mono-culture or as a co-culture with cells expressing the antigen of interest which the immunoconjugate (IC) may bind to via the CDR region of the antibody. Following incubation for 18-48 hours, activation may be assessed by upregulation of cell surface costimulatory molecules using flow cytometry or by measurement of secreted proinflammatory cytokines. For cytokine measurement, cell-free supernatant is harvested and analyzed by cytokine bead array (e.g. LegendPlex from Biolegend) using flow cytometry.

All references, including publications, patent applications, and patents, cited herein are hereby incorporated by reference to the same extent as if each reference were individually and specifically indicated to be incorporated by reference and were set forth in its entirety herein.

1. An immunoconjugate comprising an antibody covalently attached to one or more 2-amino-4-carboxamide-benzazepine moieties by a linker, and having Formula I: 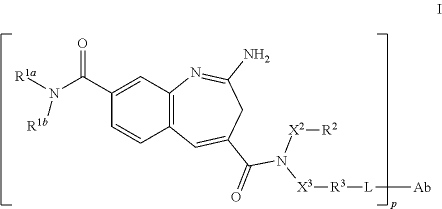
or a pharmaceutically acceptable salt thereof,
wherein:
Ab is the antibody;
p is an integer from 1 to 8;
X² and X³ are independently selected from the group consisting of a bond, C(═O), C(═O)N(R⁵), O, N(R⁵), S, S(O)₂, and S(O)₂N(R⁵);
R^{1a}, R^{1b}, and R² are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl; or R^{1a} and R^{1b} form a five- or six-membered heterocyclyl ring;
R³ is selected from the group consisting of:
—(C₁-C₁₂ alkyldiyl)-N(R⁵)—*;
—(C₁-C₁₂ alkyldiyl)-N(R⁵)—C(═O)*;
—(C₁-C₁₂ alkyldiyl)-N(R⁵)—C(═O)O—(C₃-C₁₂ carbocyclyldiyl)-*;
—(C₁-C₁₂ alkyldiyl)-N(R⁵)—(C₁-C₂₀ heteroaryldiyl)-*;
—(C₁-C₁₂ alkyldiyl)-N(R⁵)—(C₁-C₂₀ heteroaryldiyl)-(C₁-C₁₂ alkyldiyl)-*;
—(C₁-C₁₂ alkyldiyl)-N(R⁵)—S(O₂)—*;
—(C₁-C₁₂ alkyldiyl)-OC(═O)—(C₂-C₉ heterocyclyldiyl)-*;
—(C₁-C₁₂ alkyldiyl)-O—*;
—(C₁-C₁₂ alkyldiyl)-(C₃-C₁₂ carbocyclyldiyl)-*;
—(C₁-C₁₂ alkyldiyl)-(C₆-C₂₀ aryldiyl)-*;
—(C₁-C₁₂ alkyldiyl)-(C₆-C₂₀ aryl)-(C₁-C₁₂ alkyldiyl)-N(R⁵)—*;
—(C₁-C₁₂ alkyldiyl)-(C₂-C₉ heterocyclyldiyl)-(C₁-C₁₂ alkyldiyl)-N(R⁵)—*;
—(C₁-C₁₂ alkyldiyl)-(C₁-C₂₀ heteroaryldiyl)-N(R⁵)—*;
—(C₁-C₁₂ alkyldiyl)-(C₁-C₂₀ heteroaryldiyl)-*;
—(C₁-C₁₂ alkyldiyl)-(C₁-C₂₀ heteroaryldiyl)-(C₁-C₁₂ alkyldiyl)-*;
—(C₁-C₁₂ alkyldiyl)-(C₁-C₂₀ heteroaryldiyl)-(C₁-C₁₂ alkyldiyl)-N(R⁵)—*;
—(C₃-C₁₂ carbocyclyldiyl)-*;
—(C₃-C₁₂ carbocyclyldiyl)-(C₁-C₁₂ alkyldiyl)-N(R⁵)—*;
—(C₃-C₁₂ carbocyclyldiyl)-(C₁-C₁₂ alkyldiyl)-N(R⁵)—*;
—(C₃-C₁₂ carbocyclyldiyl)-NR⁵—C(═NR^{5a})—N(R⁵)—*;
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)—C(═NR^{5a})—N(R⁵)—*;
—(C₂-C₂₀ heterocyclyldiyl)-*;
—(C₂-C₉ heterocyclyldiyl)-(C₁-C₁₂ alkyldiyl)-N(R⁵)—*;
—(C₂-C₉ heterocyclyldiyl)-N(R⁵)—C(═NR^{5a})—N(R⁵)—*;
—(C₁-C₂₀ heteroaryldiyl)-*;
—(C₁-C₂₀ heteroaryldiyl)-(C₁-C₁₂ alkyldiyl)-N(R⁵)—*;
—(C₁-C₂₀ heteroaryldiyl)-(C₁-C₁₂ alkyldiyl)-O—*; and
—(C₁-C₂₀ heteroaryldiyl)-N(R⁵)—C(═NR^{5a})—N(R⁵)—*;
where the asterisk * indicates the attachment site of the linker L;
or R² and R³ together form a 5- or 6-membered heterocyclyl ring;
R⁵ is independently selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
L is selected from the group consisting of:
—C(═O)-PEG-;
—C(═O)-PEG-C(═O)N(R⁶)—(C₁-C₁₂ alkyldiyl)-C(═O)-Gluc-;
—C(═O)-PEG-O—;
—C(═O)-PEG-O—C(═O)—;
—C(═O)-PEG-C(═O)—;
—C(═O)-PEG-C(═O)-PEP-;
—C(═O)-PEG-N(R⁶)—;
—C(═O)-PEG-N(R⁶)—C(═O)—;
—C(═O)-PEG-N(R⁶)-PEG-C(═O)-PEP-;
—C(═O)-PEG-N⁺(R⁶)₂-PEG-C(═O)-PEP-;
—C(═O)-PEG-C(═O)-PEP-N(R⁶)—(C—C₁₂ alkyldiyl)-;
—C(═O)-PEG-C(═O)-PEP-N(R⁶)—(C₁-C₁₂ alkyldiyl)N(R⁶)C(═O)—(C₂-C₅ monoheterocyclyldiyl)-;
—C(═O)-PEG-SS-(C₁-C₁₂ alkyldiyl)-OC(═O)—;
—C(═O)-PEG-SS-(C₁-C₁₂ alkyldiyl)-C(═O)—;
—C(═O)—(C₁-C₁₂ alkyldiyl)-C(═O)-PEP-;
—C(═O)—(C₁-C₁₂ alkyldiyl)-C(═O)-PEP-N(R⁶)—(C₁-C₁₂ alkyldiyl)-;
—C(═O)—(C₁-C₁₂ alkyldiyl)-C(═O)-PEP-N(R⁶)—(C₁-C₁₂ alkyldiyl)-N(R⁵)—C(═O);
—C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
-succinimidyl-(CH₂)ₘ—C(═O)N(R⁶)-PEG-;
-succinimidyl-(CH₂)ₘ—C(═O)N(R⁶)-PEG-C(═O)N(R⁶)—(C₁-C₁₂ alkyldiyl)-C(═O)-Gluc-;
-succinimidyl-(CH₂)ₘ—C(═O)N(R⁶)-PEG-O—;
-succinimidyl-(CH₂)ₘ—C(═O)N(R⁶)-PEG-O—C(═O)—;
-succinimidyl-(CH₂)ₘ—C(═O)N(R⁶)-PEG-C(═O)—;
-succinimidyl-(CH₂)ₘ—C(═O)N(R⁶)-PEG-N(R⁵)—;
-succinimidyl-(CH₂)ₘ—C(═O)N(R⁶)-PEG-N(R⁵)—C(═O)—;
-succinimidyl-(CH₂)ₘ—C(═O)N(R⁶)-PEG-C(═O)-PEP-;
-succinimidyl-(CH₂)ₘ—C(═O)N(R⁶)-PEG-SS-(C₁-C₁₂ alkyldiyl)-OC(═O)—;
-succinimidyl-(CH₂)ₘ—C(═O)-PEP-N(R⁶)—(C₁-C₁₂ alkyldiyl)-;
-succinimidyl-(CH₂)ₘ—C(═O)-PEP-N(R⁶)—(C₁-C₁₂ alkyldiyl)N(R⁶)C(═O)—; and
-succinimidyl-(CH₂)ₘ—C(═O)-PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ—; m is an integer from 1 to 5, and n is an integer from 2 to 50;
Glue has the formula: 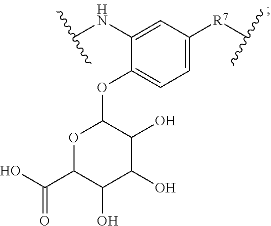
PEP has the formula: 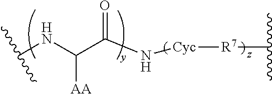
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl , NO₂, —OH, —OCH₃, and a glucuronic acid having the structure: 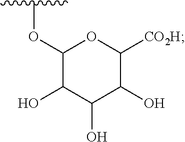 [SMILES: *OC1OC(C(=O)O)C(O)C(O)C1O]
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH(OH)CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘ—CO₂H, —O(CH₂CH₂O),H, —OCH₂F, —OCHF₂, —OCF₃, —OP(O)(OH)₂, —S(O)₂N(CH₃)₂, —SCH₃, —S(O)₂CH₃, and —S(O)₃H.

2-9. (canceled)

10. The immunoconjugate of claim 1 wherein R^{1a} and R^{1b} are independently selected from a group consisting of optionally substituted C₆-C₂₀ aryl, C₂-C₉ heterocyclyl, and C₁-C₂₀ heteroaryl.

11. The immunoconjugate of claim 1 wherein R^{1a} is optionally substituted C₆-C₂₀ aryl and R^{1b} is H.

12. The immunoconjugate of claim 1 wherein R^{1a} and R^{1b} form a five- or six-membered heterocyclyl ring.

13. The immunoconjugate of claim 1 wherein X² and X³ are each a bond, and R² and R³ are independently selected from a group consisting of C₁-C₈ alkyl, —O—(C₁-C₁₂ alkyl), —(C₁-C₁₂ alkyldiyl)-OR⁵, —(C₁-C₅ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

14. The immunoconjugate of claim 1 wherein X² is a bond, and R² is C₁-C₁₂ alkyl.

15. The immunoconjugate of claim 1 wherein X³ is 0 and R³ is —(C₁-C₁₂ alkyldiyl)-N(R⁵)—*.

16. The immunoconjugate of claim 15 wherein R³ is —CH₂CH₂NH—.

17. The immunoconjugate of claim 1 wherein L is selected from —C(═O)-PEG-C(═O)—, and —C(═O)-PEG-O—C(═O)—.

18. (canceled)

19. The immunoconjugate of claim 1 wherein n is 10 and m is 1.

20-23. (canceled)

24. The immunoconjugate of claim 1 having Formula Ia:

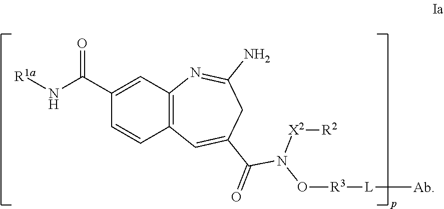

25. The immunoconjugate of claim 24 wherein R^{1a} is a group selected from optionally substituted C₆-C₂₀ aryl, C₂-C₉ heterocyclyl, and C₁-C₂₀ heteroaryl.

26. The immunoconjugate of claim 25 wherein R^{1a} is pyrimidinyl or pyridyl.

27. The immunoconjugate of claim 24 wherein X² is a bond, and R² is C₁-C₁₂ alkyl.

28. The immunoconjugate of claim 24 wherein R³ is —(C₁-C₁₂ alkyldiyl)-N(R⁵)—*.

29. The immunoconjugate of claim 1 wherein X³—R³-L is selected from the group consisting of: 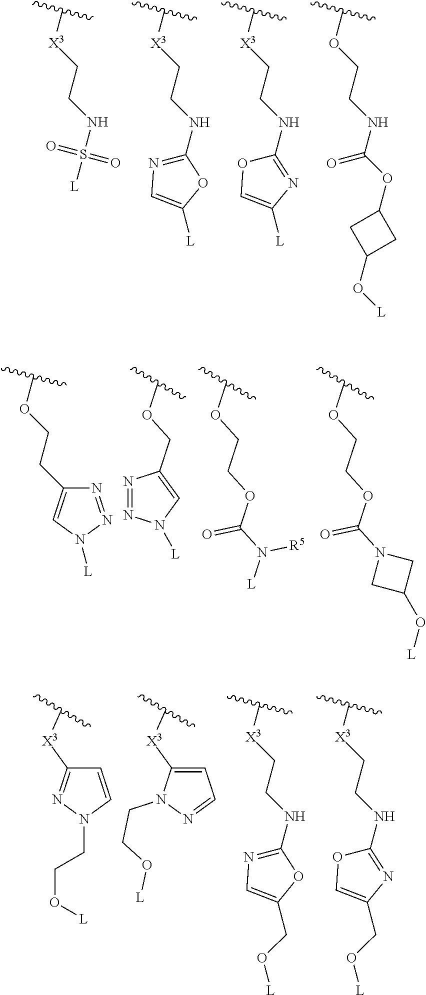
where the wavy line indicates the point of attachment to N.

30. (canceled)

31. (canceled)

32. The 2-amino-4-carboxamide-benzazepine-linker compound of claim 44 selected from the group consisting of:

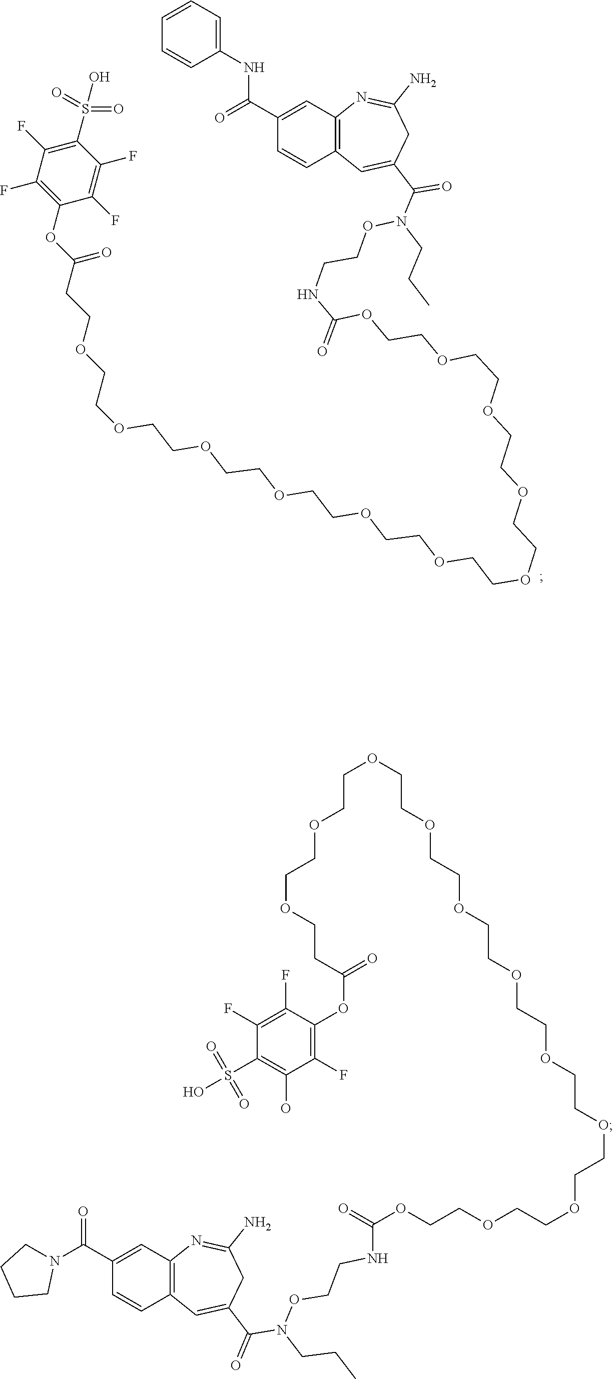 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)Nc3ccccc3)cc2N=C(N)C1 ; CCCN(OCCNC(=O)OCCOCCOCCOCCOCCOCCOCCOCCOCCOCCOCCC(=O)Oc1c(F)c(O)c(S(=O)(=O)O)c(F)c1F)C(=O)C1=Cc2ccc(C(=O)N3CCCC3)cc2N=C(N)C1]

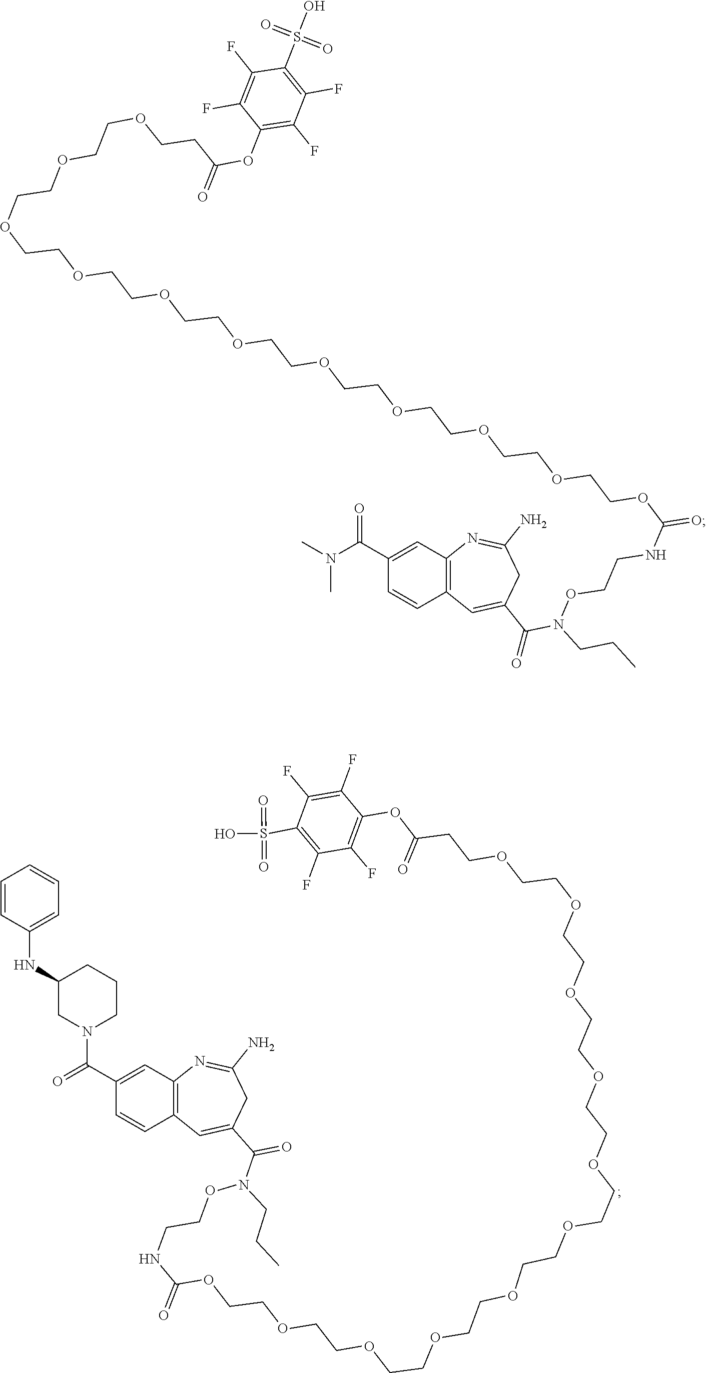 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N(C)C)cc2N=C(N)C1 ; CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCC[C@H](Nc4ccccc4)C3)cc2N=C(N)C1]

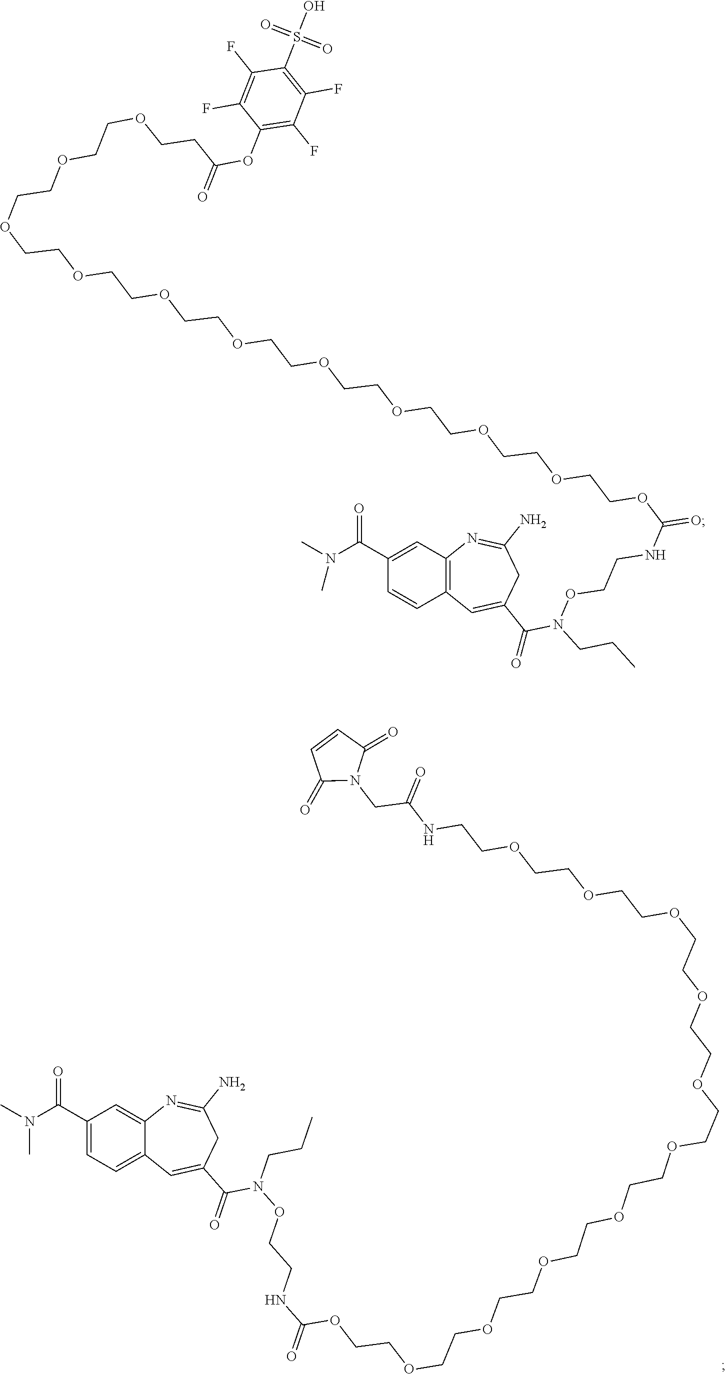 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N(C)C)cc2N=C(N)C1 ; CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1]

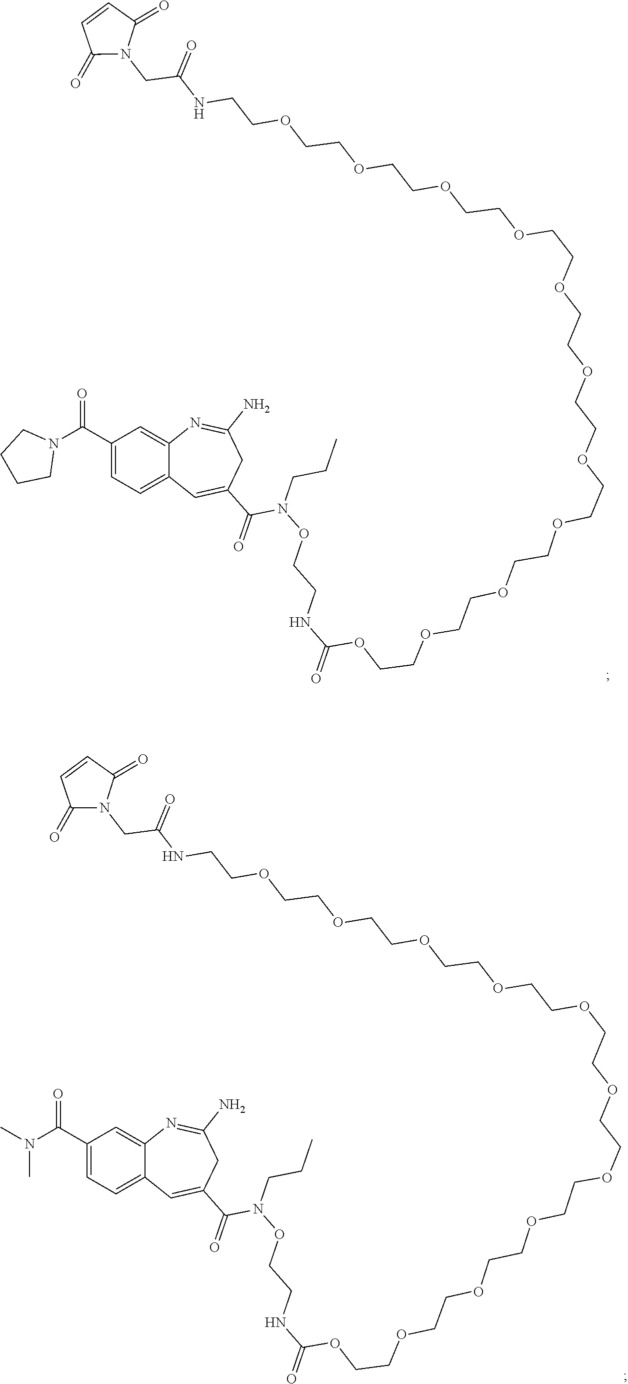 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCCC3)cc2N=C(N)C1 ; CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1]

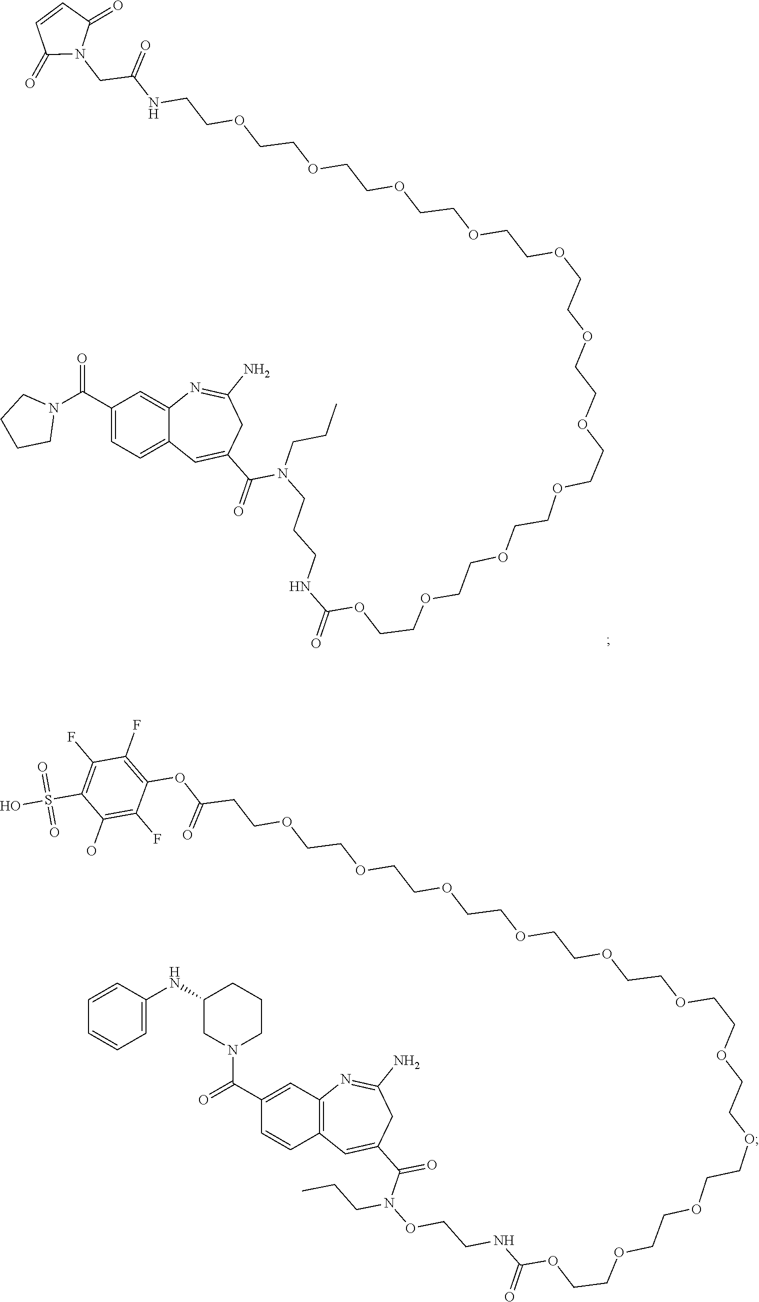 [SMILES: CCCN(CCCNC(=O)OCCOCCOCCOCCOCCOCCOCCOCCOCCOCCOCCNC(=O)CN1C(=O)C=CC1=O)C(=O)C1=Cc2ccc(C(=O)N3CCCC3)cc2N=C(N)C1 ; CCCN(OCCNC(=O)OCCOCCOCCOCCOCCOCCOCCOCCOCCOCCOCCC(=O)Oc1c(F)c(O)c(S(=O)(=O)O)c(F)c1F)C(=O)C1=Cc2ccc(C(=O)N3CCC[C@@H](Nc4ccccc4)C3)cc2N=C(N)C1]

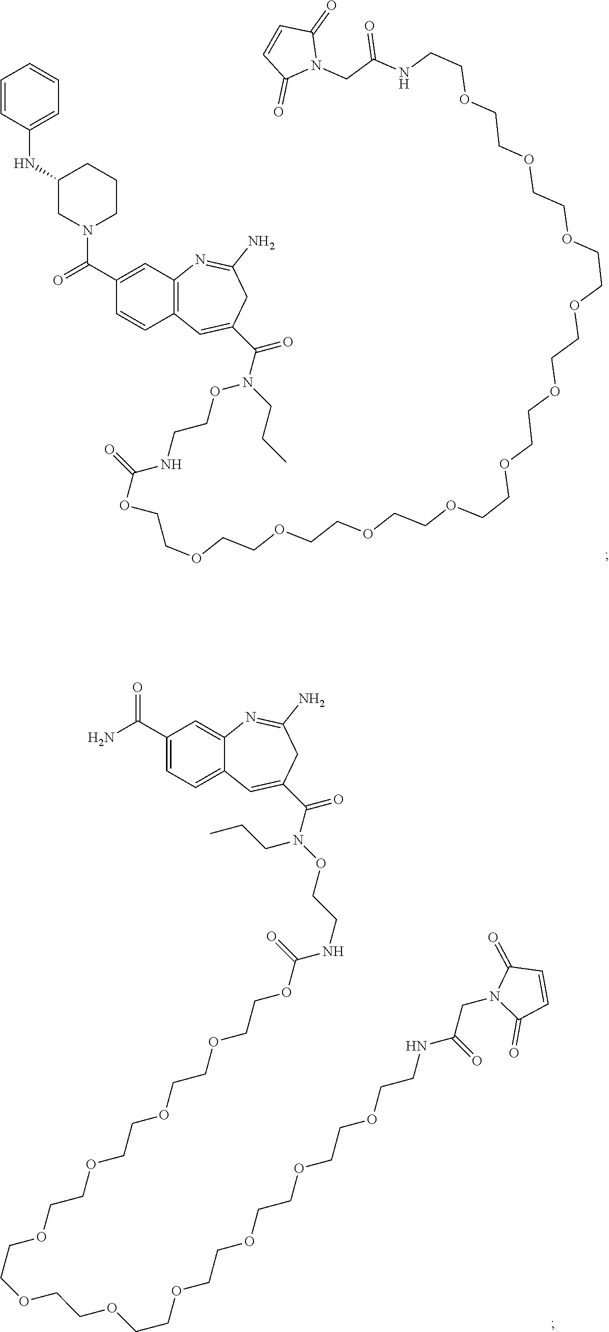 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCC[C@@H](Nc4ccccc4)C3)cc2N=C(N)C1 ; CCCN(OCCNC(=O)OCCOCCOCCOCCOCCOCCOCCOCCOCCOCCOCCNC(=O)CN1C(=O)C=CC1=O)C(=O)C1=Cc2ccc(C(N)=O)cc2N=C(N)C1]

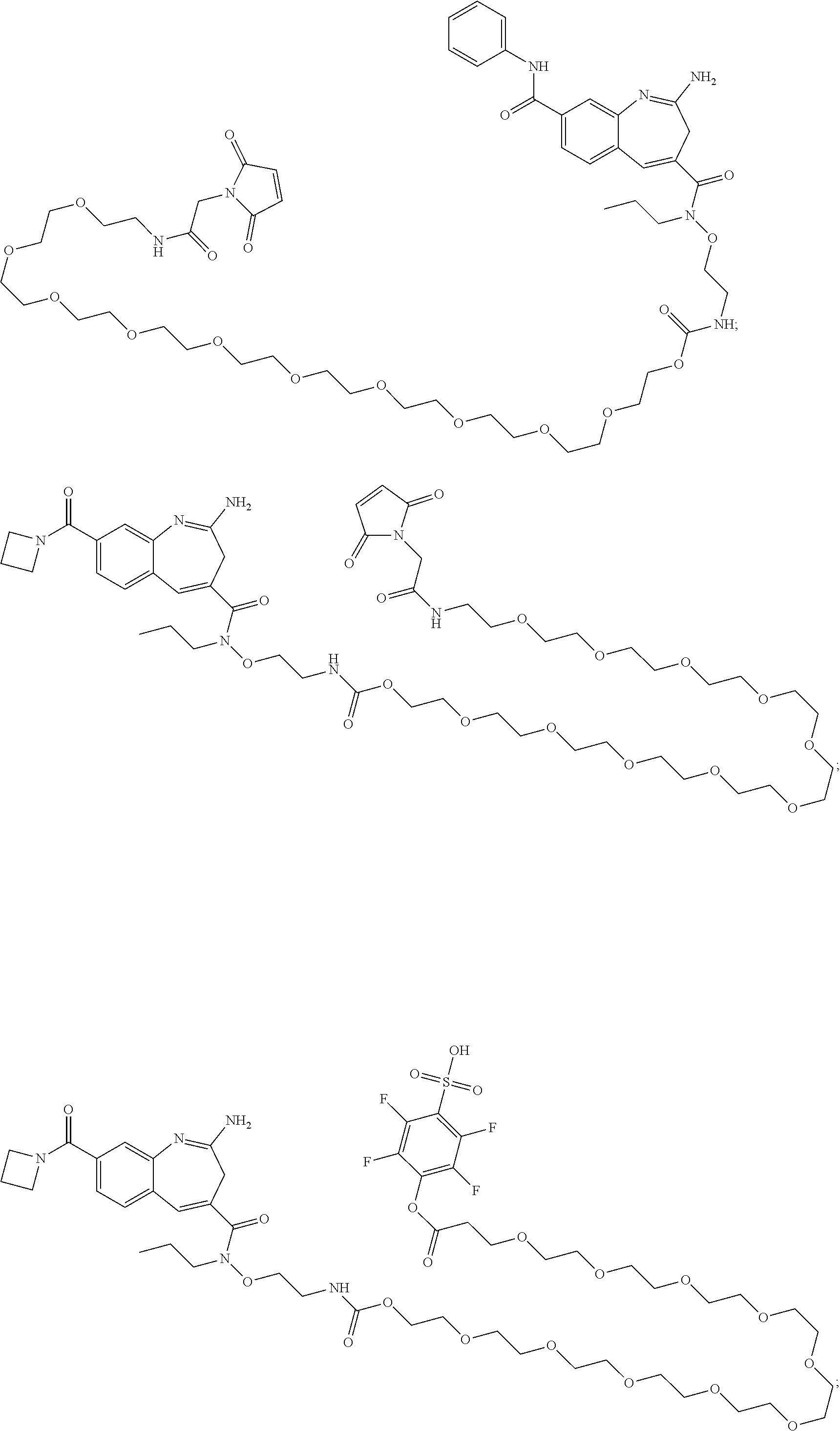 [SMILES: CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)Nc3ccccc3)cc2N=C(N)C1 ; CCCN(OCCNC(=O)OCCOCCOCCOCCOCCOCCOCCOCCOCCOCCOCCNC(=O)CN1C(=O)C=CC1=O)C(=O)C1=Cc2ccc(C(=O)N3CCC3)cc2N=C(N)C1 ; CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)N3CCC3)cc2N=C(N)C1]

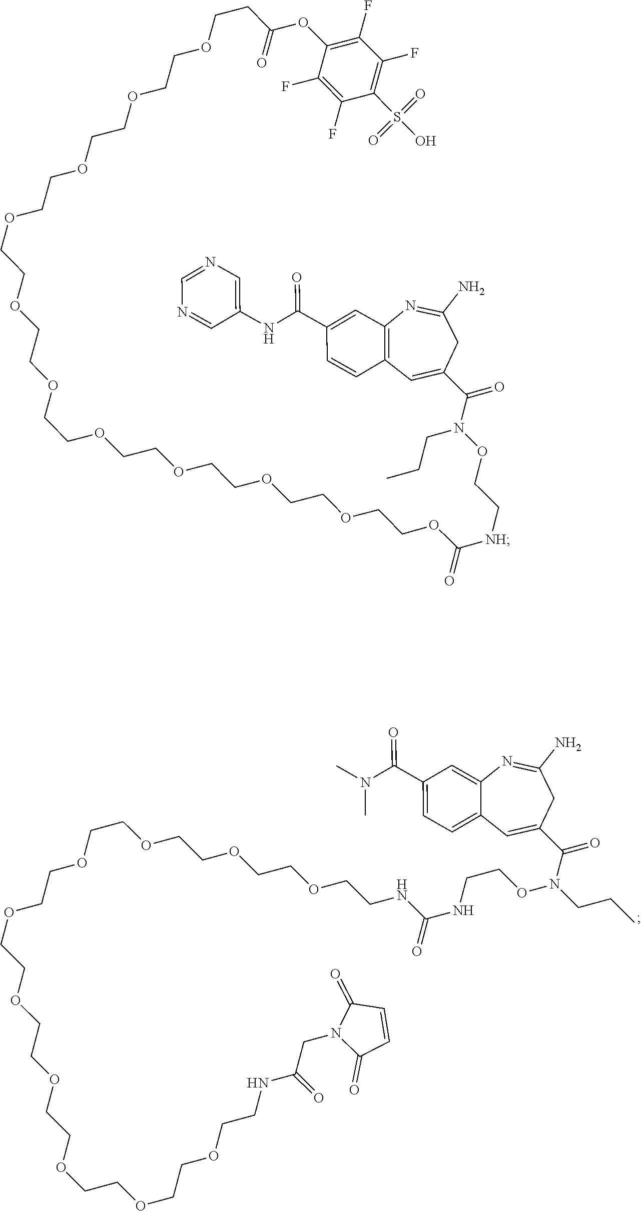 [SMILES: CCCN(OCCNC(=O)OCCOCCOCCOCCOCCOCCOCCOCCOCCOCCOCCC(=O)Oc1c(F)c(F)c(S(=O)(=O)O)c(F)c1F)C(=O)C1=Cc2ccc(C(=O)Nc3cncnc3)cc2N=C(N)C1 ; CCCN(OCCNC(=O)NCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1]

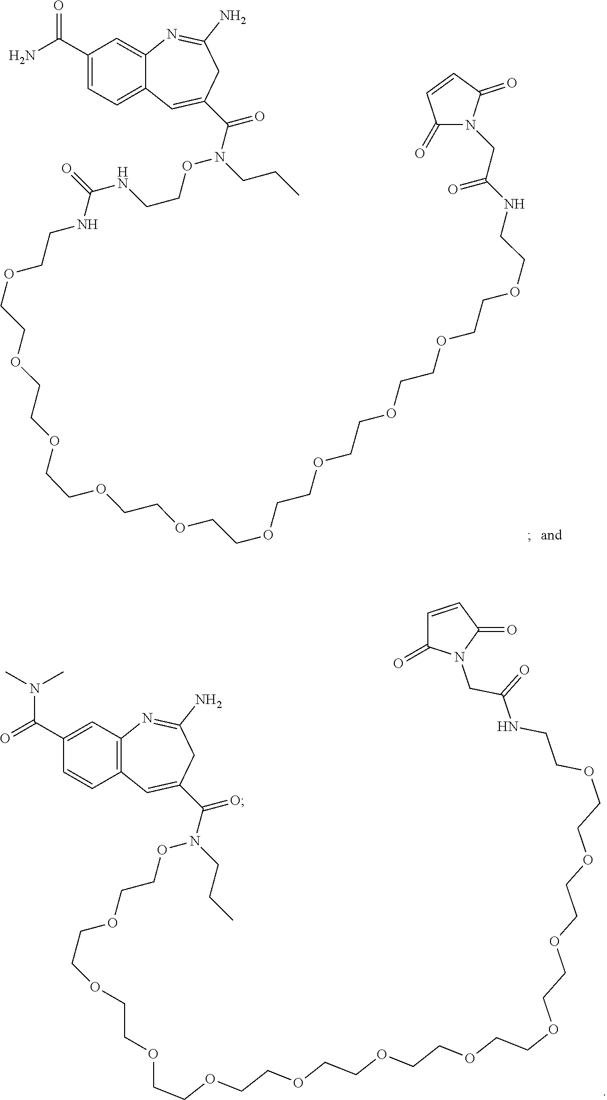 [SMILES: CCCN(OCCNC(=O)NCCOCCOCCOCCOCCOCCOCCOCCOCCOCCOCCNC(=O)CN1C(=O)C=CC1=O)C(=O)C1=Cc2ccc(C(N)=O)cc2N=C(N)C1 ; CCCN(OCCOCCOCCOCCOCCOCCOCCOCCOCCOCCOCCOCCNC(=O)CN1C(=O)C=CC1=O)C(=O)C1=Cc2ccc(C(=O)N(C)C)cc2N=C(N)C1]

33. An immunoconjugate prepared by conjugation of an antibody with a 2-amino-4-carboxamide-benzazepine-linker compound of claim 32.

34. A pharmaceutical composition comprising a therapeutically effective amount of an immunoconjugate according to claim 1, and one or more pharmaceutically acceptable diluent, vehicle, carrier or excipient.

35. A method for treating cancer comprising administering a therapeutically effective amount of an immunoconjugate according to claim 1, to a patient in need thereof, wherein the cancer is selected from bladder cancer, urinary tract cancer, urothelial carcinoma, lung cancer, non-small cell lung cancer, Merkel cell carcinoma, colon cancer, colorectal cancer, gastric cancer, and breast cancer.

36. The method of claim 35, wherein the cancer is susceptible to a pro-inflammatory response induced by TLR7 and/or TLR8 agonism.

37. The method of claim 35, wherein the cancer is selected from triple-negative breast cancer, metastatic Merkel cell carcinoma, and gastroesophageal junction adenocarcinoma.

38. (canceled)

39. (canceled)

40. A method of preparing an immunoconjugate of Formula I of claim 1 wherein the 2-amino-4-carboxamide-benzazepine-linker of claim 32 is conjugated with the antibody.

41. (canceled)

42. The immunoconjugate of claim 1 wherein the antibody binds to a target selected from the group consisting of PD-L1, HER2, TROP2, and CEA.

43. The immunoconjugate of claim 42 wherein the antibody is selected from the group consisting of trastuzumab, pertuzumab, labetuzumab, and sacituzumab.

44. The immunoconjugate of claim 1 wherein L is selected from:
-succinimidyl-(CH₂)ₘ—C(═O)N(R⁶)-PEG-;
-succinimidyl-(CH₂)ₘ—C(═O)N(R⁶)-PEG-O—;
-succinimidyl-(CH₂)ₘ—C(═O)N(R⁶)-PEG-O—C(═O)—;
-succinimidyl-(CH₂)ₘ—C(═O)N(R⁶)-PEG-C(═O)—;
-succinimidyl-(CH₂)ₘ—C(═O)N(R⁶)-PEG-N(R⁵)—;
-succinimidyl-(CH₂)ₘ—C(═O)N(R⁶)-PEG-N(R⁵)—C(═O)—;
-succinimidyl-(CH₂)ₘ—C(═O)N(R⁶)-PEG-C(═O)-PEP-; and
-succinimidyl-(CH₂)ₘ—C(═O)N(R⁶)-PEG-SS-(C₁-C₁₂ alkyldiyl)-OC(═O)—.

45. A 2-amino-4-carboxamide-benzazepine-linker compound having Formula II: 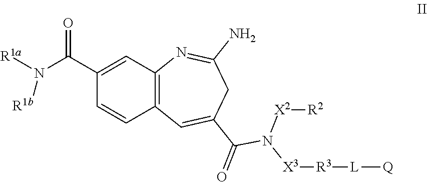
wherein
X² and X³ are independently selected from the group consisting of a bond, C(═O), C(═O)N(R⁵), O, N(R⁵), S, S(O)₂, and S(O)₂N(R⁵);
R^{1a}, R^{1b}, and R² are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl;
R³ is selected from the group consisting of:
—(C₁-C₁₂ alkyldiyl)-N(R⁵)—*;
—(C₁-C₁₂ alkyldiyl)-N(R⁵)—C(═O)*;
—(C₁-C₁₂ alkyldiyl)-N(R⁵)—C(═O)O—(C₃-C₁₂ carbocyclyldiyl)-*;
—(C₁-C₁₂ alkyldiyl)-N(R⁵)—(C₁-C₂₀ heteroaryldiyl)-*;
—(C₁-C₁₂ alkyldiyl)-N(R⁵)—(C₁-C₂₀ heteroaryldiyl)-(C₁-C₁₂ alkyldiyl)-*;
—(C₁-C₁₂ alkyldiyl)-N(R⁵)—S(O₂)—*;
—(C₁-C₁₂ alkyldiyl)-OC(═O)—(C₂-C₉ heterocyclyldiyl)-*;
—(C₁-C₁₂ alkyldiyl)-O—*;
—(C₁-C₁₂ alkyldiyl)-(C₃-C₁₂ carbocyclyldiyl)-*;
—(C₁-C₁₂ alkyldiyl)-(C₆-C₂₀ aryldiyl)-*;
—(C₁-C₁₂ alkyldiyl)-(C₆-C₂₀ aryl)-(C₁-C₁₂ alkyldiyl)-N(R⁵)—*;
—(C₁-C₁₂ alkyldiyl)-(C₆-C₂₀ aryl)-(C₁-C₁₂ alkyldiyl)-N(R⁵)—*;
—(C₁-C₁₂ alkyldiyl)-(C₂-C₉ heterocyclyldiyl)-(C₁-C₁₂ alkyldiyl)-N(R⁵)—*;
—(C₁-C₁₂ alkyldiyl)-(C₁-C₂₀ heteroaryldiyl)-N(R⁵)—*;
—(C₁-C₁₂ alkyldiyl)-(C₁-C₂₀ heteroaryldiyl)-*;
—(C₁-C₁₂ alkyldiyl)-(C₁-C₂₀ heteroaryldiyl)-(C₁-C₁₂ alkyldiyl)-*;
—(C₁-C₁₂ alkyldiyl)-(C₁-C₂₀ heteroaryldiyl)-(C₁-C₁₂ alkyldiyl)-N(R⁵)—*;
—(C₃-C₁₂ carbocyclyldiyl)-*;
—(C₃-C₁₂ carbocyclyldiyl)-(C₁-C₁₂ alkyldiyl)-N(R⁵)—*;
—(C₃-C₁₂ carbocyclyldiyl)-(C₁-C₁₂ alkyldiyl)-N(R⁵)—*;
—(C₃-C₁₂ carbocyclyldiyl)-NR⁵—C(═NR^{5a})—N(R⁵)—*;
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)—C(═NR^{5a})—N(R⁵)—*;
—(C₂-C₂₀ heterocyclyldiyl)-*;
—(C₂-C₉ heterocyclyldiyl)-(C₁-C₁₂ alkyldiyl)-N(R⁵)—*;
—(C₂-C₉ heterocyclyldiyl)-N(R⁵)—C(═NR^{5a})—N(R⁵)—*;
—(C₁-C₂₀ heteroaryldiyl)-*;
—(C₁-C₂₀ heteroaryldiyl)-(C₁-C₁₂ alkyldiyl)-N(R⁵)—*;
—(C₁-C₂₀ heteroaryldiyl)-(C₁-C₁₂ alkyldiyl)-O—*; and
—(C₁-C₂₀ heteroaryldiyl)-N(R⁵)—C(═NR^{5a})—N(R⁵)—*;
where the asterisk * indicates the attachment site of the linker L;
R⁵ is selected from the group consisting of H, C₆-C₂₀ aryl and C₁-C₁₂ alkyl, or two R⁵ groups together form a 5- or 6-membered heterocyclyl ring;
R^{5a} is selected from the group consisting of C₆-C₂₀ aryl and C₁-C₂₀ heteroaryl;
L-Q is selected from the group consisting of:
Q-C(═O)-PEG-;
Q-C(═O)-PEG-C(═O)N(R⁶)—(C₁-C₁₂ alkyldiyl)-C(═O)-Gluc-;
Q-C(═O)-PEG-O—;
Q-C(═O)-PEG-O—C(═O)—;
Q-C(═O)-PEG-C(═O)—;
Q-C(═O)-PEG-C(═O)-PEP-;
Q-C(═O)-PEG-N(R⁶)—;
Q-C(═O)-PEG-N(R⁶)—C(═O)—;
Q-C(═O)-PEG-N(R⁶)-PEG-C(═O)-PEP-;
Q-C(═O)-PEG-N⁺(R⁶)₂-PEG-C(═O)-PEP-;
Q-C(═O)-PEG-C(═O)-PEP-N(R⁶)—(C₁-C₁₂ alkyldiyl)-;
Q-C(═O)-PEG-C(═O)-PEP-N(R⁶)—(C₁-C₁₂ alkyldiyl)N(R⁶)C(═O)—(C₂-C₅ monoheterocyclyldiyl)-;
Q-C(═O)-PEG-SS-(C₁-C₁₂ alkyldiyl)-OC(═O)—;
Q-C(═O)-PEG-SS-(C₁-C₁₂ alkyldiyl)-C(═O)—;
Q-C(═O)—(C₁-C₁₂ alkyldiyl)-C(═O)-PEP-;
Q-C(═O)—(C₁-C₁₂ alkyldiyl)-C(═O)-PEP-N(R⁶)—(C₁-C₁₂ alkyldiyl)-;
Q-C(═O)—(C₁-C₁₂ alkyldiyl)-C(═O)-PEP-N(R⁶)—(C₁-C₁₂ alkyldiyl)-N(R⁵)—C(═O);
Q-C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
Q-(CH₂)ₘ—C(═O)N(R⁶)-PEG-;
Q-(CH₂)ₘ—C(═O)N(R⁶)-PEG-C(═O)N(R⁶)—(C₁-C₁₂ alkyldiyl)-C(═O)-Gluc-;
Q-(CH₂)ₘ—C(═O)N(R⁶)-PEG-O—;
Q-(CH₂)ₘ—C(═O)N(R⁶)-PEG-O—C(═O)—;
Q-(CH₂)ₘ—C(═O)N(R⁶)-PEG-C(═O)—;
Q-(CH₂)ₘ—C(═O)N(R⁶)-PEG-N(R⁵)—;
Q-(CH₂)ₘ—C(═O)N(R⁶)-PEG-N(R⁵)—C(═O)—;
Q-(CH₂)ₘ—C(═O)N(R⁶)-PEG-C(═O)-PEP-;
Q-(CH₂)ₘ—C(═O)N(R⁶)-PEG-SS-(C₁-C₁₂ alkyldiyl)-OC(═O)—;
Q-(CH₂)ₘ—C(═O)-PEP-N(R⁶)—(C₁-C₁₂ alkyldiyl)-;
Q-(CH₂)ₘ—C(═O)-PEP-N(R⁶)—(C₁-C₁₂ alkyldiyl)N(R⁶)C(═O)—; and
Q-(CH₂)ₘ—C(═O)-PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ— where m is an integer from 1 to 5, and n is an integer from 2 to 50;
Glue has the formula: 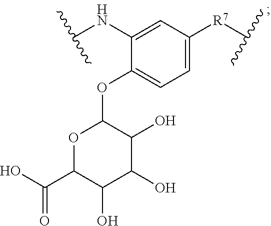
PEP has the formula: 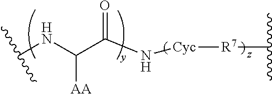
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 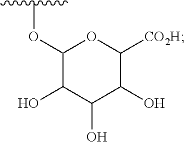 [SMILES: *OC1OC(C(=O)O)C(O)C(O)C1O]
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1;
Q is selected from the group consisting of N-hydroxysuccinimidyl, N-hydroxysulfosuccinimidyl, maleimide, and phenoxy substituted with one or more groups independently selected from F, Cl, NO₂, and SO₃⁻; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH(OH)CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘ—CO₂H, —O(CH₂CH₂O),H, —OCH₂F, —OCHF₂, —OCF₃, —OP(O)(OH)₂, —S(O)₂N(CH₃)₂, —SCH₃, —S(O)₂CH₃, and —S(O)₃H.

46. The 2-amino-4-carboxamide-benzazepine-linker compound of claim 45 wherein X² is a bond, and R² is C₁-C₁₂ alkyl.

47. The 2-amino-4-carboxamide-benzazepine-linker compound of claim 45 wherein X³ is O and R³ is —(C₁-C₁₂ alkyldiyl)-N(R⁵)—*.

48. The 2-amino-4-carboxamide-benzazepine-linker compound of claim 47 wherein R³ is —CH₂CH₂NH—.

49. The 2-amino-4-carboxamide-benzazepine-linker compound of claim 45 wherein L is selected from —C(═O)-PEG-C(═O)—, and —C(═O)-PEG-O—C(═O)—.

50. The 2-amino-4-carboxamide-benzazepine-linker compound of claim 45 wherein Q is selected from:

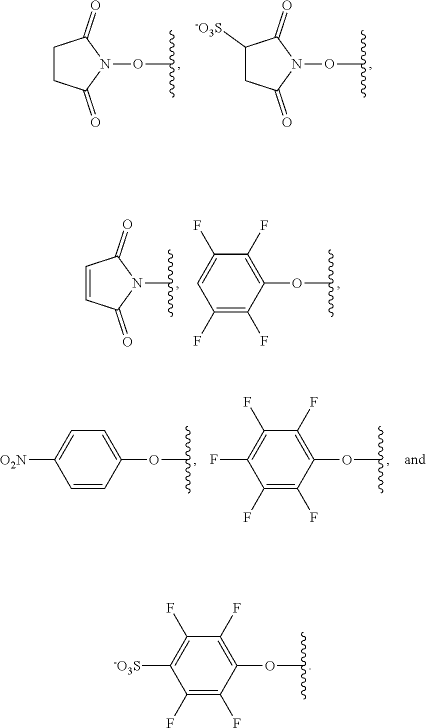

51. The 2-amino-4-carboxamide-benzazepine-linker compound of claim 50 wherein Q is 2,3,5,6-tetrafluorophenoxy or 2,3,5,6-tetrafluoro, 4-sulfonate-phenoxy.

52. The 2-amino-4-carboxamide-benzazepine-linker compound of claim 50 wherein Q is maleimide.

53. The 2-amino-4-carboxamide-benzazepine-linker compound of claim 45 having Formula IIa: [chemical structure]


SEQUENCE LISTING (175 sequences)

SEQ ID NO: 1 - protein, 106 aa, Artificial Sequence
  source: /note=“Description of Artificial Sequence Synthetic polypeptide”
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 2 - protein, 23 aa, Artificial Sequence
  source: /note=“Description of Artificial Sequence Synthetic peptide”
DIQLTQSPSSLSASVGDRVTITC

SEQ ID NO: 3 - protein, 11 aa, Artificial Sequence
  source: /note=“Description of Artificial Sequence Synthetic peptide”
KASQDVGTSVA

SEQ ID NO: 4 - protein, 15 aa, Artificial Sequence
  source: /note=“Description of Artificial Sequence Synthetic peptide”
WYQQKPGKAPKLLIY

SEQ ID NO: 5 - protein, 7 aa, Artificial Sequence
  source: /note=“Description of Artificial Sequence Synthetic peptide”
WTSTRHT

SEQ ID NO: 6 - protein, 32 aa, Artificial Sequence
  source: /note=“Description of Artificial Sequence Synthetic polypeptide”
GVPSRFSGSGSGTDFTFTISSLQPEDIATYYC

SEQ ID NO: 7 - protein, 8 aa, Artificial Sequence
  source: /note=“Description of Artificial Sequence Synthetic peptide”
QQYSLYRS

SEQ ID NO: 8 - protein, 10 aa, Artificial Sequence
  source: /note=“Description of Artificial Sequence Synthetic peptide”
FGQGTKVEIK

SEQ ID NO: 9 - protein, 119 aa, Artificial Sequence
  source: /note=“Description of Artificial Sequence Synthetic polypeptide”
EVQLVESGGGVVQPGRSLRLSCSSSGFDFTTYWMSWVRQAPGKGLEWVAEIHPDSSTINYAPSLKDRFTISRDNSKNTLFLQMDSLRPEDTGVYFCASLYFGFPWFAYWGQGTPVTVSS

SEQ ID NO: 10 - protein, 30 aa, Artificial Sequence
  source: /note=“Description of Artificial Sequence Synthetic polypeptide”
EVQLVESGGGVVQPGRSLRLSCSSSGFDFT

SEQ ID NO: 11 - protein, 5 aa, Artificial Sequence
  source: /note=“Description of Artificial Sequence Synthetic peptide”
TYWMS

SEQ ID NO: 12 - protein, 14 aa, Artificial Sequence
  source: /note=“Description of Artificial Sequence Synthetic peptide”
WVRQAPGKGLEWVA

SEQ ID NO: 13 - protein, 17 aa, Artificial Sequence
  source: /note=“Description of Artificial Sequence Synthetic peptide”
EIHPDSSTINYAPSLKD

SEQ ID NO: 14 - protein, 32 aa, Artificial Sequence
  source: /note=“Description of Artificial Sequence Synthetic polypeptide”
RFTISRDNSKNTLFLQMDSLRPEDTGVYFCAS

SEQ ID NO: 15 - protein, 10 aa, Artificial Sequence
  source: /note=“Description of Artificial Sequence Synthetic peptide”
LYFGFPWFAY

SEQ ID NO: 16 - protein, 11 aa, Artificial Sequence
  source: /note=“Description of Artificial Sequence Synthetic peptide”
WGQGTPVTVSS

SEQ ID NO: 17 - protein, 108 aa, Artificial Sequence
  source: /note=“Description of Artificial Sequence Synthetic polypeptide”
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 18 - protein, 23 aa, Artificial Sequence
  source: /note=“Description of Artificial Sequence Synthetic peptide”
DIQMTQSPSSLSASVGDRVTITC

SEQ ID NO: 19 - protein, 11 aa, Artificial Sequence
  source: /note=“Description of Artificial Sequence Synthetic peptide”
KASAAVGTYVA

SEQ ID NO: 20 - protein, 15 aa, Artificial Sequence
  source: /note=“Description of Artificial Sequence Synthetic peptide”
WYQQKPGKAPKLLIY

SEQ ID NO: 21 - protein, 7 aa, Artificial Sequence
  source: /note=“Description of Artificial Sequence Synthetic peptide”
SASYRKR

SEQ ID NO: 22 - protein, 32 aa, Artificial Sequence
  source: /note=“Description of Artificial Sequence Synthetic polypeptide”
GVPSRFSGSGSGTDFTLTISSLQPEDFATYYC

SEQ ID NO: 23 - protein, 10 aa, Artificial Sequence
  source: /note=“Description of Artificial Sequence Synthetic peptide”
HQYYTYPLFT

SEQ ID NO: 24 - protein, 10 aa, Artificial Sequence
  source: /note=“Description of Artificial Sequence Synthetic peptide”
FGQGTKLEIK

SEQ ID NO: 25 - protein, 30 aa, Artificial Sequence
  source: /note=“Description of Artificial Sequence Synthetic polypeptide”
QVQLVQSGAEVKKPGASVKVSCKASGYTFT

SEQ ID NO: 26 - protein, 5 aa, Artificial Sequence
  source: /note=“Description of Artificial Sequence Synthetic peptide”
EFGMN

SEQ ID NO: 27 - protein, 14 aa, Artificial Sequence
  source: /note=“Description of Artificial Sequence Synthetic peptide”
WVRQAPGQGLEWMG

SEQ ID NO: 28 - protein, 17 aa, Artificial Sequence
  source: /note=“Description of Artificial Sequence Synthetic peptide”
WINTKTGEATYVEEFKG

SEQ ID NO: 29 - protein, 32 aa, Artificial Sequence
  source: /note=“Description of Artificial Sequence Synthetic polypeptide”
RVTFTTDTSTSTAYMELRSLRSDDTAVYYCAR

SEQ ID NO: 30 - protein, 12 aa, Artificial Sequence
  source: /note=“Description of Artificial Sequence Synthetic peptide”
WDFAYYVEAMDY

SEQ ID NO: 31 - protein, 11 aa, Artificial Sequence
  source: /note=“Description of Artificial Sequence Synthetic peptide”
WGQGTTVTVSS

SEQ ID NO: 32 - protein, 106 aa, Artificial Sequence
  source: /note=“Description of Artificial Sequence Synthetic polypeptide”
ENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIK

SEQ ID NO: 33 - protein, 23 aa, Artificial Sequence
  source: /note=“Description of Artificial Sequence Synthetic peptide”
ENVLTQSPSSMSASVGDRVNIAC

SEQ ID NO: 34 - protein, 23 aa, Artificial Sequence
  source: /note=“Description of Artificial Sequence Synthetic peptide”
EIVLTQSPSSMSASVGDRVNIAC

SEQ ID NO: 35 - protein, 10 aa, Artificial Sequence
  source: /note=“Description of Artificial Sequence Synthetic peptide”
SASSSVSYMH

SEQ ID NO: 36 - protein, 15 aa, Artificial Sequence
  source: /note=“Description of Artificial Sequence Synthetic peptide”
WFQQKPGKSPKLWIY

SEQ ID NO: 37 - protein, 7 aa, Artificial Sequence
  source: /note=“Description of Artificial Sequence Synthetic peptide”
STSNLAS

SEQ ID NO: 38 - protein, 32 aa, Artificial Sequence
  source: /note=“Description of Artificial Sequence Synthetic polypeptide”
GVPSRFSGSGSGTDYSLTISSMQPEDAATYYC

SEQ ID NO: 39 - protein, 9 aa, Artificial Sequence
  source: /note=“Description of Artificial Sequence Synthetic peptide”
QQRSSYPLT

SEQ ID NO: 40 - protein, 10 aa, Artificial Sequence
  source: /note=“Description of Artificial Sequence Synthetic peptide”
FGGGTKLEIK

SEQ ID NO: 41 - protein, 120 aa, Artificial Sequence
  source: /note=“Description of Artificial Sequence Synthetic polypeptide”
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 42 - protein, 30 aa, Artificial Sequence
  source: /note=“Description of Artificial Sequence Synthetic polypeptide”
QVKLEQSGAEVVKPGASVKLSCKASGFNIK

SEQ ID NO: 43 - protein, 30 aa, Artificial Sequence
  source: /note=“Description of Artificial Sequence Synthetic polypeptide”
QVQLVQSGAEVVKPGASVKLSCKASGFNIK

SEQ ID NO: 44 - protein, 5 aa, Artificial Sequence
  source: /note=“Description of Artificial Sequence Synthetic peptide”
DSYMH

SEQ ID NO: 45 - protein, 14 aa, Artificial Sequence
  source: /note=“Description of Artificial Sequence Synthetic peptide”
WLRQGPGQRLEWIG

SEQ ID NO: 46 - protein, 17 aa, Artificial Sequence
  source: /note=“Description of Artificial Sequence Synthetic peptide”
WIDPENGDTEYAPKFQG

SEQ ID NO: 47 - protein, 32 aa, Artificial Sequence
  source: /note=“Description of Artificial Sequence Synthetic polypeptide”
KATFTTDTSANTAYLGLSSLRPEDTAVYYCNE

SEQ ID NO: 48 - protein, 11 aa, Artificial Sequence
  source: /note=“Description of Artificial Sequence Synthetic peptide”
GTPTGPYYFDY

SEQ ID NO: 49 - protein, 11 aa, Artificial Sequence
  source: /note=“Description of Artificial Sequence Synthetic peptide”
WGQGTLVTVSS

SEQ ID NO: 50 - protein, 106 aa, Artificial Sequence
  source: /note=“Description of Artificial Sequence Synthetic polypeptide”
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 51 - protein, 23 aa, Artificial Sequence
  source: /note=“Description of Artificial Sequence Synthetic peptide”
ENVLTQSPSSMSVSVGDRVTIAC

SEQ ID NO: 52 - protein, 23 aa, Artificial Sequence
  source: /note=“Description of Artificial Sequence Synthetic peptide”
EIVLTQSPSSMSVSVGDRVTIAC

SEQ ID NO: 53 - protein, 10 aa, Artificial Sequence
  source: /note=“Description of Artificial Sequence Synthetic peptide”
SASSSVPYMH

SEQ ID NO: 54 - protein, 15 aa, Artificial Sequence
  source: /note=“Description of Artificial Sequence Synthetic peptide”
WLQQKPGKSPKLLIY

SEQ ID NO: 55 - protein, 7 aa, Artificial Sequence
  source: /note=“Description of Artificial Sequence Synthetic peptide”
LTSNLAS

SEQ ID NO: 56 - protein, 32 aa, Artificial Sequence
  source: /note=“Description of Artificial Sequence Synthetic polypeptide”
GVPSRFSGSGSGTDYSLTISSVQPEDAATYYC

SEQ ID NO: 57 - protein, 106 aa, Artificial Sequence
  source: /note=“Description of Artificial Sequence Synthetic polypeptide”
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIK

SEQ ID NO: 58 - protein, 23 aa, Artificial Sequence
  source: /note=“Description of Artificial Sequence Synthetic peptide”
QTVLSQSPAILSASPGEKVTMTC

SEQ ID NO: 59 - protein, 10 aa, Artificial Sequence
  source: /note=“Description of Artificial Sequence Synthetic peptide”
RASSSVTYIH

SEQ ID NO: 60 - protein, 15 aa, Artificial Sequence
  source: /note=“Description of Artificial Sequence Synthetic peptide”
WYQQKPGSSPKSWIY

SEQ ID NO: 61 - protein, 7 aa, Artificial Sequence
  source: /note=“Description of Artificial Sequence Synthetic peptide”
ATSNLAS

SEQ ID NO: 62 - protein, 32 aa, Artificial Sequence
  source: /note=“Description of Artificial Sequence Synthetic polypeptide”
GVPARFSGSGSGTSYSLTISRVEAEDAATYYC

SEQ ID NO: 63 - protein, 9 aa, Artificial Sequence
  source: /note=“Description of Artificial Sequence Synthetic peptide”
QHWSSKPPT

SEQ ID NO: 64 - protein, 10 aa, Artificial Sequence
  source: /note=“Description of Artificial Sequence Synthetic peptide”
FGGGTKLEIK

SEQ ID NO: 65 - protein, 121 aa, Artificial Sequence
  source: /note=“Description of Artificial Sequence Synthetic polypeptide”
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 66 - protein, 30 aa, Artificial Sequence
  source: /note=“Description of Artificial Sequence Synthetic polypeptide”
EVKLVESGGGLVQPGGSLRLSCATSGFTFT

SEQ ID NO: 67 - protein, 5 aa, Artificial Sequence
  source: /note=“Description of Artificial Sequence Synthetic peptide”
DYYMN

SEQ ID NO: 68 - protein, 14 aa, Artificial Sequence
  source: /note=“Description of Artificial Sequence Synthetic peptide”
WVRQPPGKALEWLG

SEQ ID NO: 69 - protein, 19 aa, Artificial Sequence
  source: /note=“Description of Artificial Sequence Synthetic peptide”
FIGNKANGYTTEYSASVKG

SEQ ID NO: 70 - protein, 32 aa, Artificial Sequence
  source: /note=“Description of Artificial Sequence Synthetic polypeptide”
RFTISRDKSQSILYLQMNTLRAEDSATYYCTR

SEQ ID NO: 71 - protein, 10 aa, Artificial Sequence
  source: /note=“Description of Artificial Sequence Synthetic peptide”
DRGLRFYFDY

SEQ ID NO: 72 - protein, 11 aa, Artificial Sequence
  source: /note=“Description of Artificial Sequence Synthetic peptide”
WGQGTTLTVSS

SEQ ID NO: 73 - protein, 111 aa, Artificial Sequence
  source: /note=“Description of Artificial Sequence Synthetic polypeptide”
DIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIK

SEQ ID NO: 74 - protein, 23 aa, Artificial Sequence
  source: /note=“Description of Artificial Sequence Synthetic peptide”
DIQLTQSPSSLSASVGDRVTITC

SEQ ID NO: 75 - protein, 15 aa, Artificial Sequence
  source: /note=“Description of Artificial Sequence Synthetic peptide”
RAGESVDIFGVGFLH

SEQ ID NO: 76 - protein, 15 aa, Artificial Sequence
  source: /note=“Description of Artificial Sequence Synthetic peptide”
WYQQKPGKAPKLLIY

SEQ ID NO: 77 - protein, 7 aa, Artificial Sequence
  source: /note=“Description of Artificial Sequence Synthetic peptide”
RASNLES

SEQ ID NO: 78 - protein, 32 aa, Artificial Sequence
  source: /note=“Description of Artificial Sequence Synthetic polypeptide”
GVPSRFSGSGSRTDFTLTISSLQPEDFATYYC

SEQ ID NO: 79 - protein, 9 aa, Artificial Sequence
  source: /note=“Description of Artificial Sequence Synthetic peptide”
QQTNEDPYT

SEQ ID NO: 80 - protein, 10 aa, Artificial Sequence
  source: /note=“Description of Artificial Sequence Synthetic peptide”
FGQGTKVEIK

SEQ ID NO: 81 - protein, 121 aa, Artificial Sequence
  source: /note=“Description of Artificial Sequence Synthetic polypeptide”
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSS

SEQ ID NO: 82 - protein, 30 aa, Artificial Sequence
  source: /note=“Description of Artificial Sequence Synthetic polypeptide”
EVQLVESGGGLVQPGGSLRLSCAASGFNIK

SEQ ID NO: 83 - protein, 5 aa, Artificial Sequence
  source: /note=“Description of Artificial Sequence Synthetic peptide”
DTYMH

SEQ ID NO: 84 - protein, 14 aa, Artificial Sequence
  source: /note=“Description of Artificial Sequence Synthetic peptide”
WVRQAPGKGLEWVA

SEQ ID NO: 85 - protein, 17 aa, Artificial Sequence
  source: /note=“Description of Artificial Sequence Synthetic peptide”
RIDPANGNSKYADSVKG

SEQ ID NO: 86 - protein, 32 aa, Artificial Sequence
  source: /note=“Description of Artificial Sequence Synthetic polypeptide”
RFTISADTSKNTAYLQMNSLRAEDTAVYYCAP

SEQ ID NO: 87 - protein, 12 aa, Artificial Sequence
  source: /note=“Description of Artificial Sequence Synthetic peptide”
FGYYVSDYAMAY

SEQ ID NO: 88 - protein, 11 aa, Artificial Sequence
  source: /note=“Description of Artificial Sequence Synthetic peptide”
WGQGTLVTVSS

SEQ ID NO: 89 - protein, 107 aa, Artificial Sequence [sequence omitted]

SEQ ID NO: 90 - protein, 23 aa, Artificial Sequence
  source: /note=“Description of Artificial Sequence Synthetic peptide”
DIQMTQSPASLSASVGDRVTITC

SEQ ID NO: 91 - protein, 11 aa, Artificial Sequence
  source: /note=“Description of Artificial Sequence Synthetic peptide”
RASENIFSYLA

SEQ ID NO: 92 - protein, 15 aa, Artificial Sequence
  source: /note=“Description of Artificial Sequence Synthetic peptide”
WYQQKPGKSPKLLVY

SEQ ID NO: 93 - protein, 7 aa, Artificial Sequence
  source: /note=“Description of Artificial Sequence Synthetic peptide”
NTRTLAE

SEQ ID NO: 94 - protein, 32 aa, Artificial Sequence
  source: /note=“Description of Artificial Sequence Synthetic polypeptide”
GVPSRFSGSGSGTDFSLTISSLQPEDFATYYC

SEQ ID NO: 95 - protein, 9 aa, Artificial Sequence
  source: /note=“Description of Artificial Sequence Synthetic peptide”
QHHYGTPFT

SEQ ID NO: 96 - protein, 10 aa, Artificial Sequence
  source: /note=“Description of Artificial Sequence Synthetic peptide”
FGSGTKLEIK

SEQ ID NO: 97 - protein, 120 aa, Artificial Sequence [sequence omitted]

SEQ ID NO: 98 - protein, 30 aa, Artificial Sequence
  source: /note=“Description of Artificial Sequence Synthetic polypeptide”
EVQLQESGPGLVKPGGSLSLSCAASGFVFS

SEQ ID NO: 99 - protein, 5 aa, Artificial Sequence
  source: /note=“Description of Artificial Sequence Synthetic peptide”
SYDMS

SEQ ID NO: 100 - protein, 14 aa, Artificial Sequence
  source: /note=“Description of Artificial Sequence Synthetic peptide”
WVRQTPERGLEWVA

SEQ ID NO: 101 - protein, 17 aa, Artificial Sequence
  source: /note=“Description of Artificial Sequence Synthetic peptide”
YISSGGGITYAPSTVKG

SEQ ID NO: 102 - protein, 32 aa, Artificial Sequence
  source: /note=“Description of Artificial Sequence Synthetic polypeptide”
RFTVSRDNAKNTLYLQMNSLTSEDTAVYYCAA

SEQ ID NO: 103 - protein, 11 aa, Artificial Sequence
  source: /note=“Description of Artificial Sequence Synthetic peptide”
HYFGSSGPFAY

SEQ ID NO: 104 - protein, 11 aa, Artificial Sequence
  source: /note=“Description of Artificial Sequence Synthetic peptide”
WGQGTLVTVSS

SEQ ID NO: 105 - protein, 116 aa, Artificial Sequence
  source: /note=“Description of Artificial Sequence Synthetic polypeptide”
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 106 - protein, 22 aa, Artificial Sequence
  source: /note=“Description of Artificial Sequence Synthetic peptide”
QAVLTQPASLSASPGASASLTC

SEQ ID NO: 107 - protein, 14 aa, Artificial Sequence
  source: /note=“Description of Artificial Sequence Synthetic peptide”
TLRRGINVGAYSIY

SEQ ID NO: 108 - protein, 15 aa, Artificial Sequence
  source: /note=“Description of Artificial Sequence Synthetic peptide”
WYQQKPGSPPQYLLR

SEQ ID NO: 109 - protein, 11 aa, Artificial Sequence
  source: /note=“Description of Artificial Sequence Synthetic peptide”
YKSDSDKQQGS

SEQ ID NO: 110 - protein, 34 aa, Artificial Sequence
  source: /note=“Description of Artificial Sequence Synthetic polypeptide”
GVSSRFSASKDASANAGILLISGLQSEDEADYYC

SEQ ID NO: 111 - protein, 10 aa, Artificial Sequence
  source: /note=“Description of Artificial Sequence Synthetic peptide”
MIWHSGASAV

SEQ ID NO: 112 - protein, 10 aa, Artificial Sequence
  source: /note=“Description of Artificial Sequence Synthetic peptide”
FGGGTKLTVL

SEQ ID NO: 113 - protein, 121 aa, Artificial Sequence
  source: /note=“Description of Artificial Sequence Synthetic polypeptide”
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 114 - protein, 30 aa, Artificial Sequence
  source: /note=“Description of Artificial Sequence Synthetic polypeptide”
EVQLVESGGGLVQPGRSLRLSCAASGFTVS

SEQ ID NO: 115 - protein, 5 aa, Artificial Sequence
  source: /note=“Description of Artificial Sequence Synthetic peptide”
SYWMH

SEQ ID NO: 116 - protein, 14 aa, Artificial Sequence
  source: /note=“Description of Artificial Sequence Synthetic peptide”
WVRQAPGKGLEWVG

SEQ ID NO: 117 - protein, 19 aa, Artificial Sequence
  source: /note=“Description of Artificial Sequence Synthetic peptide”
FIRNKANGGTTEYAASVKG

SEQ ID NO: 118 - protein, 19 aa, Artificial Sequence
  source: /note=“Description of Artificial Sequence Synthetic peptide”
FIRNKANSGTTEYAASVKG

SEQ ID NO: 119 - protein, 32 aa, Artificial Sequence
  source: /note=“Description of Artificial Sequence Synthetic polypeptide”
RFTISRDDSKNTLYLQMNSLRAEDTAVYYCAR

SEQ ID NO: 120 - protein, 10 aa, Artificial Sequence
  source: /note=“Description of Artificial Sequence Synthetic peptide”
DRGLRFYFDY

SEQ ID NO: 121 - protein, 11 aa, Artificial Sequence
  source: /note=“Description of Artificial Sequence Synthetic peptide”
WGQGTTVTVSS

SEQ ID NO: 122 - protein, 121 aa, Artificial Sequence
  source: /note=“Description of Artificial Sequence Synthetic polypeptide”
EVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSS

SEQ ID NO: 123 - protein, 30 aa, Artificial Sequence; identical to SEQ ID NO: 114
  source: /note=“Description of Artificial Sequence Synthetic polypeptide”

SEQ ID NO: 124 - protein, 5 aa, Artificial Sequence
  source: /note=“Description of Artificial Sequence Synthetic peptide”
SYWMH

SEQ ID NO: 125 - protein, 14 aa, Artificial Sequence
  source: /note=“Description of Artificial Sequence Synthetic peptide”
WVRQAPGKGLEWVG

SEQ ID NO: 126 - protein, 19 aa, Artificial Sequence
  source: /note=“Description of Artificial Sequence Synthetic peptide”
FILNKANGGTTEYAASVKG

SEQ ID NO: 127 - protein, 32 aa, Artificial Sequence; identical to SEQ ID NO: 119
  source: /note=“Description of Artificial Sequence Synthetic polypeptide”

SEQ ID NO: 128 - protein, 10 aa, Artificial Sequence
  source: /note=“Description of Artificial Sequence Synthetic peptide”
DRGLRFYFDY

SEQ ID NO: 129 - protein, 11 aa, Artificial Sequence
  source: /note=“Description of Artificial Sequence Synthetic peptide”
WGQGTTVTVSS

SEQ ID NO: 130 - protein, 121 aa, Artificial Sequence
  source: /note=“Description of Artificial Sequence Synthetic polypeptide”
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 131 - protein, 11 aa, Artificial Sequence
  source: /note=“Description of Artificial Sequence Synthetic peptide”
KASQDVSIAVA

SEQ ID NO: 132 - protein, 7 aa, Artificial Sequence
  source: /note=“Description of Artificial Sequence Synthetic peptide”
SASYRYT

SEQ ID NO: 133 - protein, 9 aa, Artificial Sequence
  source: /note=“Description of Artificial Sequence Synthetic peptide”
QQHYITPLT

SEQ ID NO: 134 - protein, 5 aa, Artificial Sequence
  source: /note=“Description of Artificial Sequence Synthetic peptide”
NYGMN

SEQ ID NO: 135 - protein, 17 aa, Artificial Sequence
  source: /note=“Description of Artificial Sequence Synthetic peptide”
WINTYTGEPTYTDDFKG

SEQ ID NO: 136 - protein, 12 aa, Artificial Sequence
  source: /note=“Description of Artificial Sequence Synthetic peptide”
GGFGSSYWYFDV

SEQ ID NO: 137 - protein, 17 aa, Artificial Sequence
  source: /note=“Description of Artificial Sequence Synthetic peptide”
WINTKTGEPTYAEEFKG

SEQ ID NO: 138 - protein, 12 aa, Artificial Sequence
  source: /note=“Description of Artificial Sequence Synthetic peptide”
GGYGSSYWYFDV

SEQ ID NO: 139 - protein, 17 aa, Artificial Sequence
  source: /note=“Description of Artificial Sequence Synthetic peptide”
KSSQSLLNSGNQQNYLA

SEQ ID NO: 140 - protein, 7 aa, Artificial Sequence
  source: /note=“Description of Artificial Sequence Synthetic peptide”
GASTRES

SEQ ID NO: 141 - protein, 9 aa, Artificial Sequence
  source: /note=“Description of Artificial Sequence Synthetic peptide”
QSDHIYPYT

SEQ ID NO: 142 - protein, 5 aa, Artificial Sequence
  source: /note=“Description of Artificial Sequence Synthetic peptide”
IYWLG

SEQ ID NO: 143 - protein, 17 aa, Artificial Sequence
  source: /note=“Description of Artificial Sequence Synthetic peptide”
NIFPGSAYINYNEKFKG

SEQ ID NO: 144 - protein, 7 aa, Artificial Sequence
  source: /note=“Description of Artificial Sequence Synthetic peptide”
EGSNSGY

SEQ ID NO: 145 - protein, 11 aa, Artificial Sequence
  source: /note=“Description of Artificial Sequence Synthetic peptide”
KASQDVSTAVA

SEQ ID NO: 146 - protein, 5 aa, Artificial Sequence
  source: /note=“Description of Artificial Sequence Synthetic peptide”
TAGMQ

SEQ ID NO: 147 - protein, 17 aa, Artificial Sequence
  source: /note=“Description of Artificial Sequence Synthetic peptide”
WINTHSGVPKYAEDFKG

SEQ ID NO: 148 - protein, 12 aa, Artificial Sequence
  source: /note=“Description of Artificial Sequence Synthetic peptide”
SGFGSSYWYFDV

SEQ ID NO: 149 - protein, 15 aa, Artificial Sequence
  source: /note=“Description of Artificial Sequence Synthetic peptide”
  VARIANT (10)..(10): /replace=“L” or “N”
  SITE (1)..(15): /note=“Variant residues given in the sequence have no preference with respect to those in the annotations for variant positions”
RASKSVSTSGYSYMH

SEQ ID NO: 150 - protein, 7 aa, Artificial Sequence
  source: /note=“Description of Artificial Sequence Synthetic peptide”
LASNLES

SEQ ID NO: 151 - protein, 9 aa, Artificial Sequence
  source: /note=“Description of Artificial Sequence Synthetic peptide”
QHSRELPYT

SEQ ID NO: 152 - protein, 5 aa, Artificial Sequence
  source: /note=“Description of Artificial Sequence Synthetic peptide”
SYGVH

SEQ ID NO: 153 - protein, 7 aa, Artificial Sequence
  source: /note=“Description of Artificial Sequence Synthetic peptide”
GGSISSY

SEQ ID NO: 154 - protein, 10 aa, Artificial Sequence
  source: /note=“Description of Artificial Sequence Synthetic peptide”
GGSISSYGVH

SEQ ID NO: 155 - protein, 16 aa, Artificial Sequence
  source: /note=“Description of Artificial Sequence Synthetic peptide”
  VARIANT (5)..(5): /replace=“S”
  VARIANT (7)..(7): /replace=“V”
  VARIANT (16)..(16): /replace=“G”
  SITE (1)..(16): /note=“Variant residues given in the sequence have no preference with respect to those in the annotations for variant positions”
VIWTGGSTDYNSALMS

SEQ ID NO: 156 - protein, 5 aa, Artificial Sequence
  source: /note=“Description of Artificial Sequence Synthetic peptide”
  VARIANT (3)..(3): /replace=“S”
  VARIANT (5)..(5): /replace=“V”
  SITE (1)..(5): /note=“Variant residues given in the sequence have no preference with respect to those in the annotations for variant positions”
WTGGS

SEQ ID NO: 157 - protein, 11 aa, Artificial Sequence
  source: /note=“Description of Artificial Sequence Synthetic peptide”
DGDYDRYTMDY

SEQ ID NO: 158 - protein, 15 aa, Artificial Sequence
  source: /note=“Description of Artificial Sequence Synthetic peptide”
RASKSVSTSGYSYMH

SEQ ID NO: 159 - protein, 16 aa, Artificial Sequence
  source: /note=“Description of Artificial Sequence Synthetic peptide”
VIWTSGVTDYNSALMG

SEQ ID NO: 160 - protein, 5 aa, Artificial Sequence
  source: /note=“Description of Artificial Sequence Synthetic peptide”
WTSGV

SEQ ID NO: 161 - protein, 11 aa, Artificial Sequence
  source: /note=“Description of Artificial Sequence Synthetic peptide”
KADYECHKVYA

SEQ ID NO: 162 - protein, 11 aa, Artificial Sequence
  source: /note=“Description of Artificial Sequence Synthetic peptide”
YEKHKCYACEV

SEQ ID NO: 163 - protein, 11 aa, Artificial Sequence
  source: /note=“Description of Artificial Sequence Synthetic peptide”
QLKSGCASVVC

SEQ ID NO: 164 - protein, 451 aa, Artificial Sequence
  source: /note=“Description of Artificial Sequence Synthetic polypeptide”
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 214 aa, Artificial Sequence
  source: /note=“Description of Artificial Sequence Synthetic polypeptide”
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYECHKVYACEVTHQGLSSPVTKSFNRGEC

SEQ ID NO: 166 - protein, 214 aa, Artificial Sequence
  source: /note=“Description of Artificial Sequence Synthetic polypeptide”
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKCYACEVTHQGLSSPVTKSFNRGEC

SEQ ID NO: 167 - protein, 214 aa, Artificial Sequence
  source: /note=“Description of Artificial Sequence Synthetic polypeptide”
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGCASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 11 aa, Artificial Sequence
  source: /note=“Description of Artificial Sequence Synthetic peptide”
TVSSACTKGPS

SEQ ID NO: 169 - protein, 214 aa, Artificial Sequence
  source: /note=“Description of Artificial Sequence Synthetic polypeptide”
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 448 aa, Artificial Sequence
  source: /note=“Description of Artificial Sequence Synthetic polypeptide”
QVQLVQSGAEVKKPGASVKVSCKASGDTFTNHYMHWVRQAPGQGLEWMGWINPNSGHTGYAQKFQGRVTMTRDTSTSTVYMELSSLRSEDTAVYYCAREAVAGPMDVWGQGTTVTVSSACTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 9 aa, Artificial Sequence
  source: /note=“Description of Artificial Sequence Synthetic peptide”
GGGGSSSSG

SEQ ID NO: 172 - protein, 106 aa, Artificial Sequence
  source: /note=“Description of Artificial Sequence Synthetic polypeptide”
EIVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIK

SEQ ID NO: 173 - protein, 120 aa, Artificial Sequence
  source: /note=“Description of Artificial Sequence Synthetic polypeptide”
QVQLVQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 174 - protein, 106 aa, Artificial Sequence
  source: /note=“Description of Artificial Sequence Synthetic polypeptide”
EIVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 175 - protein, 121 aa, Artificial Sequence
  source: /note=“Description of Artificial Sequence Synthetic polypeptide”
EVQLVESGGGLVQPGRSLRLSCAASGFTVSSYWMHWVRQAPGKGLEWVGFIRNKANSGTTEYAASVKGRFTISRDDSKNTLYLQMNSLRAEDTAVYYCARDRGLRFYFDYWGQGTTVTVSS